(19) United States
(12) Patent Application Publication
(10) Pub. No.: US 2022/0227867 A1
(43) Pub. Date: Jul. 21, 2022

(54) ICOS TARGETED HETERODIMERIC FUSION PROTEINS CONTAINING IL-15/IL-15RA Fc-FUSION PROTEINS AND ICOS ANTIGEN BINDING DOMAINS

(71) Applicant: Xencor, Inc., Monrovia, CA (US)

(72) Inventors: Matthew J. Bernett, Monrovia, CA (US); John R. Desjarlais, Pasadena, CA (US); Gregory Moore, Azusa, CA (US); Suzanne Schubbert, Long Beach, CA (US)

(21) Appl. No.: 17/561,613

(22) Filed: Dec. 23, 2021

Related U.S. Application Data
(60) Provisional application No. 63/130,541, filed on Dec. 24, 2020.

(51) Int. Cl. C07K 16/28; C07K 14/54; C07K 14/715; C12N 15/63

(52) U.S. Cl. CPC C07K 16/2818; C07K 14/5443; C07K 14/7155; C07K 2319/30; C07K 2317/66; C07K 2317/56; C07K 2317/53; C12N 15/63

(57) ABSTRACT

The present invention is directed to novel targeted heterodimeric Fc fusion proteins comprising an IL-15/IL-15Rα Fc-fusion protein and a ICOS antibody fragment-Fc fusion protein.

### PRIORITY CLAIM

This application claims the benefit of U.S. Provisional Patent Application No. 63/130,541, filed Dec. 24, 2020, which is hereby incorporated by reference in its entirety.

### SEQUENCE LISTING INCORPORATION PARAGRAPH

The instant application contains a Sequence Listing which has been submitted electronically in ASCII format and is hereby incorporated by reference in its entirety. Said ASCII copy, created on Dec. 15, 2021, is named 067461-5238-WO_SL.txt and is 1,302,954 bytes in size.

### BACKGROUND

Two very promising approaches in cancer immunotherapy include cytokine-based treatments and agonism of costimulatory receptors such as ICOS.

Cytokines such as IL-2 and IL-15 function in aiding the proliferation and differentiation of B cells, T cells, and NK cells. Both cytokines exert their cell signaling function through binding to a trimeric complex consisting of two shared receptors, the common gamma chain (γc; CD132) and IL-2 receptor beta-chain (IL-2Rβ; CD122), as well as an alpha chain receptor unique to each cytokine: IL-2 receptor alpha (IL-2Rα; CD25) or IL-15 receptor alpha (IL-15Rα; CD215). Both cytokines are considered as potentially valuable therapeutics in oncology, and IL-2 has been approved for use in patients with metastatic renal-cell carcinoma and malignant melanoma. Currently, there are no approved uses of recombinant IL-15, although several clinical trials are ongoing. However, as potential drugs, both cytokines suffer from a very fast clearance, with half-lives measured in minutes. IL-2 immunotherapy has been associated with systemic toxicity when administered in high doses to overcome fast clearance. Such systemic toxicity has also been reported with IL-15 immunotherapy in recent clinical trials (Guo et al. 2015).

Therefore, there remains an unmet need in oncology treatment for therapeutic strategies with cytokines which do not require high doses and are targeted to tumors to avoid systemic toxicity.

### BRIEF SUMMARY

In some aspects, provided herein is a fusion protein comprising: a) an antigen binding domain that binds human ICOS; and b) an IL-15/IL-15Rα complex.

In some aspect, provided herein is a heterodimeric protein comprising: a) a first monomer comprising, from N- to C-terminal: i) an IL-15Rα sushi domain; ii) a first domain linker; iii) a variant IL-15 domain; iv) a second domain linker; v) a first variant Fc domain comprising CH2-CH3; b) a second monomer comprising a heavy chain comprising VH1-CH1-hinge-CH2-CH3, wherein said CH2-CH3 is a second variant Fc domain; and c) a light chain comprising VL-CL; wherein said VH1 and VL form an antigen binding domain that binds human ICOS.

In other aspects, provided herein is a heterodimeric protein comprising: a) a first monomer comprising, from N- to C-terminal: i) an IL-15Rα sushi domain; ii) a first domain linker; iii) a variant IL-15 domain; iv) a second domain linker; v) a first variant Fc domain comprising CH2-CH3; and b) a second monomer comprising, from N- to C-terminal: i) a scFv domain; ii) a third domain linker; iii) a second variant Fc domain comprising CH2-CH3; wherein said scFv domain comprises a first variable heavy domain, an scFv linker and a first variable light domain, wherein said scFv domain binds human ICOS.

In some aspects, provided herein is a heterodimeric protein comprising: a) a first monomer comprising, from N- to C-terminal: i) a variant IL-15 domain; ii) a first domain linker; iii) an IL-15Rα sushi domain; iv) a second domain linker; v) a first variant Fc domain comprising CH2-CH3; and b) a second monomer comprising, from N- to C-terminal: i) a scFv domain; ii) a third domain linker; iii) a second variant Fc domain comprising CH2-CH3; wherein said scFv domain comprises a first variable heavy domain, an scFv linker and a first variable light domain, wherein said scFv domain binds human ICOS.

In some aspects, provided herein is a heterodimeric protein comprising: a) a first monomer comprising, from N- to C-terminal: i) a variant IL-15 domain; ii) a first domain linker; iii) a first variant Fc domain comprising CH2-CH3; and b) a second monomer comprising, from N- to C-terminal: i) a scFv domain; ii) a second domain linker; iii) a second variant Fc domain comprising CH2-CH3; wherein said scFv domain comprises a first variable heavy domain, an scFv linker and a first variable light domain, wherein said scFv domain binds human ICOS.

In certain aspects, provided herein is a heterodimeric protein comprising: a) a first monomer comprising, from N- to C-terminal: i) an IL-15Rα sushi domain; ii) a first domain linker; iii) a first variant Fc domain comprising CH2-CH3; b) a second monomer comprising, from N- to C-terminal: i) a scFv domain; ii) a second domain linker; iii) a second variant Fc domain comprising CH2-CH3; wherein said scFv domain comprises a first variable heavy domain, an scFv linker and a first variable light domain; and c) a third monomer comprising a variant IL-15 domain; wherein said scFv domain binds human ICOS.

In some aspects, provided herein is a heterodimeric protein comprising: a) a first monomer comprising, from N- to C-terminal: i) a variant IL15 domain; ii) a first domain linker; iii) a first variant Fc domain comprising CH2-CH3; b) a second monomer comprising, from N- to C-terminal: i) a scFv domain; ii) a second domain linker; iii) a second variant Fc domain comprising CH2-CH3; wherein said scFv domain comprises a first variable heavy domain, an scFv linker and a first variable light domain; and c) a third monomer comprising an IL15Rα sushi domain; wherein said scFv domain binds human ICOS.

In certain aspects, provided herein is a heterodimeric protein comprising: a) a first monomer comprising, from N- to C-terminal: i) a variant IL-15Rα sushi domain with a cysteine residue; ii) a first domain linker; iii) a first variant Fc domain comprising CH2-CH3; b) a second monomer comprising, from N- to C-terminal: i) a scFv domain; ii) a second domain linker; iii) a second variant Fc domain comprising CH2-CH3; wherein said scFv domain comprises a first variable heavy domain, an scFv linker and a first variable light domain; and c) a third monomer comprising a variant IL-15 domain comprising a cysteine residue; wherein said variant IL-15Rα sushi domain and said variant IL-15 domain form a disulfide bond and said scFv domain binds human ICOS.

In other aspects, provided herein is a heterodimeric protein comprising: a) a first monomer comprising, from N- to C-terminal: i) a variant IL-15 domain with a cysteine residue; ii) a first domain linker; iii) a first variant Fc domain comprising CH2-CH3; b) a second monomer comprising, from N- to C-terminal: i) a scFv domain; ii) a second domain linker; iii) a second variant Fc domain comprising CH2-CH3; wherein said scFv domain comprises a first variable heavy domain, an scFv linker and a first variable light domain; and c) a third monomer comprising a variant IL-15Rα sushi domain comprising a cysteine residue; wherein said variant IL-15Rα sushi domain and said variant IL-15 domain form a disulfide bond and said scFv domain binds human ICOS.

In some aspects, provided herein is a heterodimeric protein comprising: a) a first monomer comprising, from N- to C-terminal: i) a variant IL15 domain; ii) a first domain linker; iii) an IL-15Rα sushi domain; iv) a second domain linker; v) a first variant Fc domain comprising CH2-CH3; b) a second monomer comprising a heavy chain comprising VH1-CH1-hinge-CH2-CH3, wherein said CH2-CH3 is a second variant Fc domain; and c) a light chain comprising VL-CL; wherein said VH1 and VL form an antigen binding domain that binds human ICOS.

In some aspects, provided herein is a heterodimeric protein comprising: a) a first monomer comprising, from N- to C-terminal: i) a variant IL15 domain; ii) a domain linker; iii) a first variant Fc domain comprising CH2-CH3; b) a second monomer comprising a heavy chain comprising VH1-CH1-hinge-CH2-CH3, wherein said CH2-CH3 is a second variant Fc domain; and c) a light chain comprising VL-CL; wherein said VH1 and VL form an antigen binding domain that binds human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising, from N- to C-terminal: i) an IL-15Rα sushi domain; ii) a domain linker; iii) a first variant Fc domain comprising CH2-CH3; c) a third monomer comprising a variant IL-15 domain; and d) a fourth monomer comprising a light chain comprising VL-CL; wherein said VH1 and VL form an antigen binding domain that binds human ICOS.

In certain aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising, from N- to C-terminal: i) a variant IL-15 domain; ii) a domain linker; iii) a first variant Fc domain comprising CH2-CH3; c) a third monomer comprising an IL-15Rα sushi domain; and d) a fourth monomer comprising a light chain comprising VL-CL; wherein said VH1 and VL form an antigen binding domain that binds human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising, from N- to C-terminal: i) a variant IL-15Rα sushi domain with a cysteine residue; ii) a domain linker; iii) a first variant Fc domain comprising CH2-CH3; c) a third monomer comprising a variant IL-15 domain comprising a cysteine residue; and d) a fourth monomer comprising a light chain comprising VL-CL; wherein said variant IL-15Rα sushi domain and said variant IL-15 domain form a disulfide bond and said VH1 and VL form an antigen binding domain that binds human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising, from N- to C-terminal: i) a variant IL-15 domain with a cysteine residue; ii) a domain linker; iii) a first variant Fc domain comprising CH2-CH3; c) a third monomer comprising a variant IL-15Rα sushi domain comprising a cysteine residue; and d) a fourth monomer comprising a light chain comprising VL-CL; wherein said variant IL-15Rα sushi domain and said variant IL-15 domain form a disulfide bond and said VH1 and VL form an antigen binding domain that binds human ICOS.

In certain aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-IL-15Rα sushi domain-domain linker-IL-15 variant, wherein said CH2-CH3 is a second variant Fc domain; and c) a third monomer comprising a light chain comprising VL-CL; wherein said VH1 and VL form antigen binding domains that bind human ICOS.

In other aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-IL-15 variant-domain linker-IL15Rα sushi domain, wherein said CH2-CH3 is a second variant Fc domain; and c) a third monomer comprising a light chain comprising VL-CL; wherein said VH1 and VL form antigen binding domains that bind human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-IL-15 variant, wherein said CH2-CH3 is a second variant Fc domain; c) a third monomer comprising a light chain comprising VL-CL; wherein said VH1 and VL form antigen binding domains that bind human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-IL-15Rα sushi domain, wherein said CH2-CH3 is a second variant Fc domain; c) a third monomer comprising a variant IL-15 domain; and d) a fourth monomer comprising a light chain comprising VL-CL; wherein said VH1 and VL form antigen binding domains that bind human ICOS.

In other aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-IL-15 variant, wherein said CH2-CH3 is a second variant Fc domain; c) a third monomer comprising an IL-15Rα sushi domain; and d) a fourth monomer comprising a light chain comprising VL-CL; wherein said VH1 and VL form antigen binding domains that bind human ICOS.

In some aspects, provided herein is a heterodimeric protein comprising: a) a first monomer comprising a heavy chain comprising VH1-CH1-hinge-CH2-CH3-domain linker-IL-15Rα sushi domain, wherein said CH2-CH3 is a first variant Fc domain; b) a second monomer comprising VH1-CH1-hinge-CH2-CH3-domain linker-IL-15 variant, wherein said CH2-CH3 is a second variant Fc domain; and d) a third monomer comprising a light chain comprising VL-CL; wherein said VH1 and VL form antigen binding domains that bind human ICOS.

In certain aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-variant IL-15Rα sushi domain, wherein said variant IL-15Rα sushi domain comprises a cysteine residue wherein said CH2-CH3 is a second variant Fc domain; c) a third monomer comprising a variant IL-15 domain comprising a cysteine residue; and d) a fourth monomer comprising a light chain comprising VL-CL; wherein said variant IL-15Rα sushi domain and said variant IL-15 domain form a disulfide bond and said VH1 and VL form antigen binding domains that bind human ICOS.

In some aspects, provided herein is a heterodimeric protein comprising: a) a first monomer comprising a heavy chain comprising VH1-CH1-hinge-CH2-CH3, wherein said CH2-CH3 is a first variant Fc domain; b) a second monomer comprising VH1-CH1-hinge-CH2-CH3-domain linker-variant IL-15 domain, wherein said variant IL-15 domain comprises a cysteine residue wherein said CH2-CH3 is a second variant Fc domain; c) a third monomer comprising a variant IL-15Rα sushi domain comprising a cysteine residue; and d) a fourth monomer comprising a light chain comprising VL-CL; wherein said variant IL-15Rα sushi domain and said variant IL-15 domain form a disulfide bond and said VH1 and VL form antigen binding domains that bind human ICOS.

In some aspects, provided herein is a heterodimeric protein comprising: a) a first monomer comprising a heavy chain comprising VH1-CH1-hinge-CH2-CH3-domain linker-variant IL-15Rα sushi domain, wherein said variant IL-15Rα sushi domain comprises a cysteine residue wherein said CH2-CH3 is a first variant Fc domain; b) a second monomer comprising VH1-CH1-hinge-CH2-CH3-domain linker-IL-15 variant, wherein said variant IL-15 domain comprises a cysteine residue wherein said CH2-CH3 is a second variant Fc domain; and d) a third monomer comprising a light chain comprising VL-CL; wherein said variant IL-15Rα sushi domain and said variant IL-15 domain form a disulfide bond and said VH1 and VL form antigen binding domains that bind human ICOS.

In exemplary aspects, provided herein is a heterodimeric protein comprising: a) a first monomer comprising, from N- to C-terminal, a VH-CH1-domain linker-variant IL-15 domain-domain linker-CH2-CH3, wherein said CH2-CH3 is a first variant Fc domain; b) a second monomer comprising, from N- to C-terminal, a VH-CH1-domain linker-IL-15Rα sushi domain-domain linker-CH2-CH3, wherein said CH2-CH3 is a second variant Fc domain; and c) a third monomer comprising a light chain comprising VL-CL; wherein said VH and said VL form antigen binding domains that bind human ICOS.

In some aspects, provided herein is a heterodimeric protein comprising: a) a first monomer comprising, from N- to C-terminal, a VH-CH1-domain linker-variant IL-15 domain-domain linker-CH2-CH3, wherein said variant IL-15 domain comprises a cysteine residue wherein said CH2-CH3 is a first variant Fc domain; b) a second monomer comprising, from N- to C-terminal, a VH-CH1-domain linker-variant IL-15Rα sushi domain-domain linker-CH2-CH3, wherein said variant IL-15Rα sushi domain comprises a cysteine residue wherein said CH2-CH3 is a second variant Fc domain; and c) a third monomer comprising a light chain comprising VL-CL; wherein said variant IL-15Rα sushi domain and said variant IL-15 domain form a disulfide bond and said VH and said VL form antigen binding domains that bind human ICOS.

In some aspects, provided herein is a heterodimeric protein comprising: a) a first monomer comprising, from N- to C-terminal, a VH-CH1-domain linker-variant IL-15 domain-domain linker-CH2-CH3, wherein said CH2-CH3 is a first variant Fc domain; b) a second monomer comprising, from N- to C-terminal, a VH-CH1-domain linker-variant IL-15 domain-domain linker-CH2-CH3, wherein said CH2-CH3 is a second variant Fc domain; and c) a third monomer comprising a light chain comprising VL-CL; wherein said VH and said VL form antigen binding domains that bind human ICOS.

In certain aspects, provided herein is a heterodimeric protein comprising: a) a first monomer comprising from N- to C-terminal, VH-CH1-domain linker-IL-15Rα sushi domain-domain linker-variant IL-15 domain-domain linker-CH2-CH3, wherein said CH2-CH3 is a first variant Fc domain; b) a second monomer comprising a heavy chain comprising VH-CH1-hinge-CH2-CH3, wherein said CH2-CH3 is a second variant Fc domain; and c) a third monomer comprising a light chain comprising VL-CL; wherein said VH and said VL form antigen binding domains that bind human ICOS.

In other aspects, provided herein is a heterodimeric protein comprising: a) a first monomer comprising from N- to C-terminal, VH-CH1-domain linker-variant IL-15 domain-domain linker-IL-15Rα sushi domain-domain linker-CH2-CH3, wherein said CH2-CH3 is a first variant Fc domain; b) a second monomer comprising a heavy chain comprising VH-CH1-hinge-CH2-CH3, wherein said CH2-CH3 is a second variant Fc domain; and c) a third monomer comprising a light chain comprising VL-CL; wherein said VH and said VL form antigen binding domains that bind human ICOS.

In some aspects, provided herein is a heterodimeric protein comprising: a) a first monomer comprising from N- to C-terminal, VH-CH1-domain linker-variant IL-15 domain-domain linker-CH2-CH3, wherein said CH2-CH3 is a first variant Fc domain; b) a second monomer comprising a heavy chain comprising VH-CH1-hinge-CH2-CH3, wherein said CH2-CH3 is a second variant Fc domain; and c) a third monomer comprising a light chain comprising VL-CL; wherein said VH and said VL form antigen binding domains that bind human ICOS.

In some embodiments of the heterodimeric proteins described herein, the VH and VL are selected from the pairs selected from the group consisting of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In exemplary embodiments of the heterodimeric proteins described herein, the first and the second Fc domains have a set of amino acid substitutions selected from the group consisting of S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W; T366S/L368A/Y407V/Y349C:T366W/S354C; and T366S/L368A/Y407V/5354C:T366W/Y349C, according to EU numbering. In some embodiments, the first and the second Fc domains have S364K/E357Q:L368D/K370S.

In exemplary embodiments of the heterodimeric proteins described herein, the variant Fc domains each comprise M428L/N434S.

In some embodiments, of the heterodimeric proteins described herein, the variant Fc domains each comprise E233P/L234V/L235A/G236del/S267K.

In exemplary embodiments of the heterodimeric proteins described herein, the variant IL-15 domain comprises an amino acid substitution(s) selected from the group consisting of N1D, N4D, D8N, D30N, V49R, D61N, E64Q, N65D, N72D, Q108E, N4D/N65D, D30N/N65D, D30N/E64Q/N65D, N1G/D30N/E46G/V49R/E64Q, N1A/D30N/E46G/V49R, and D22N/Y26F/E46Q/E53Q/E89Q/E93Q.

In exemplary embodiments, the heterodimeric protein is selected from the group consisting of XENP29975, XENP29978, XENP30810, XENP30811, XENP30812 and XENP30813.

In other aspects, provided herein is a method of treating a patient in need thereof comprising administering to said patient any of the heterodimeric proteins or pharmaceutical compositions described herein.

In some aspects, provided herein is a fusion protein that includes an antigen binding domain that binds human ICOS; and an IL-15.

In some aspects, the present invention provides a method of treating a patient in need thereof comprising administering to the patient any one of the heterodimeric fusion proteins described herein or a pharmaceutical composition described herein. In some embodiments, the method of treating further comprising administering an antibody selected from the group consisting of an anti-PD-1 antibody, an anti-PD-L1 antibody, an anti-CTLA-4 antibody, an anti-TIM-3 antibody, an anti-LAG-3 antibody, or an anti-TIGIT antibody.

Nucleic acids, expression vectors and host cells are all provided as well, in addition to methods of making these proteins and treating patients with them.

### BRIEF DESCRIPTION OF THE DRAWINGS

FIG. 1 depicts the structure of IL-15 in complex with its receptors IL-15Rα (CD215), IL-15Rβ (CD122), and the common gamma chain (CD132).

FIGS. 2A-2B depict the sequences for IL-15 and its receptors.

FIG. 3 depicts the sequences for ICOS for both human and cynomolgus monkey to facilitate the development of antigen binding domains that bind to both for ease of clinical development.

FIGS. 4A-4F depict useful pairs of Fc heterodimerization variant sets (including skew and pI variants). There are variants for which there are no corresponding “monomer 2” variants; these are pI variants which can be used alone on either monomer.

FIG. 5 depict a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein.)

FIG. 6 depict useful ablation variants that ablate FcγR binding (sometimes referred to as “knock outs” or “KO” variants). Generally, ablation variants are found on both monomers, although in some cases they may be on only one monomer.

FIGS. 7A-7E shows particularly useful embodiments of “non-cytokine”/“non-Fv” components of the IL-15/Rα-Fc fusion proteins described herein.

FIGS. 8A-8F show particularly useful embodiments of “non-cytokine”/“non-Fv” components of the ICOS-targeted IL-15/Rα-Fc fusion proteins of the invention.

FIG. 9 depicts a number of exemplary variable length linkers (e.g., domain linkers) for use in IL-15/Rα-Fc fusion proteins. In some embodiments, these linkers find use linking the C-terminus of IL-15 and/or IL-15Rα(sushi) to the N-terminus of the Fc region. In some embodiments, these linkers find use fusing IL-15 (including the IL-15 variant) to the IL-15Rα(sushi).

FIG. 10 depicts a number of charged scFv linkers that find use in increasing or decreasing the pI of heterodimeric antibodies that utilize one or more scFv as a component. The (+H) positive linker finds particular use herein. A single prior art scFv linker with single charge is referenced as “Whitlow”, from Whitlow et al., Protein Engineering 6(8):989-995 (1993). It should be noted that this linker was used for reducing aggregation and enhancing proteolytic stability in scFvs.

FIGS. 11A-11D depict the sequences of several useful IL-15/Rα-Fc format backbones based on human IgG1, without the cytokine sequences (e.g., the IL-15 and/or IL-15Rα(sushi)). It is important to note that these backbones can also find use in certain embodiments of ICOS-targeted IL-15/Rα-Fc fusion proteins. Backbone 1 is based on human IgG1 (356E/358M allotype), and includes C220S on both chains, the S364K/E357Q:L368D/K370S skew variants, the Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes C220S on both chains, the S364K:L368D/K370S skew variants, the Q295E/N384D/Q418E/N421D pI variants on the chain with L368D/K370S skew variants and the E233P/L234V/L235A/G236del/S267K ablation variants on both chains. Backbone 3 is based on human IgG1 (356E/358M allotype), and includes C220S on both chains, the S364K:L368E/K370S skew variants, the Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes C220S on both chains, the D401K:K360E/Q362E/T411E skew variants, the Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes C220S on both chains, the S364K/E357Q:L368D/K370S skew variants, the Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes C220S on both chains, the S364K/E357Q:L368D/K370S skew variants, Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Alternative formats for backbones 6 and 7 can exclude the ablation variants E233P/L234V/L235A/G236del/S267K in both chains. Backbone 8 is based on human IgG4, and includes the S364K/E357Q:L368D/K370S skew variants, the Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q:L368D/K370S skew variants, the Q295E/N384D/Q418E/N421D pI variants on the chain with L368D/K370S skew variants. Backbone 10 is based on human IgG2, and includes the S364K/E357Q:L368D/K370S skew variants, the Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes C220S on both identical chains, the E233P/L234V/L235A/G236del/S267K ablation variants on both identical chains. Backbone 13 is based on human IgG1 (356E/358M allotype), and includes C220S on both chains, the S364K/E357Q:L368D/K370S skew variants, the P217R/P229R/N276K pI variants on the chain with S364K/E357Q skew variants and the E233P/L234V/L235A/G236del/S267K ablation variants on both chains.

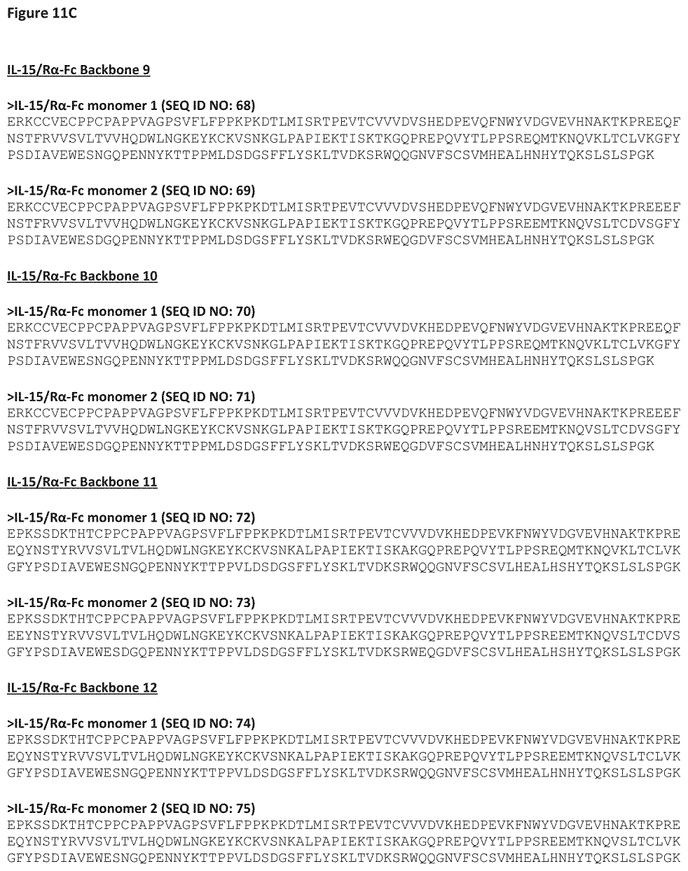

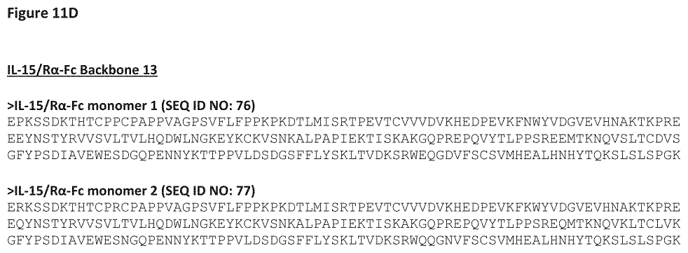

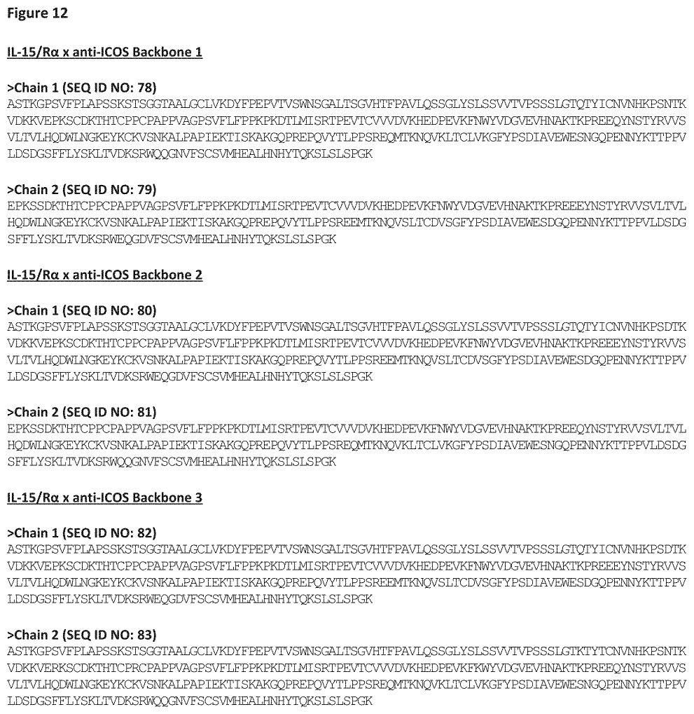

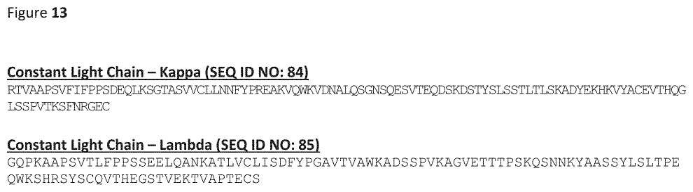

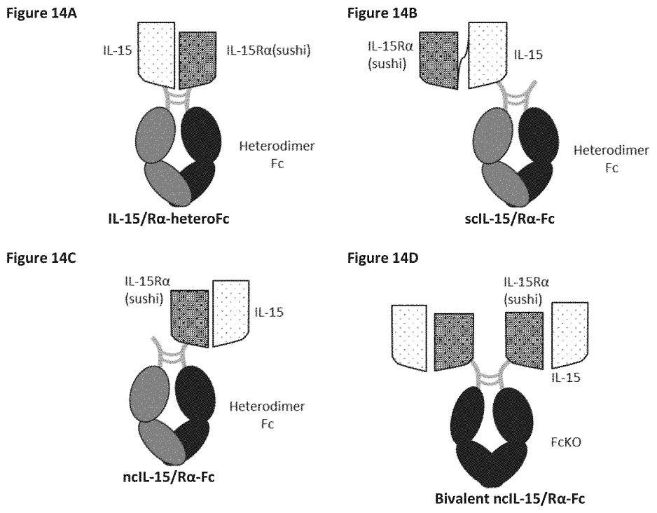

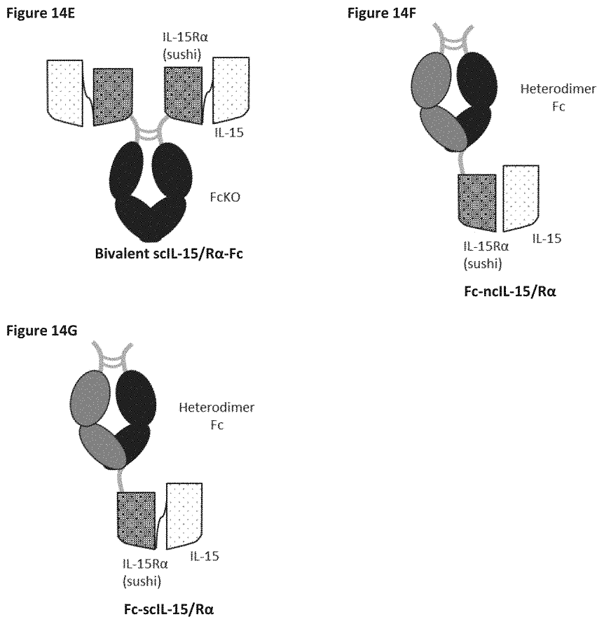

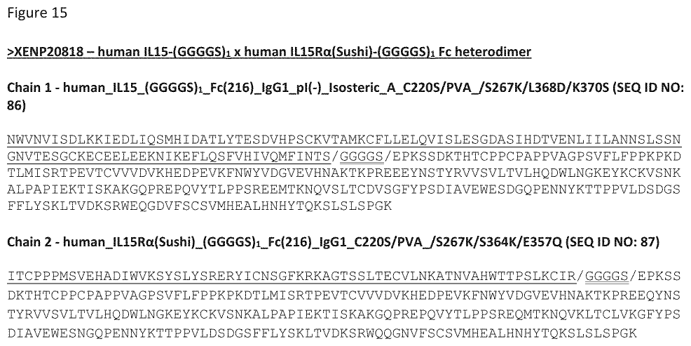

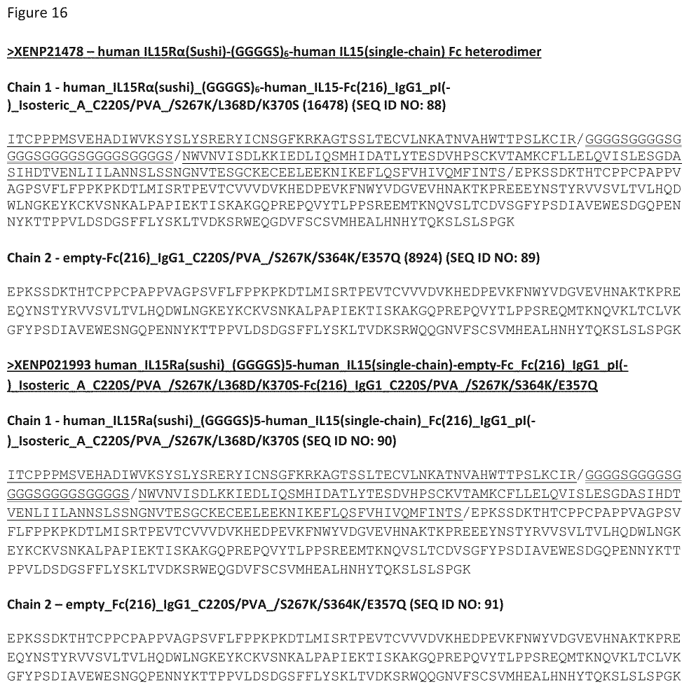

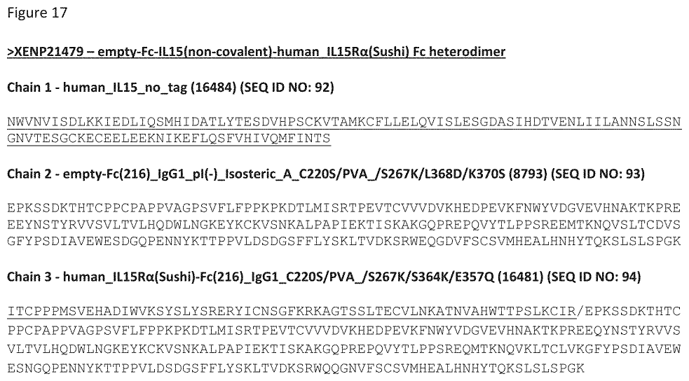

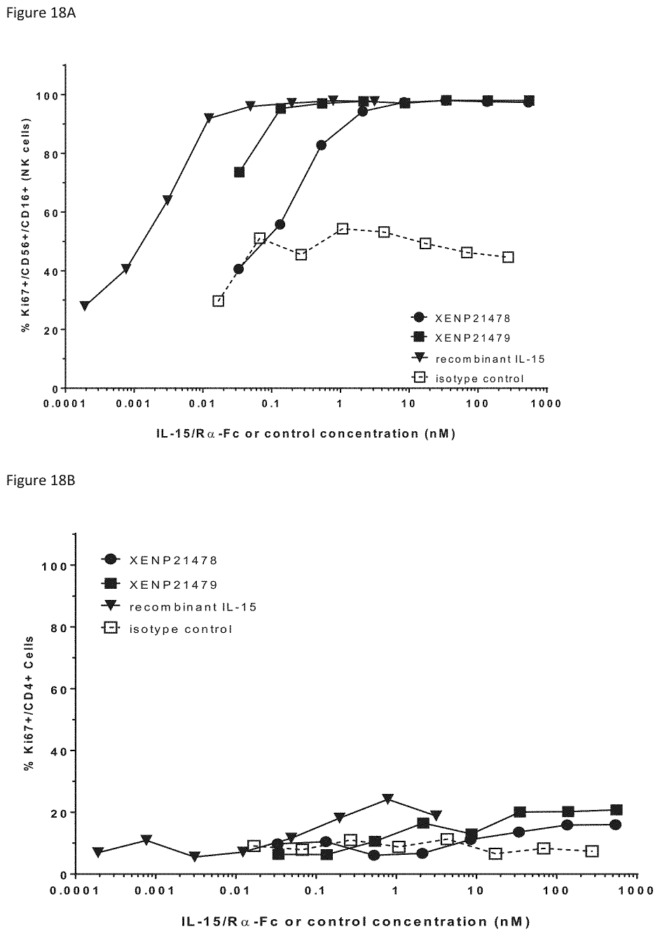

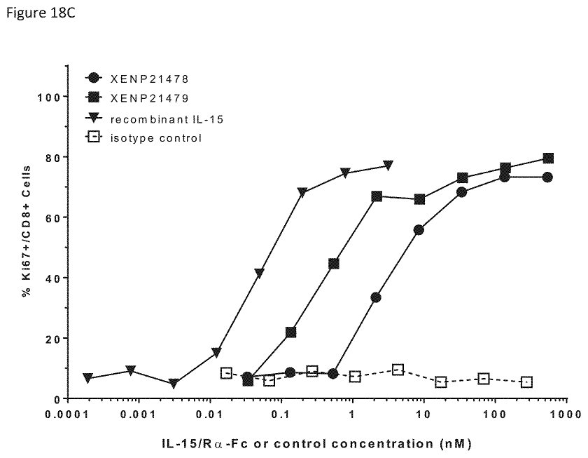

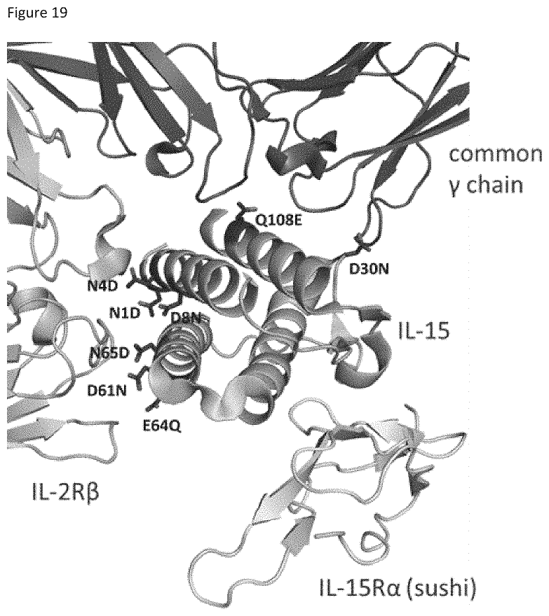

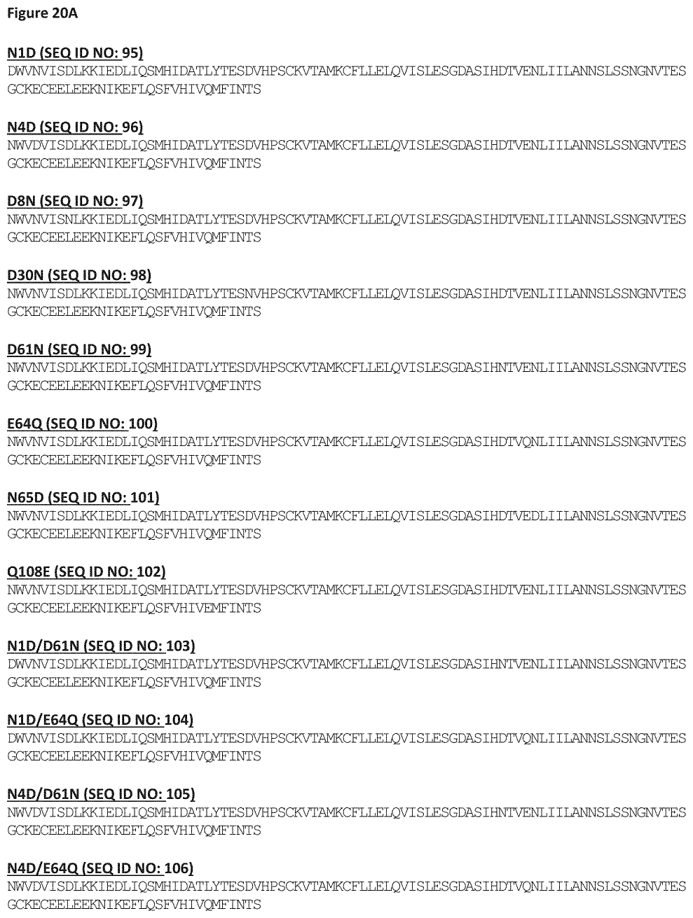

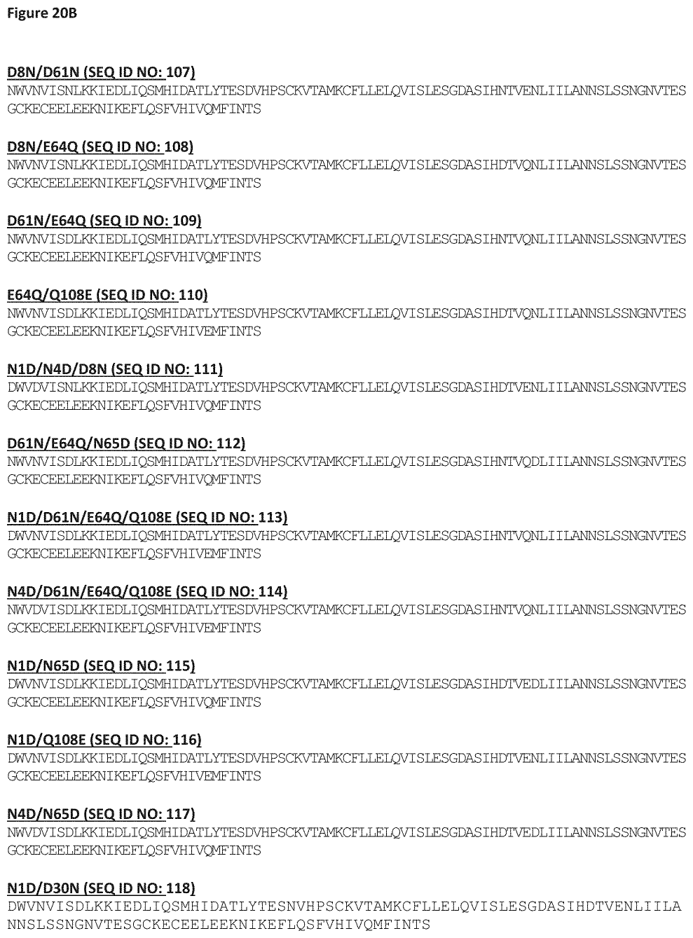

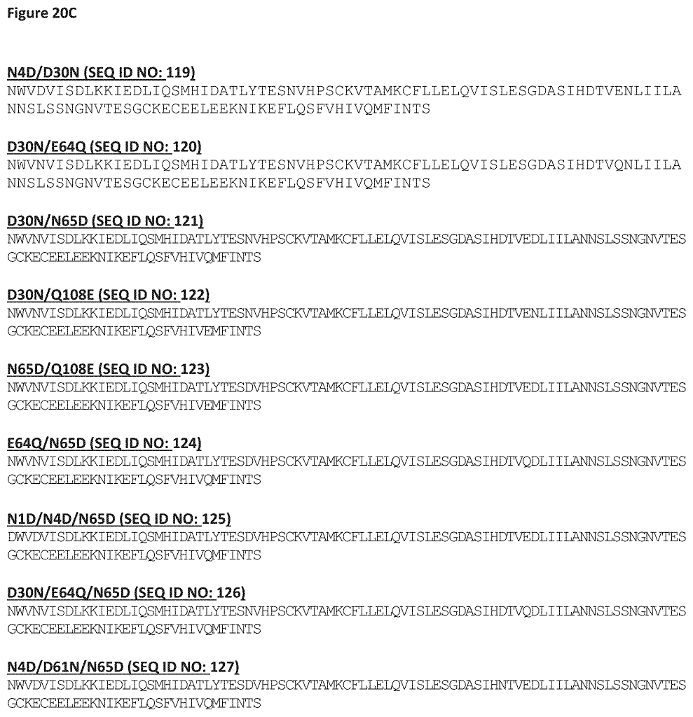

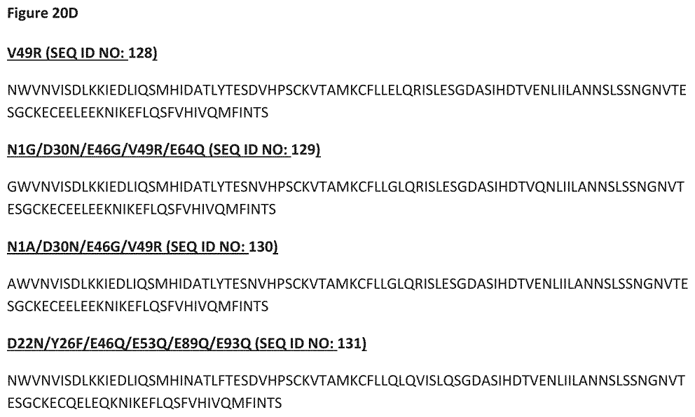

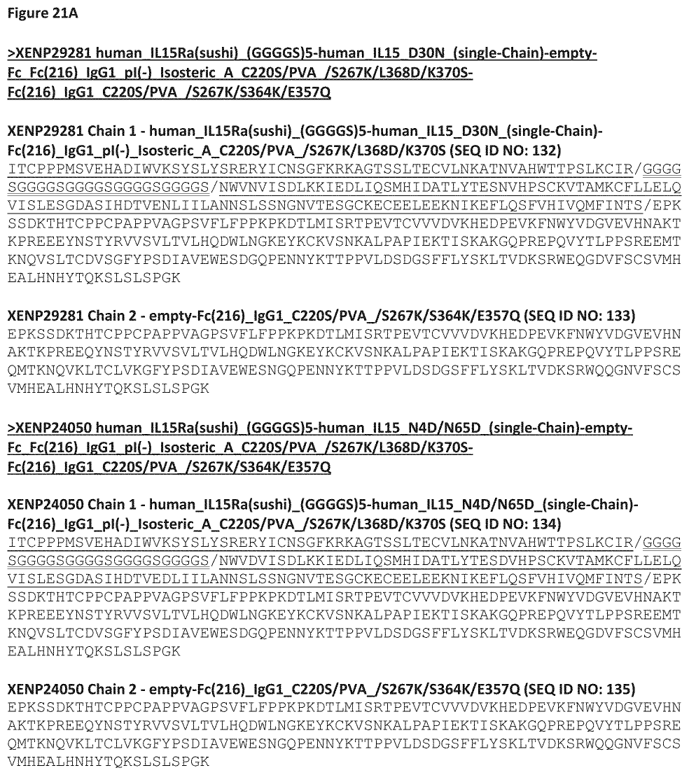

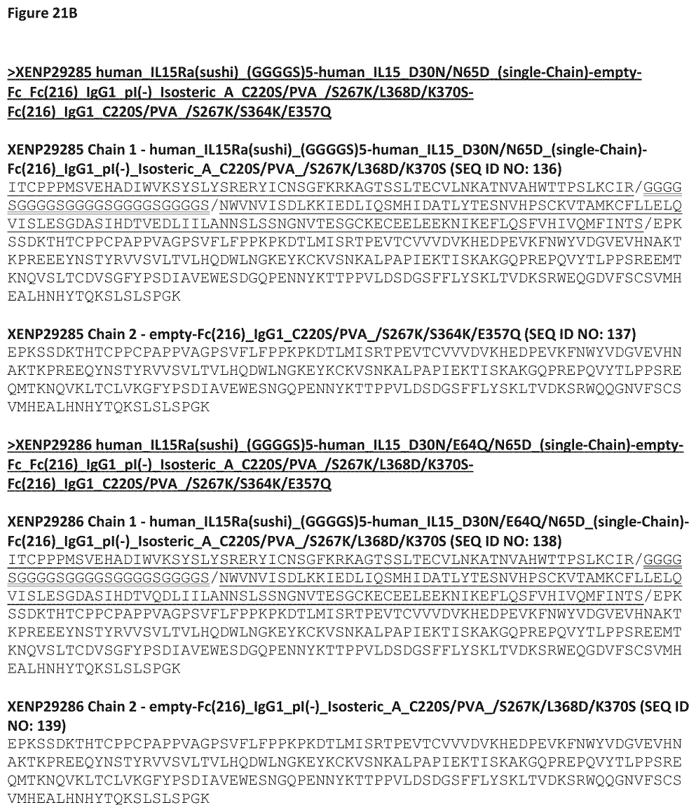

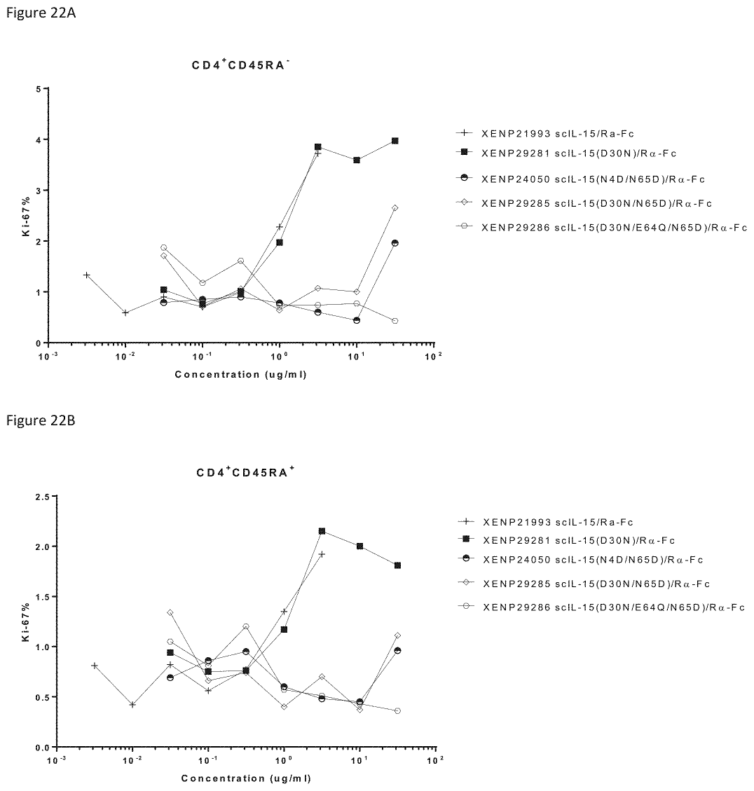

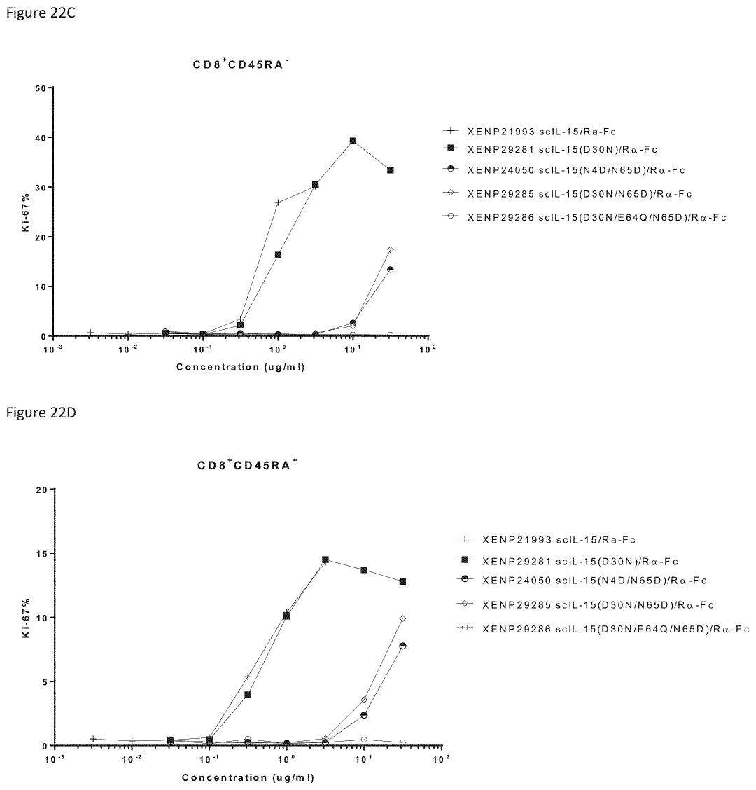

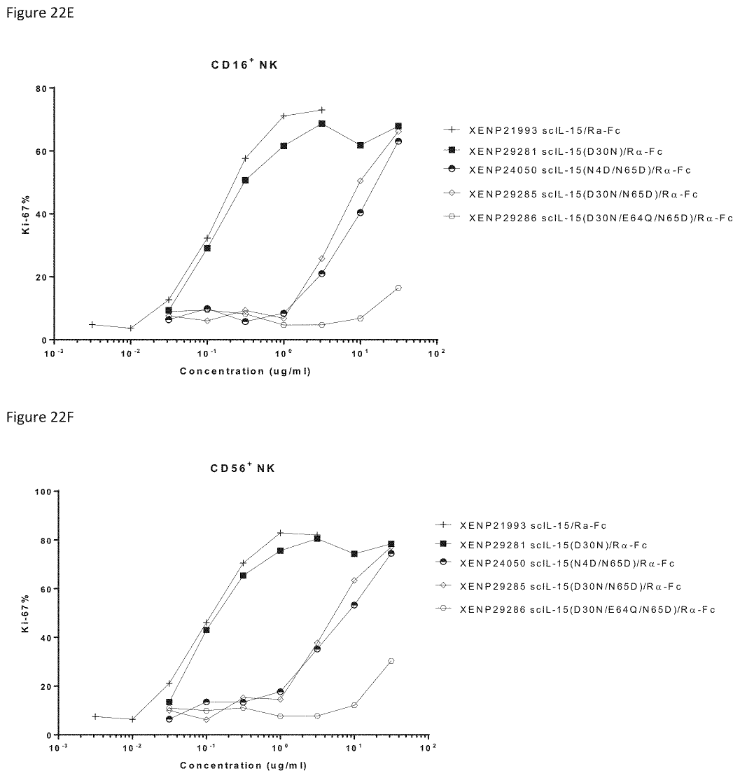

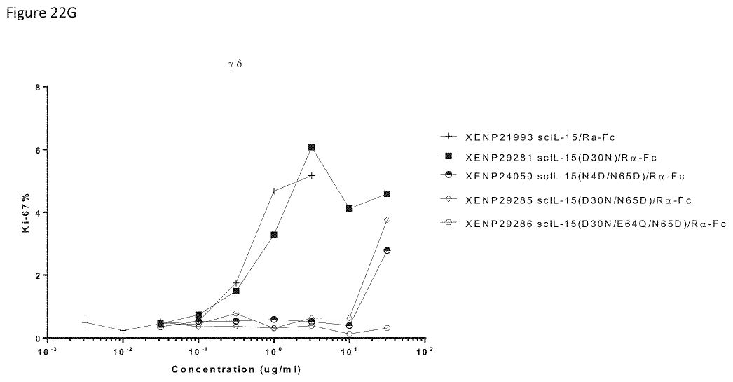

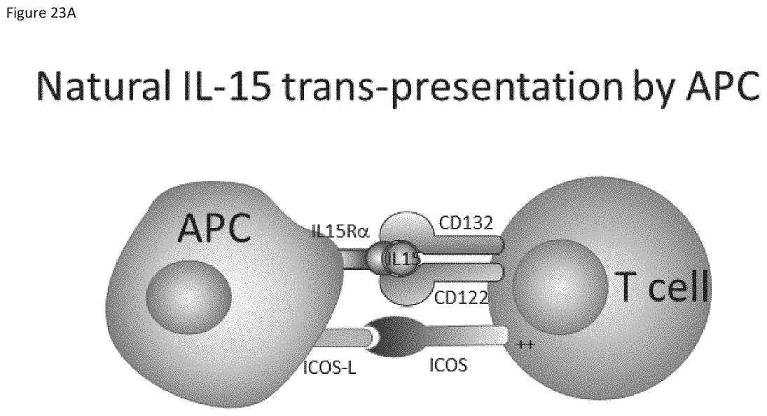

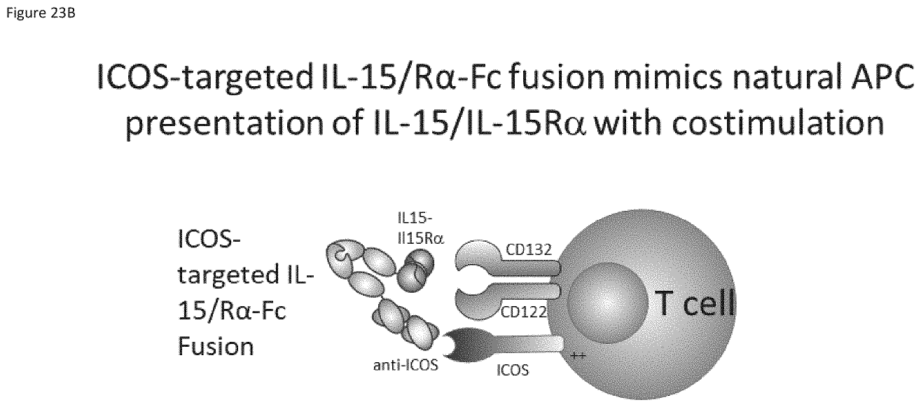

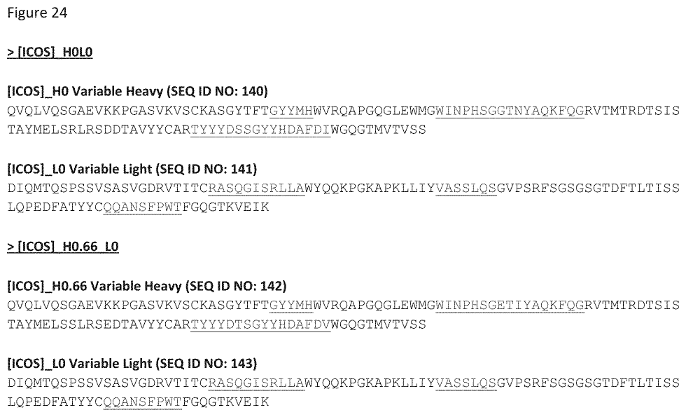

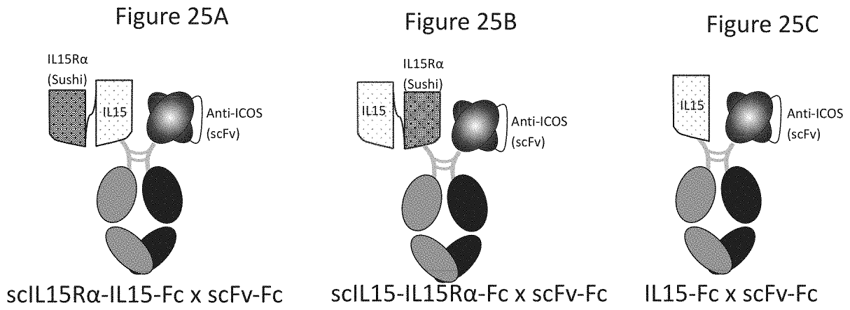

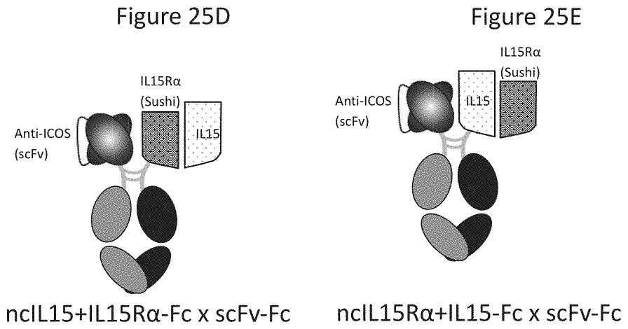

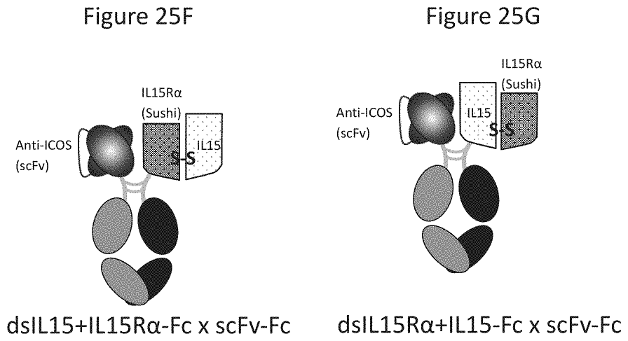

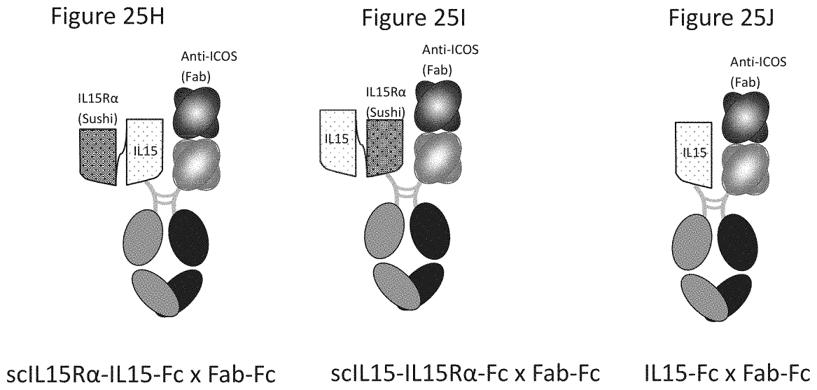

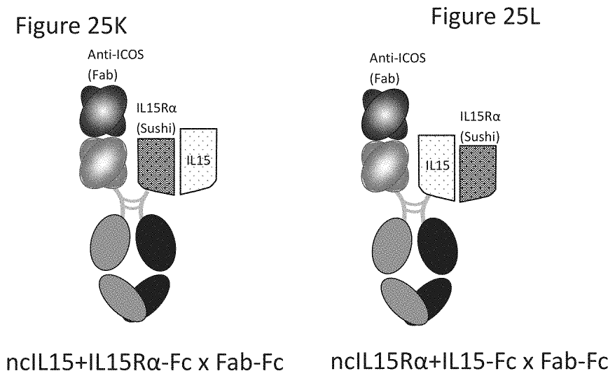

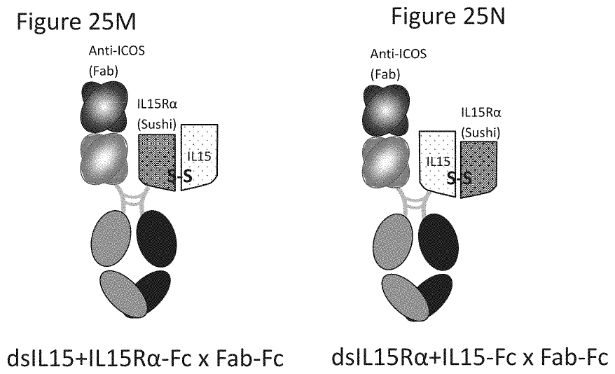

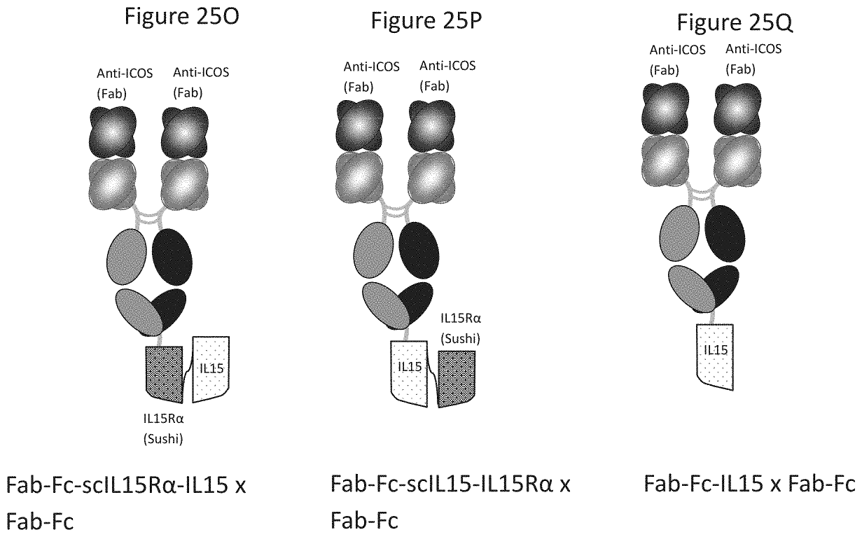

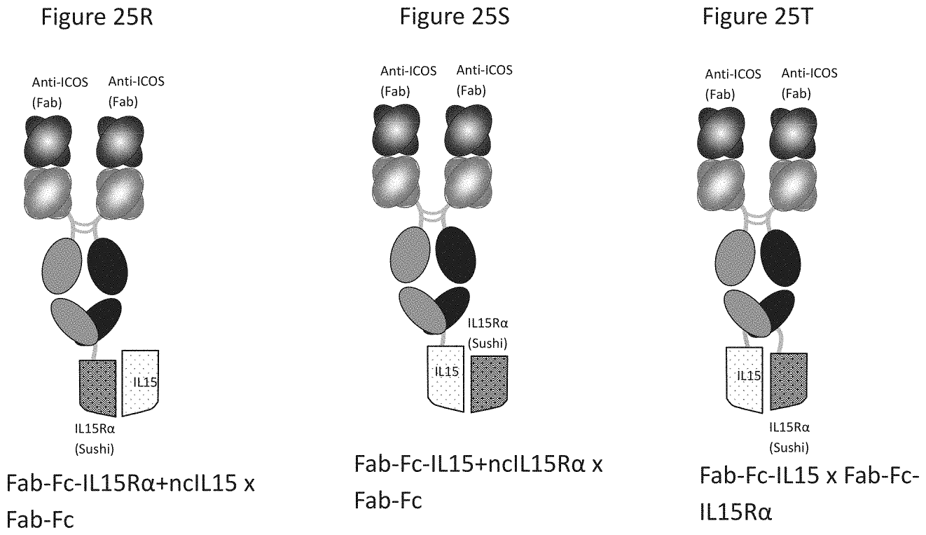

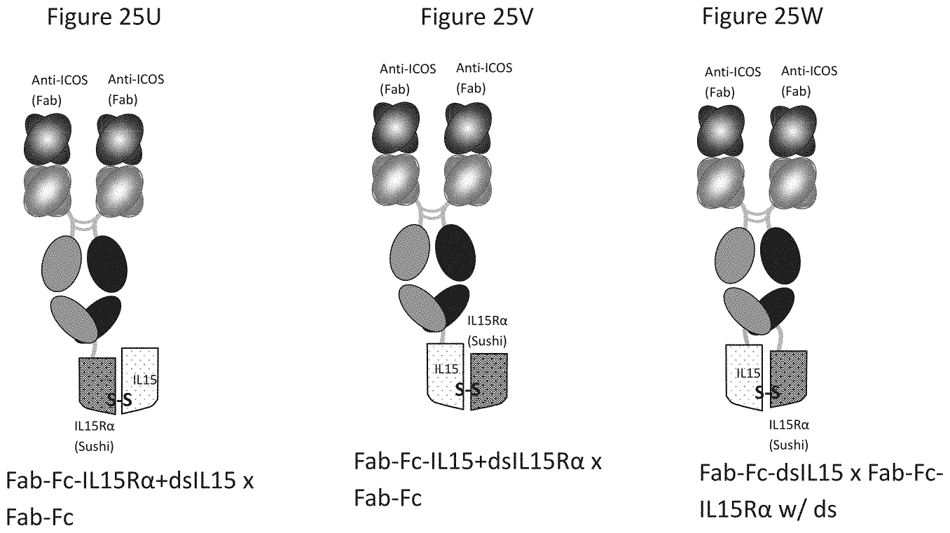

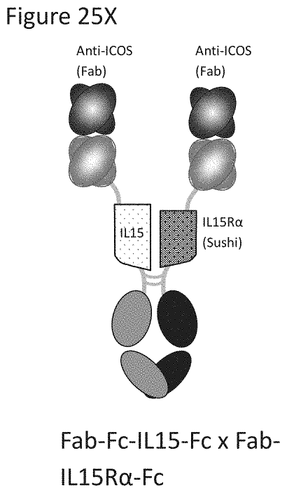

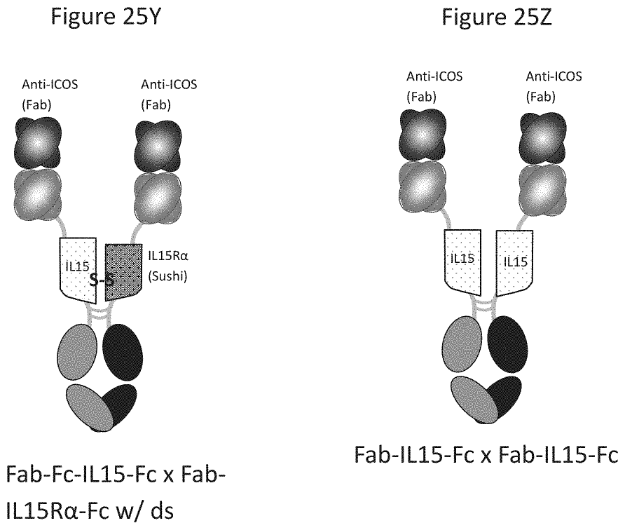

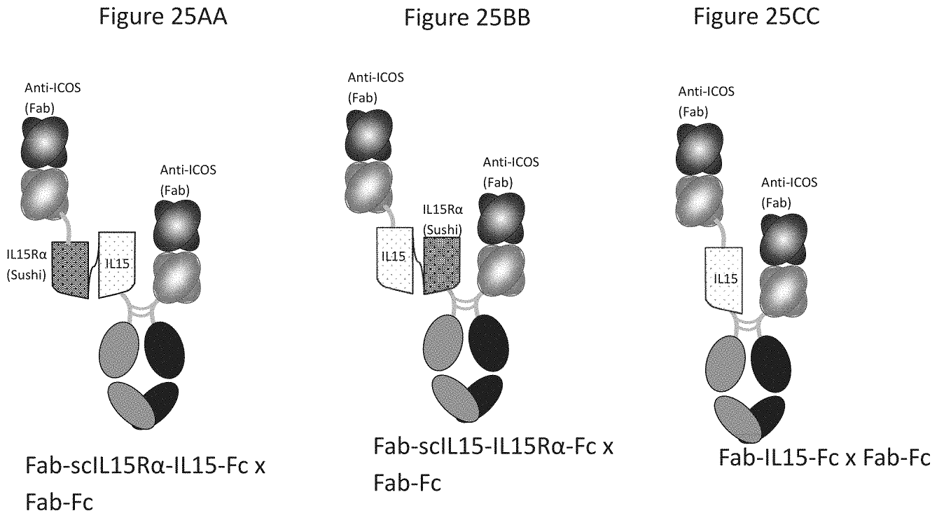

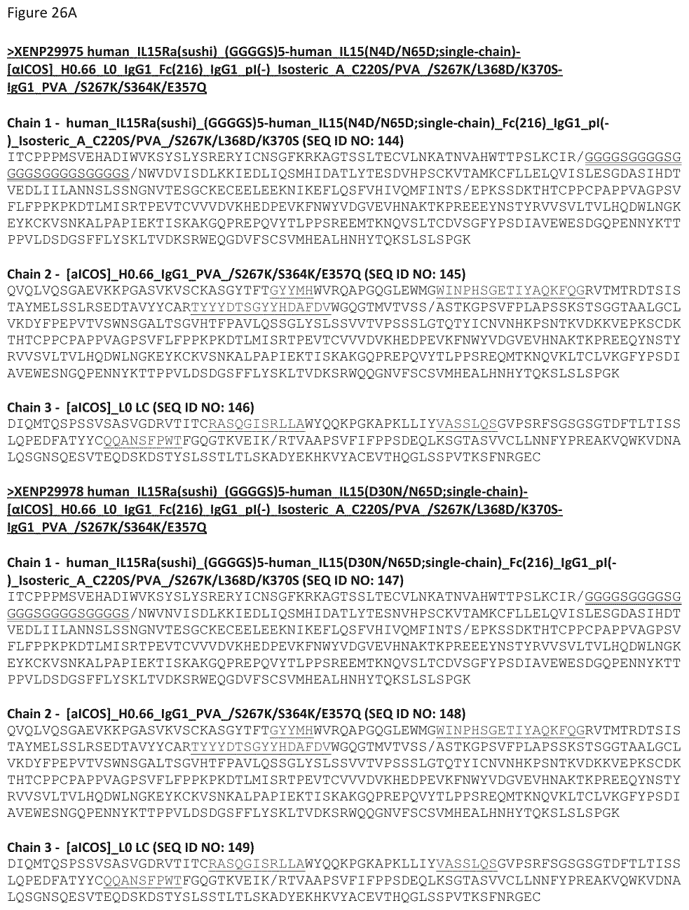

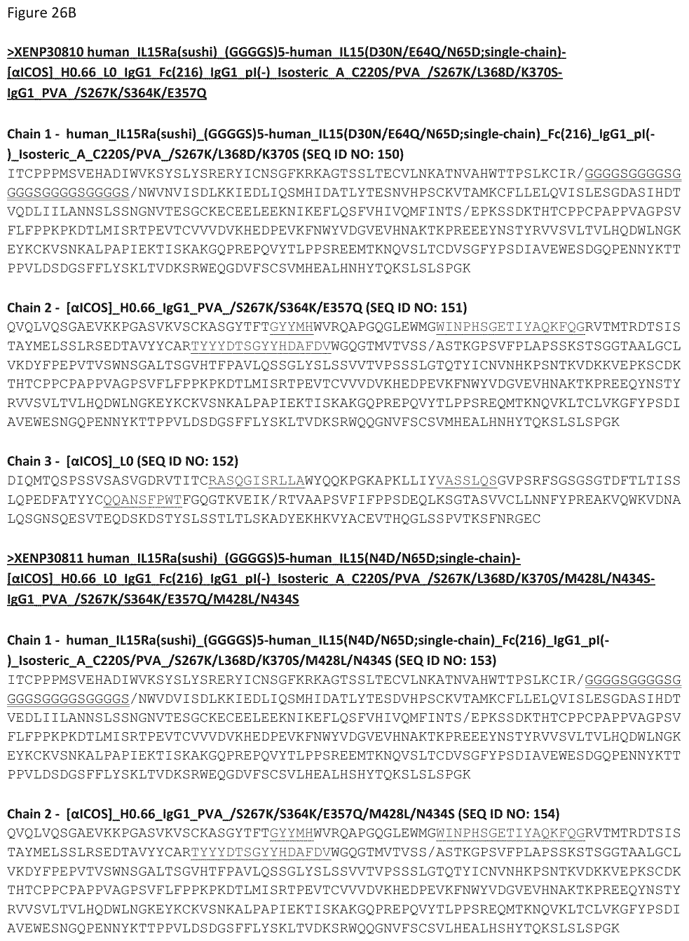

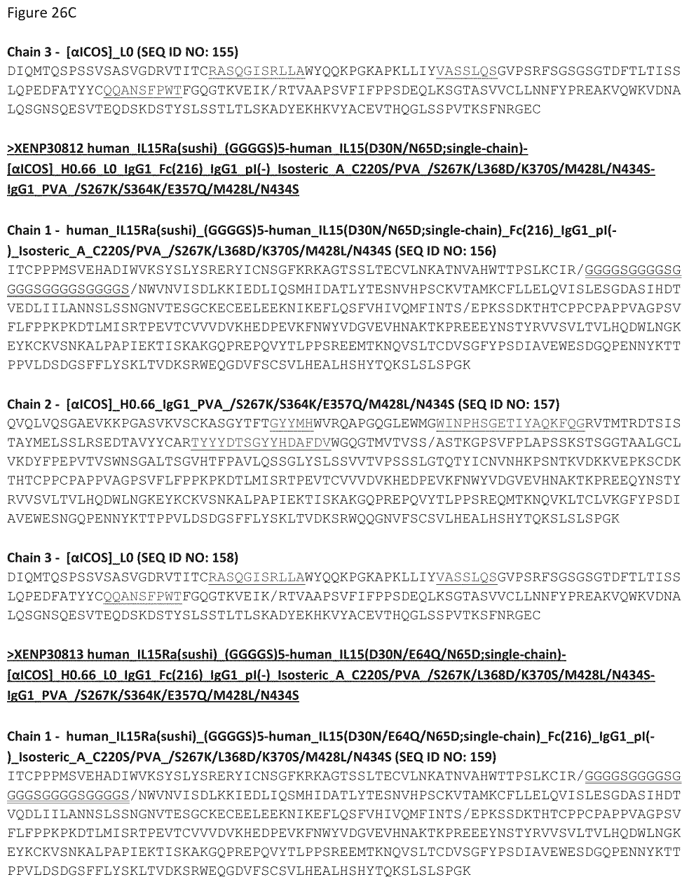

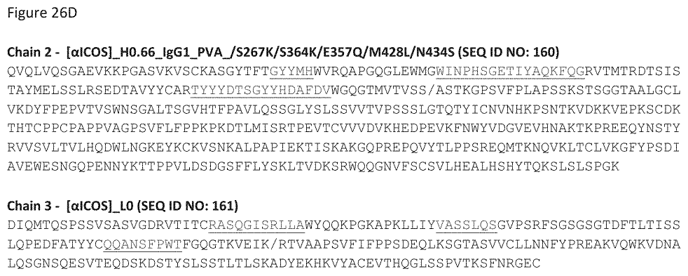

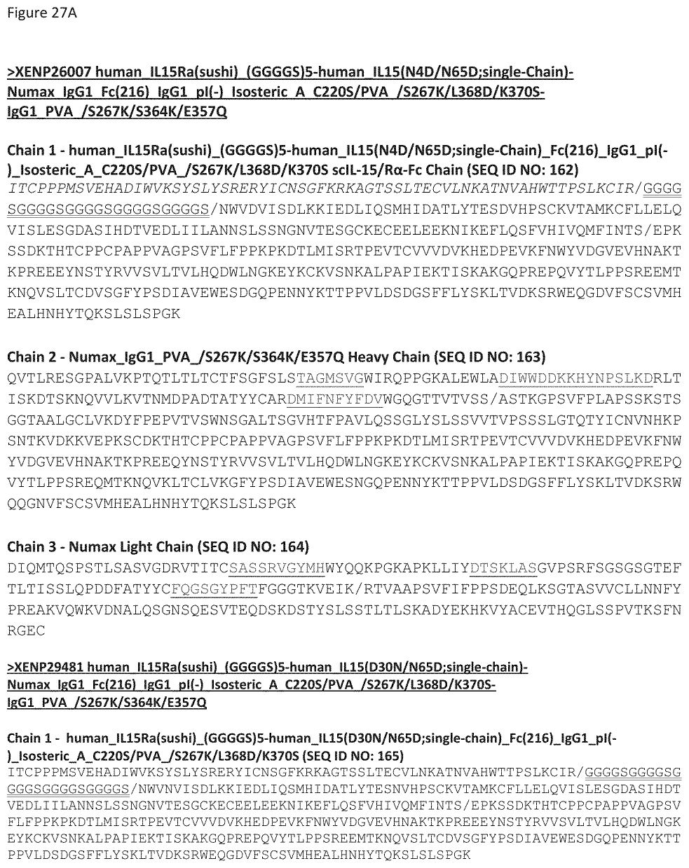

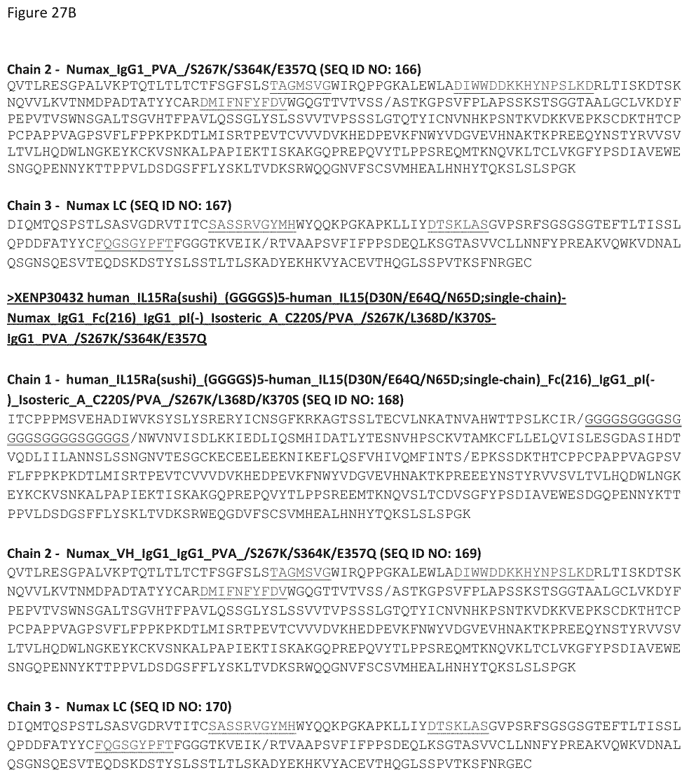

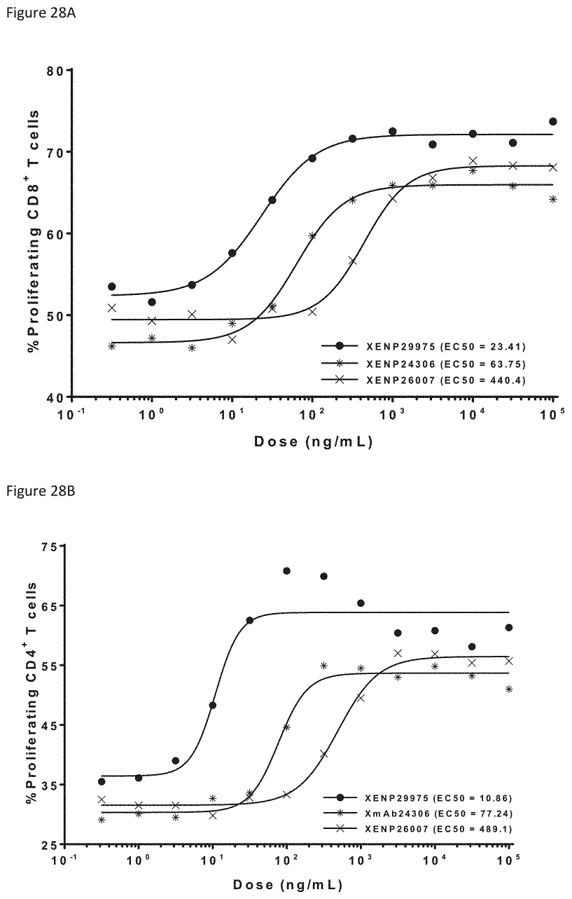

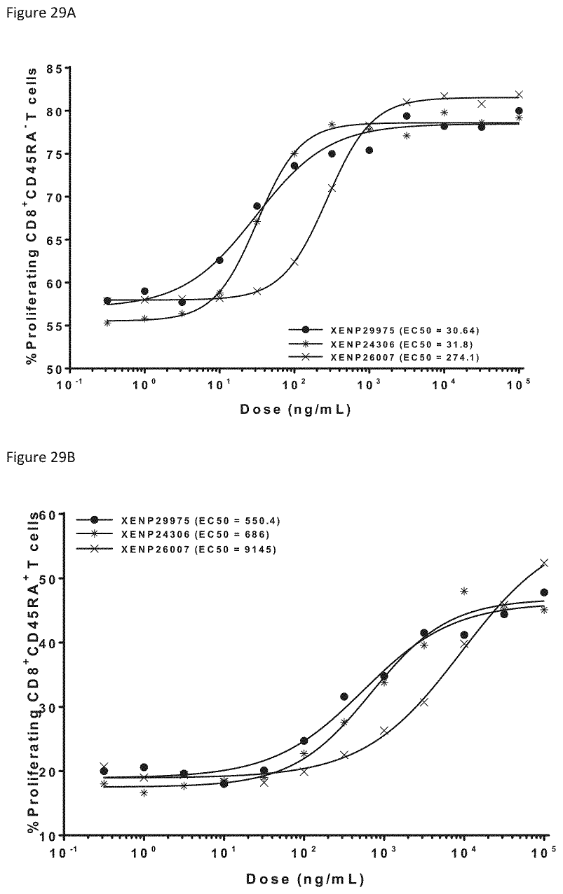

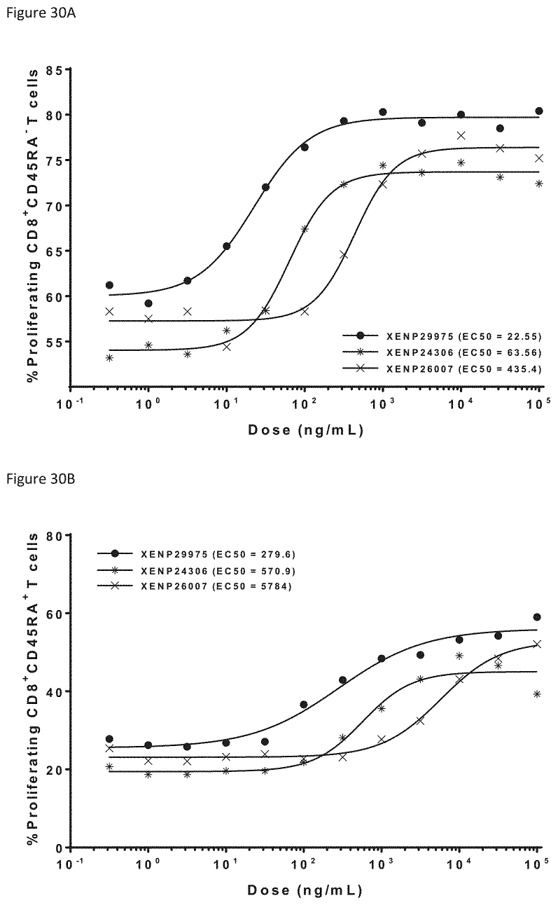

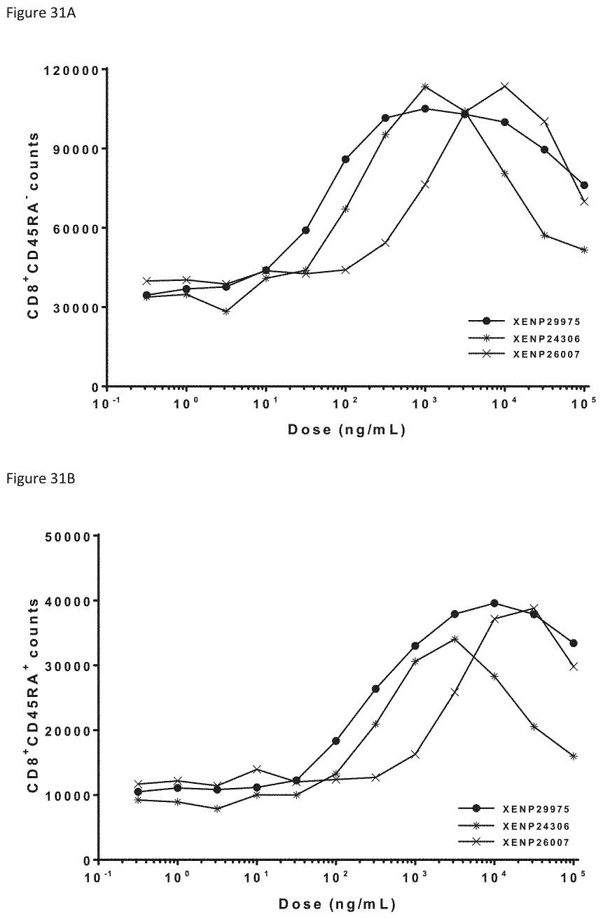

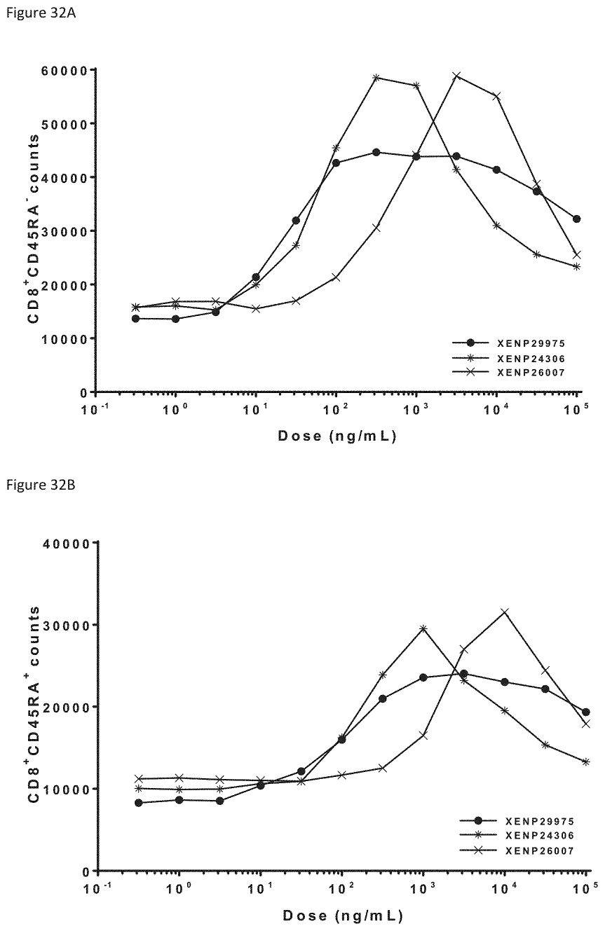

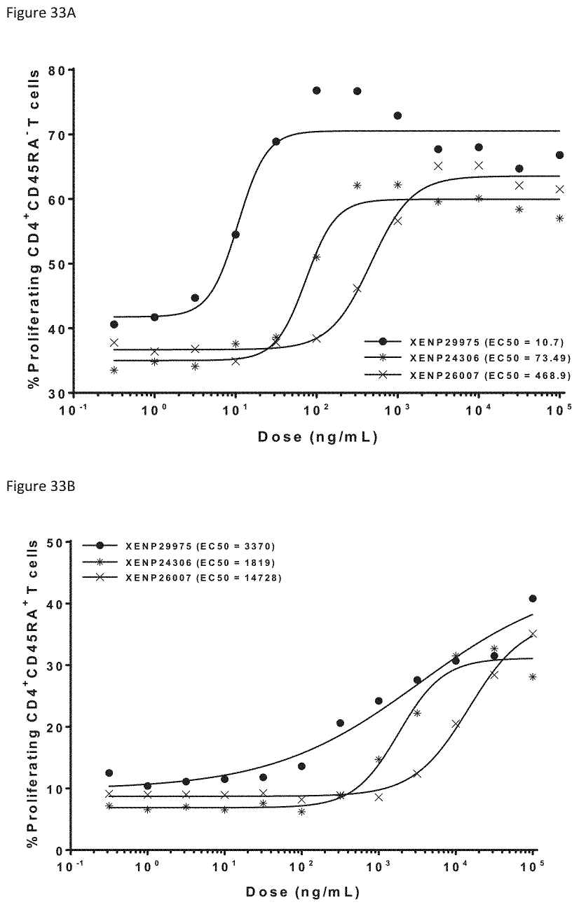

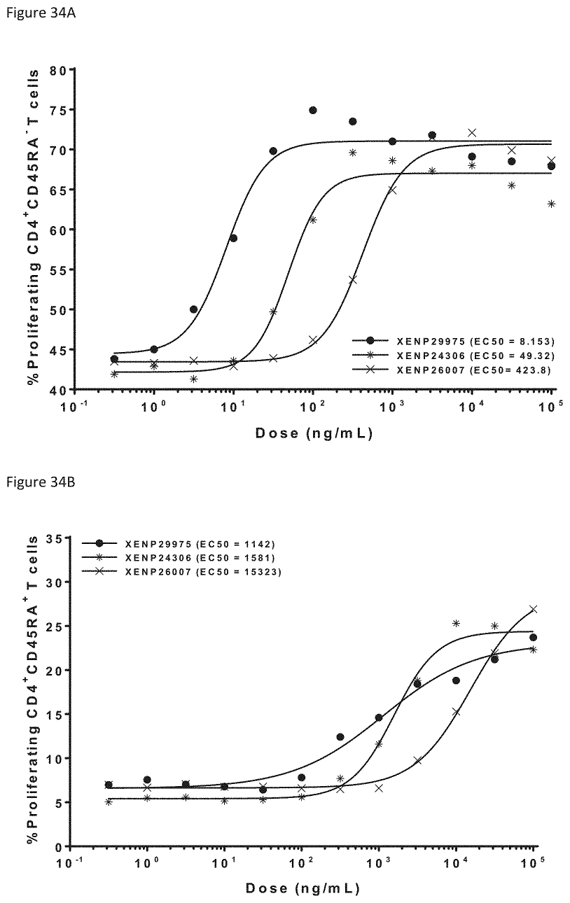

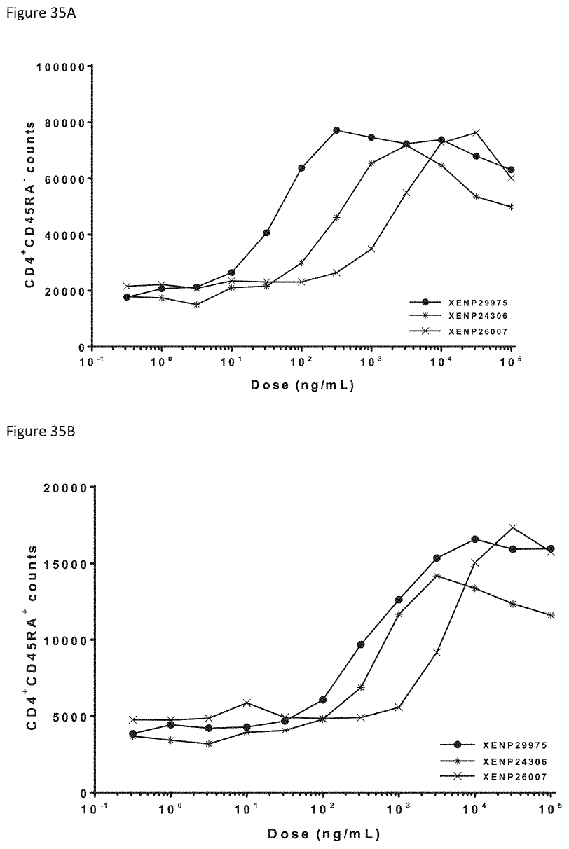

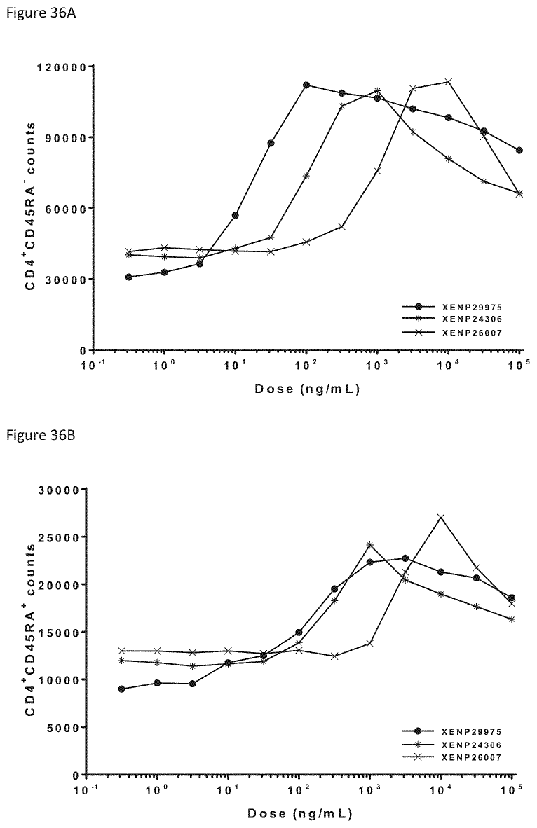

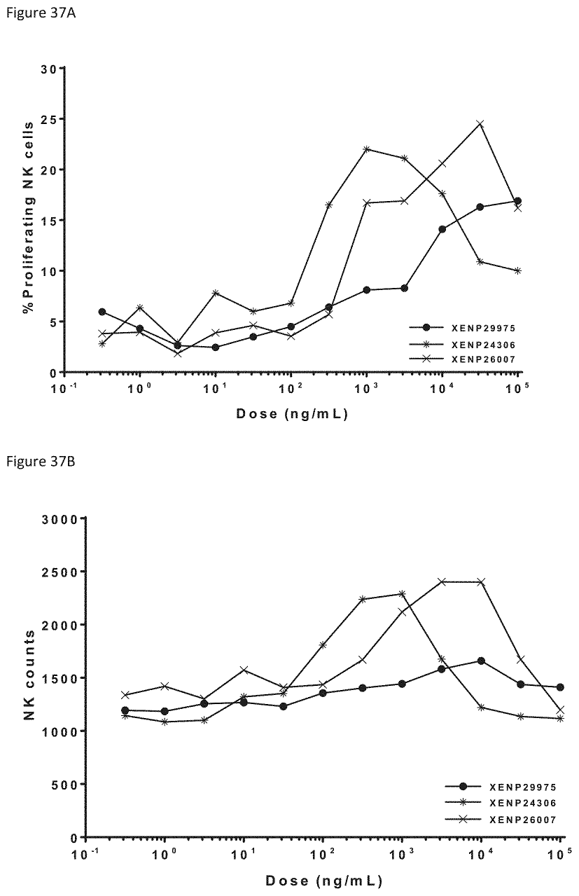

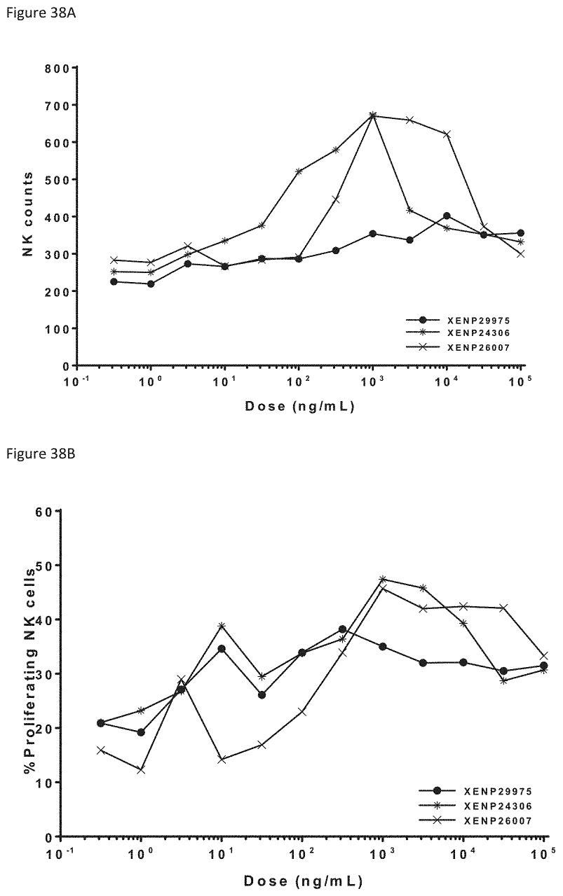

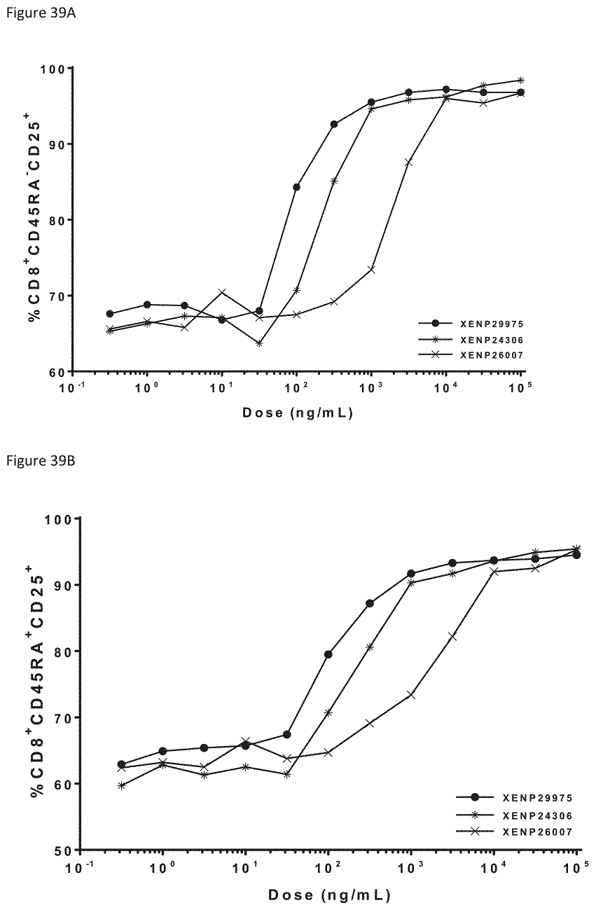

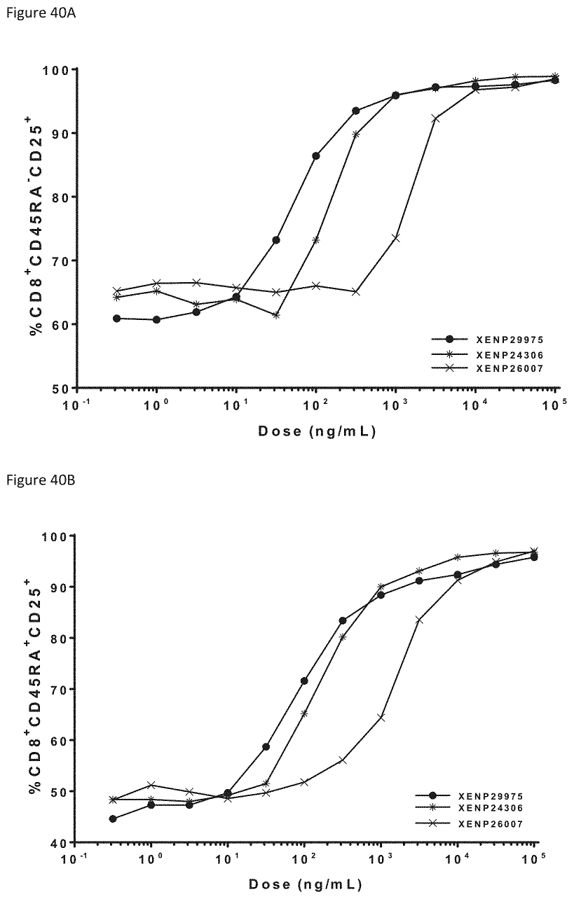

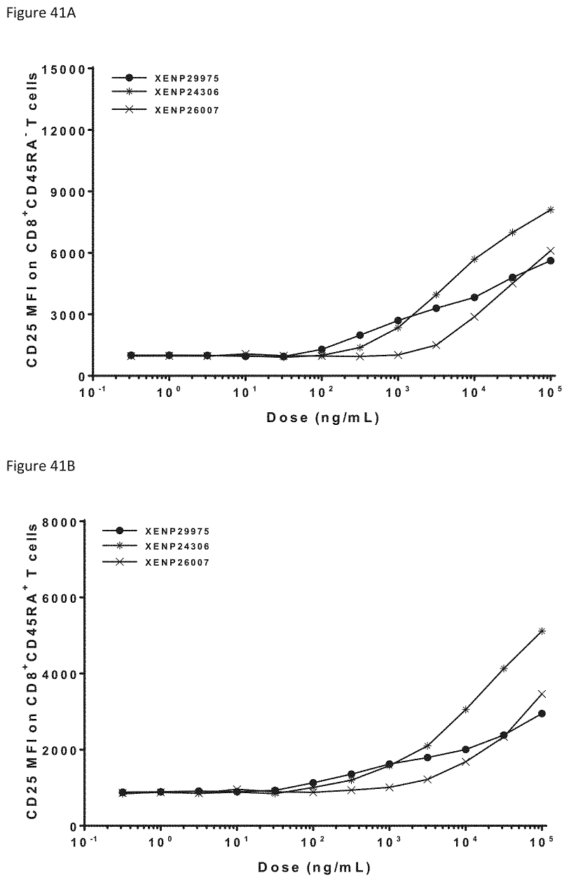

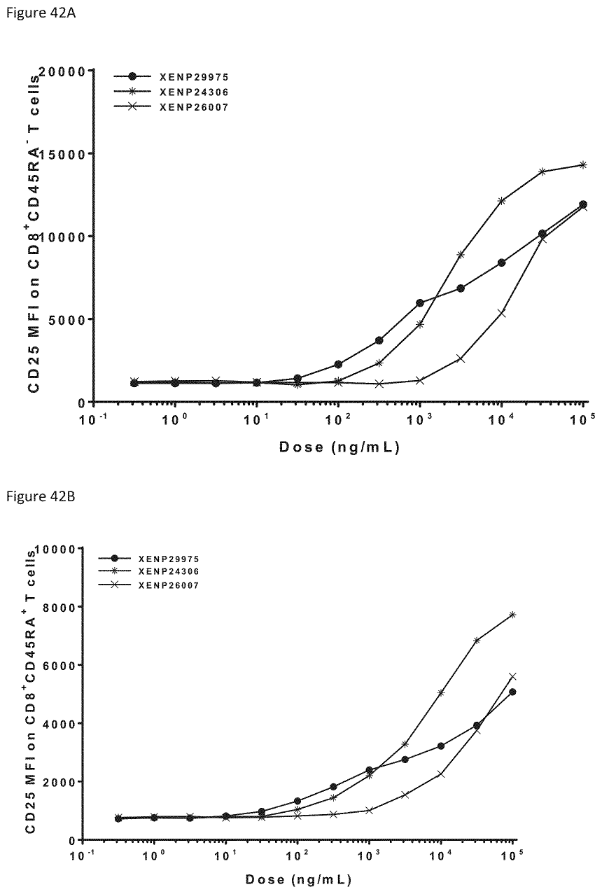

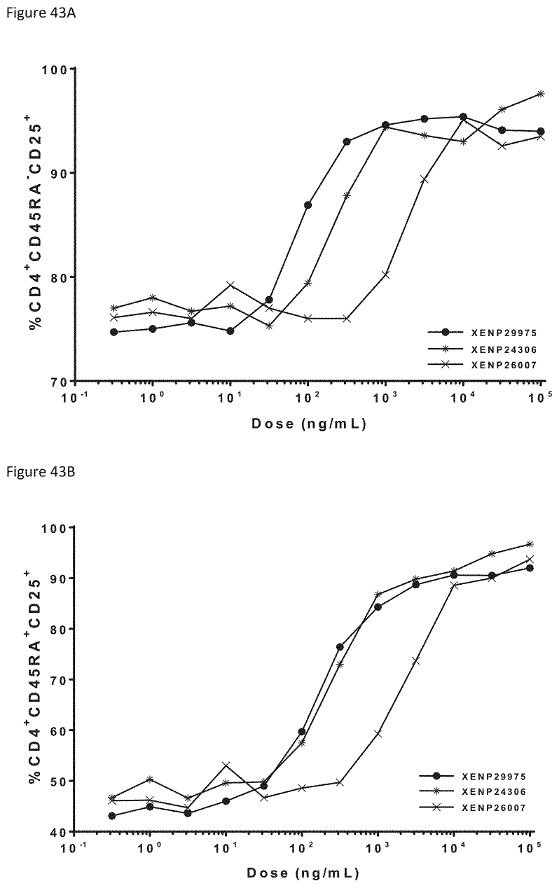

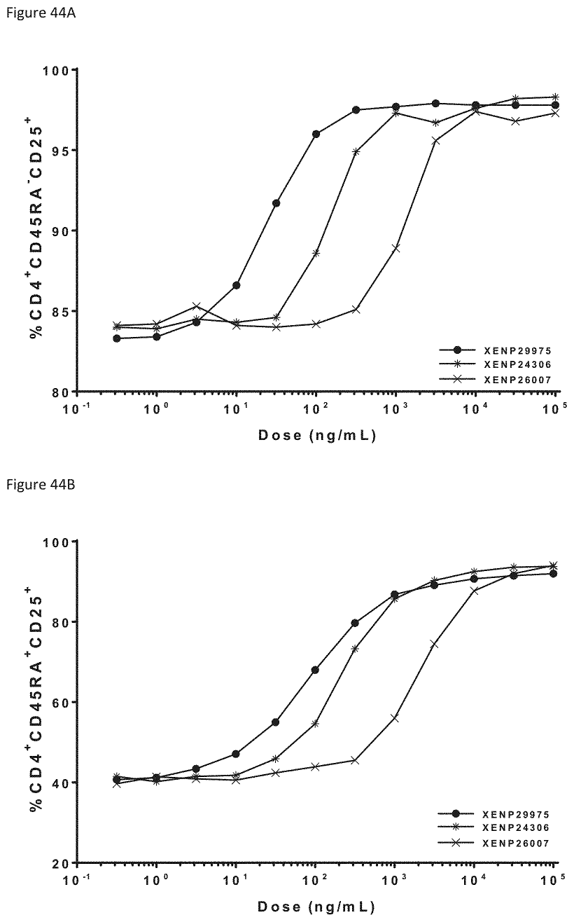

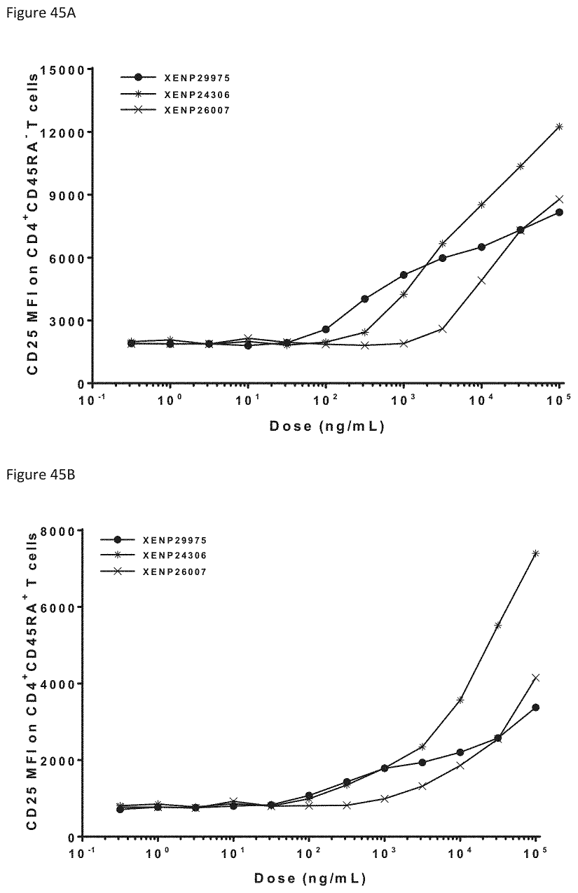

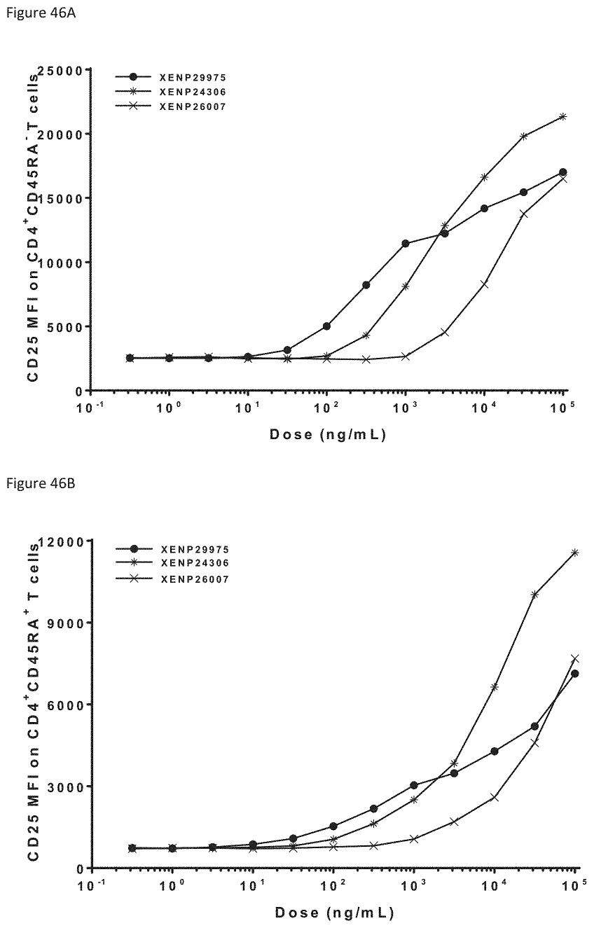

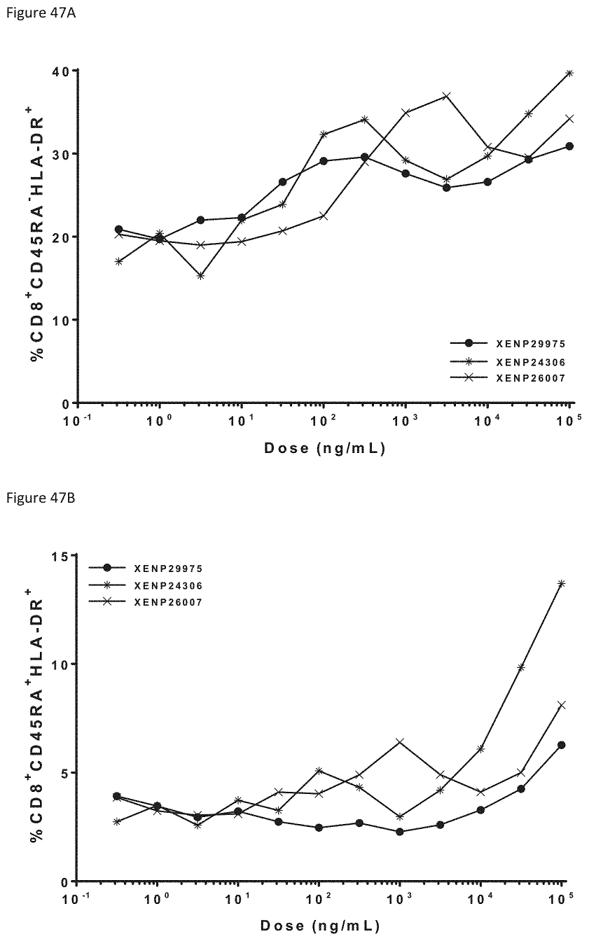

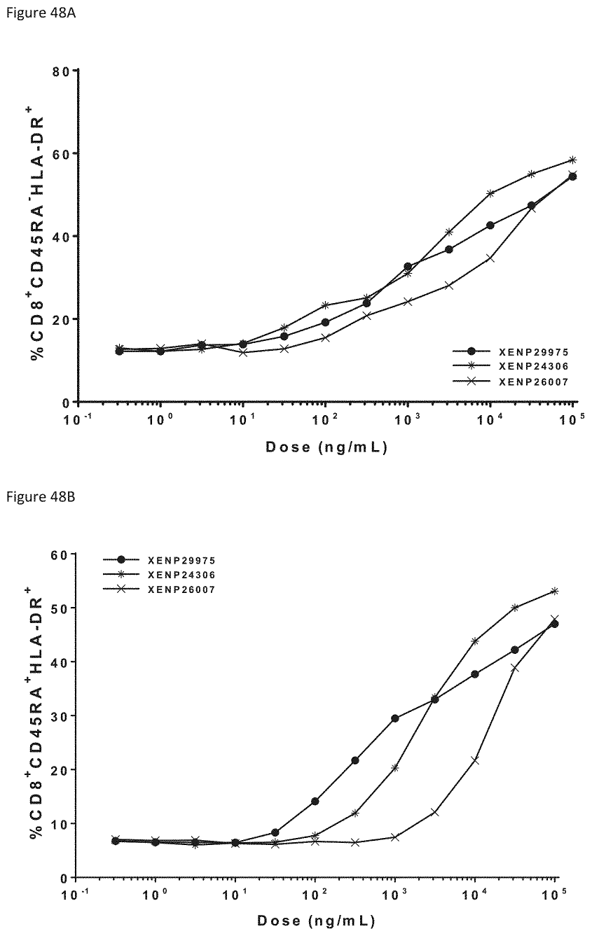

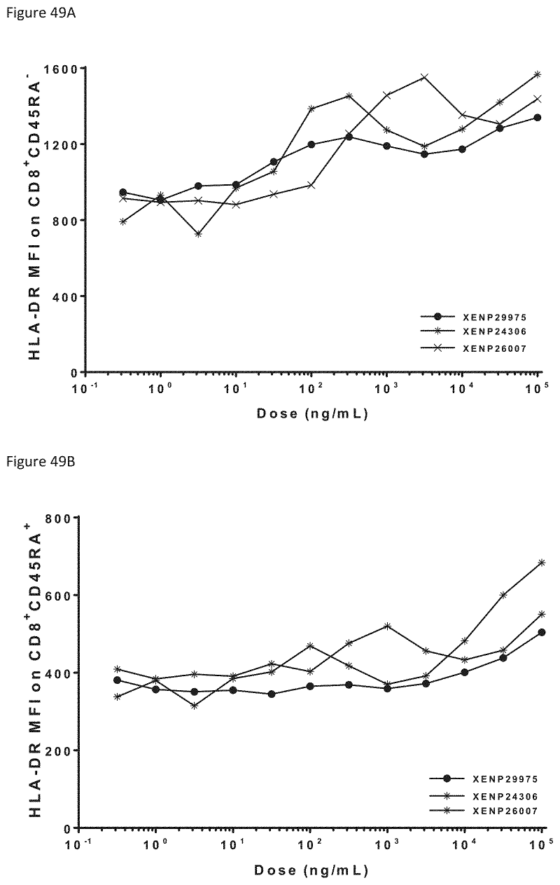

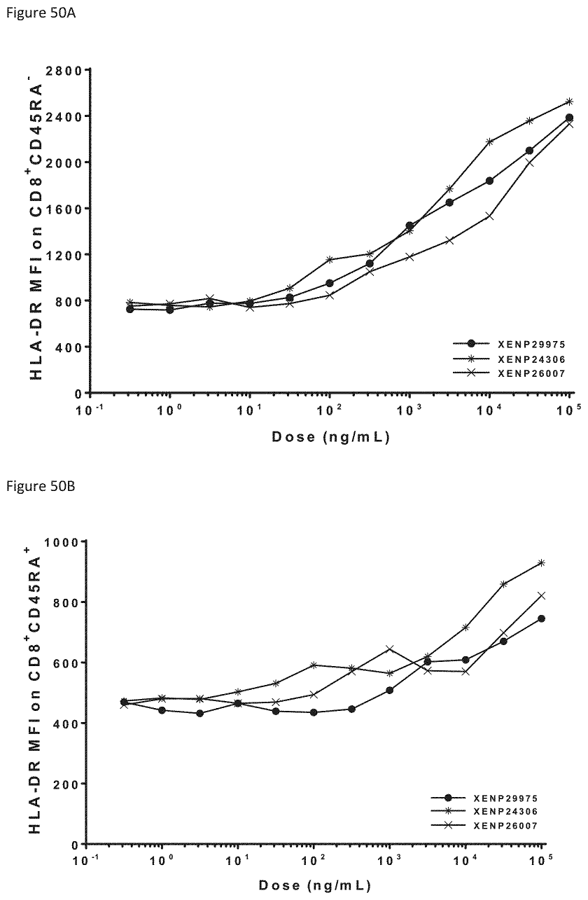

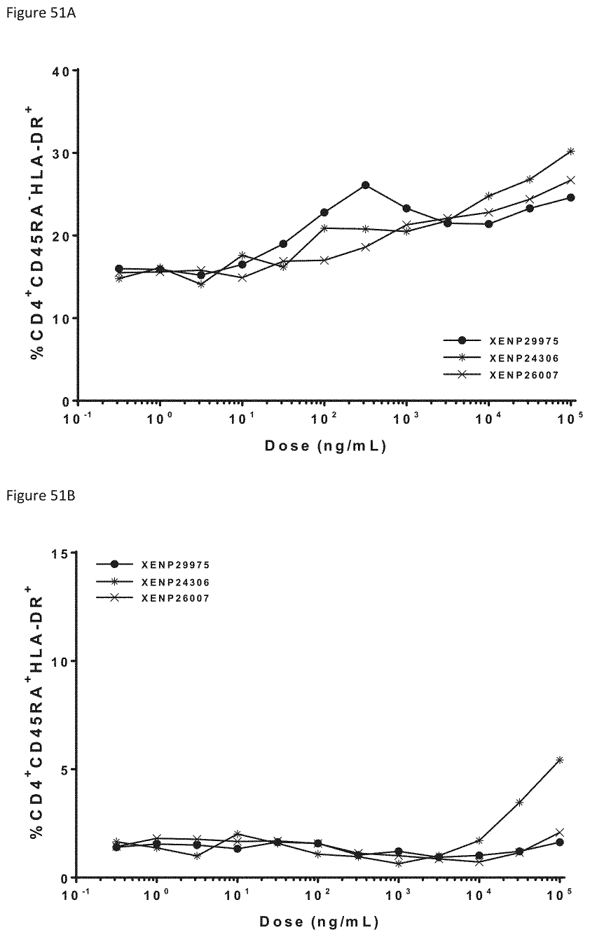

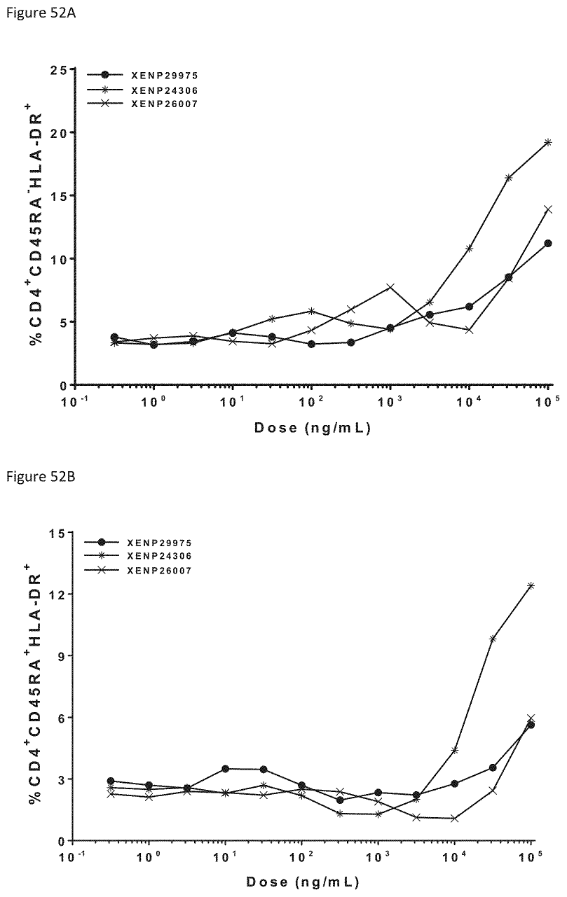

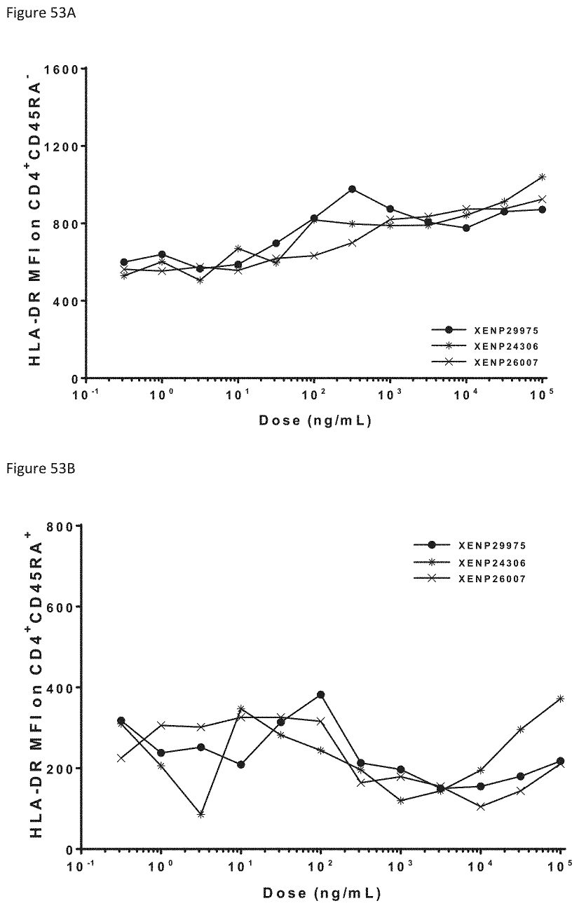

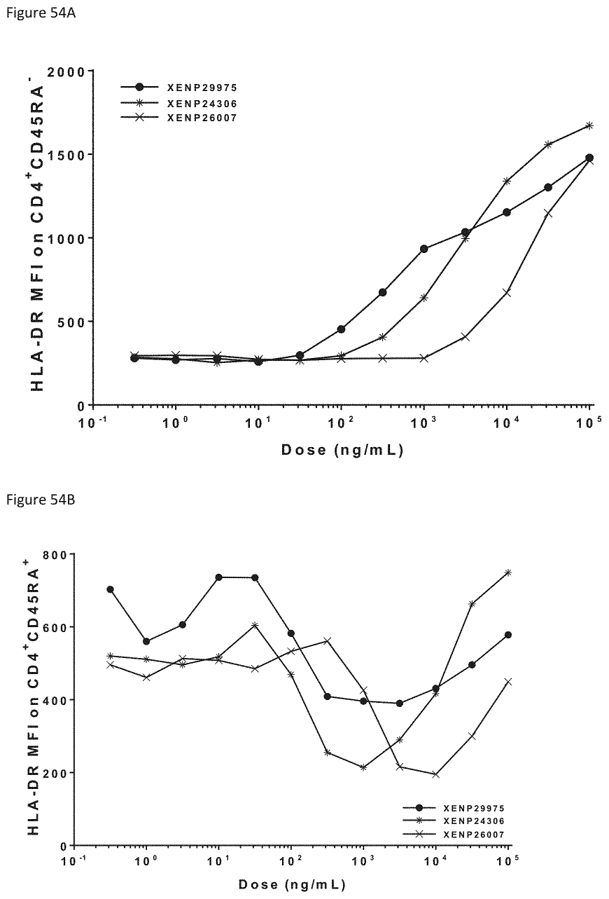

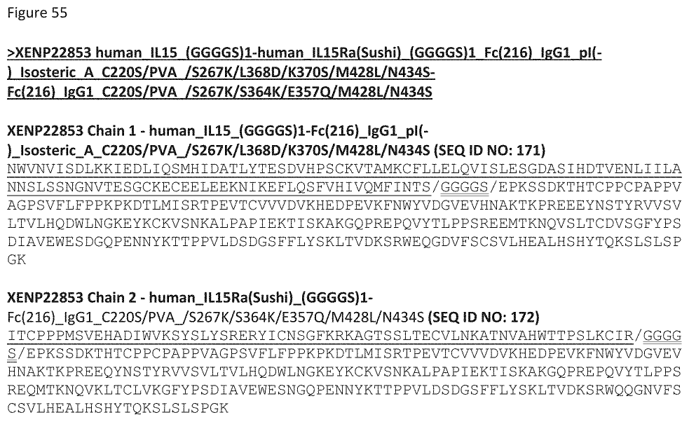

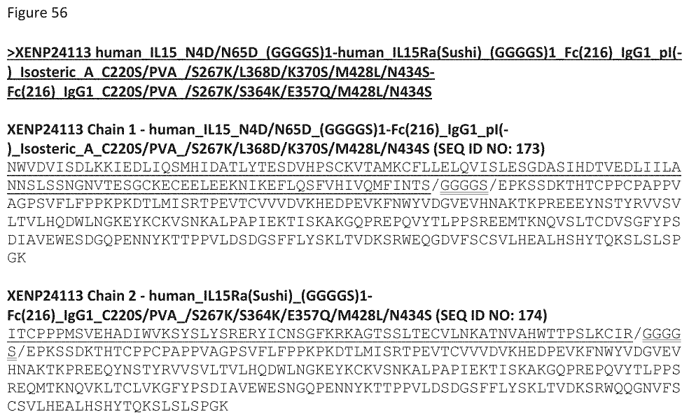

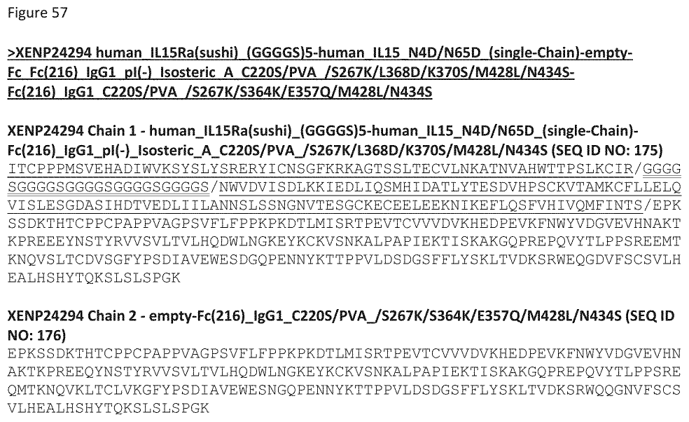

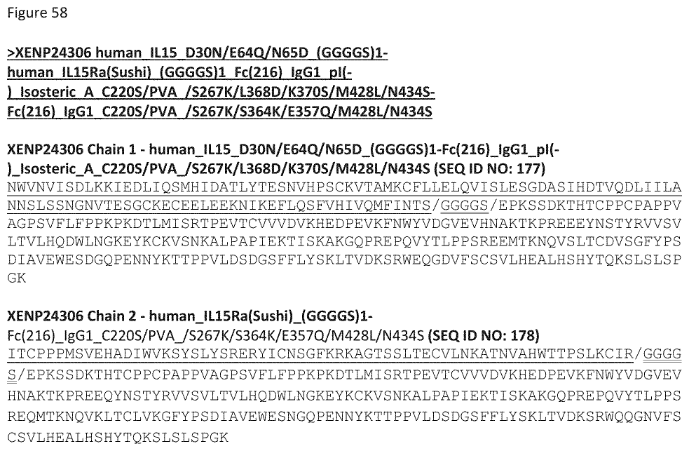

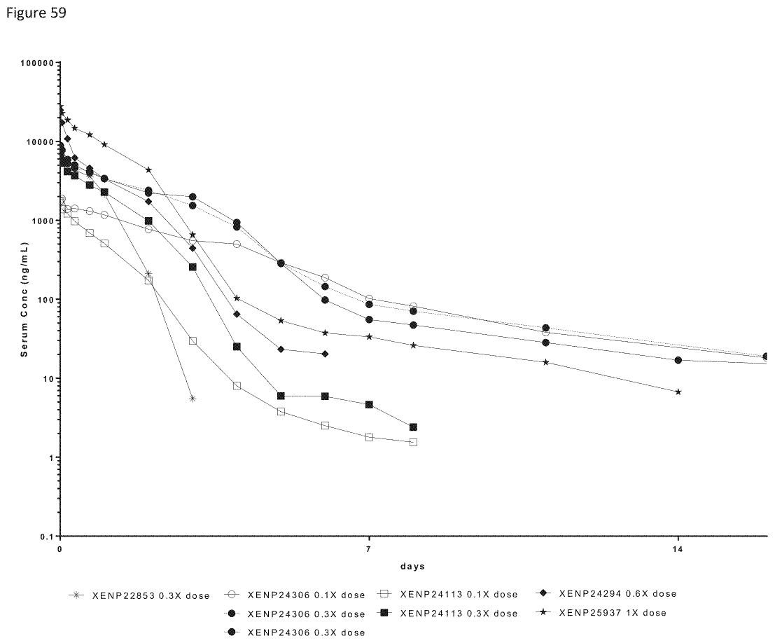

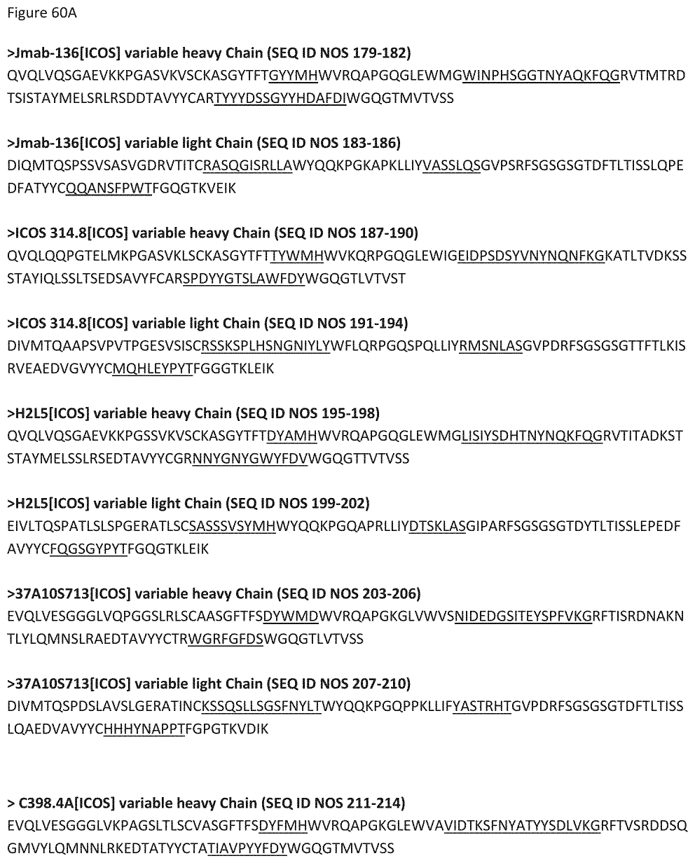

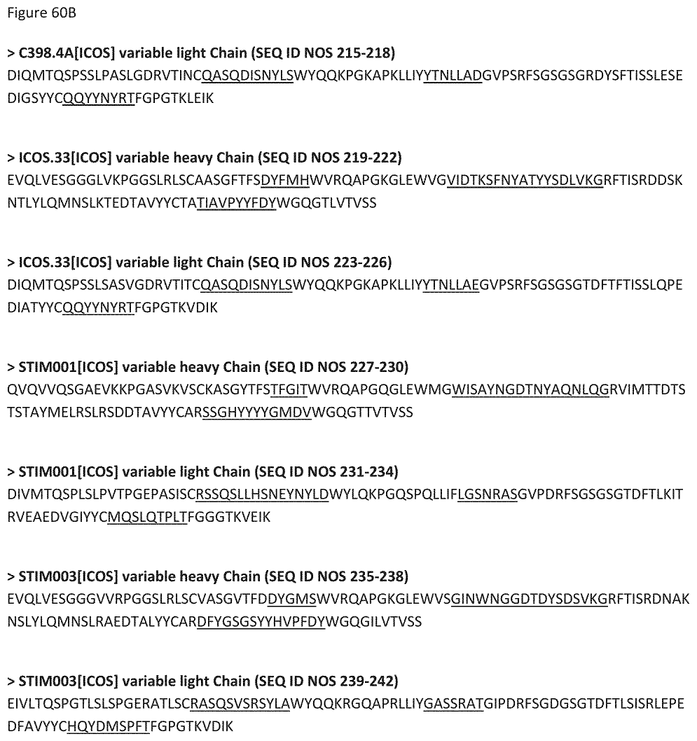

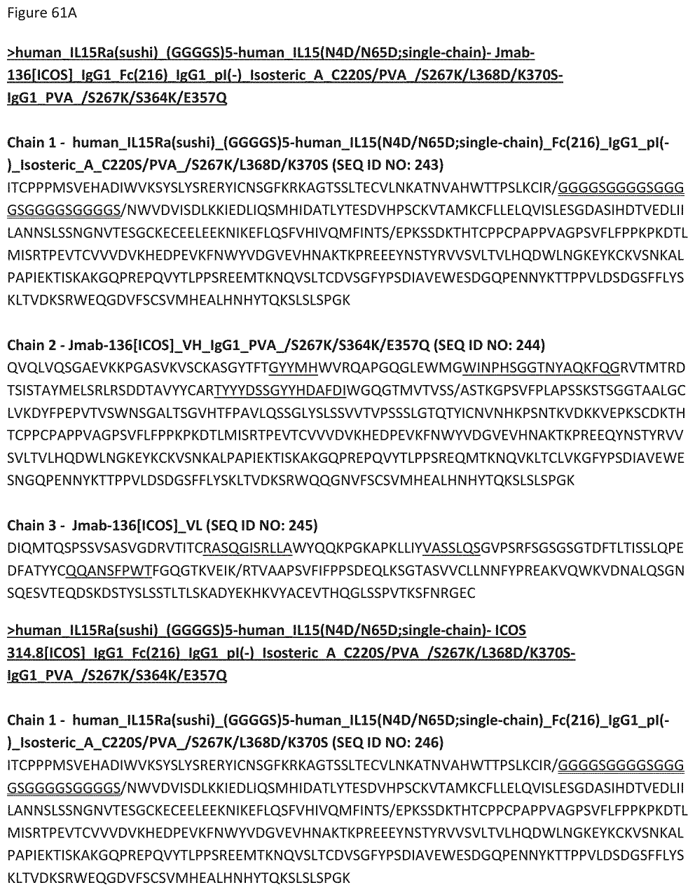

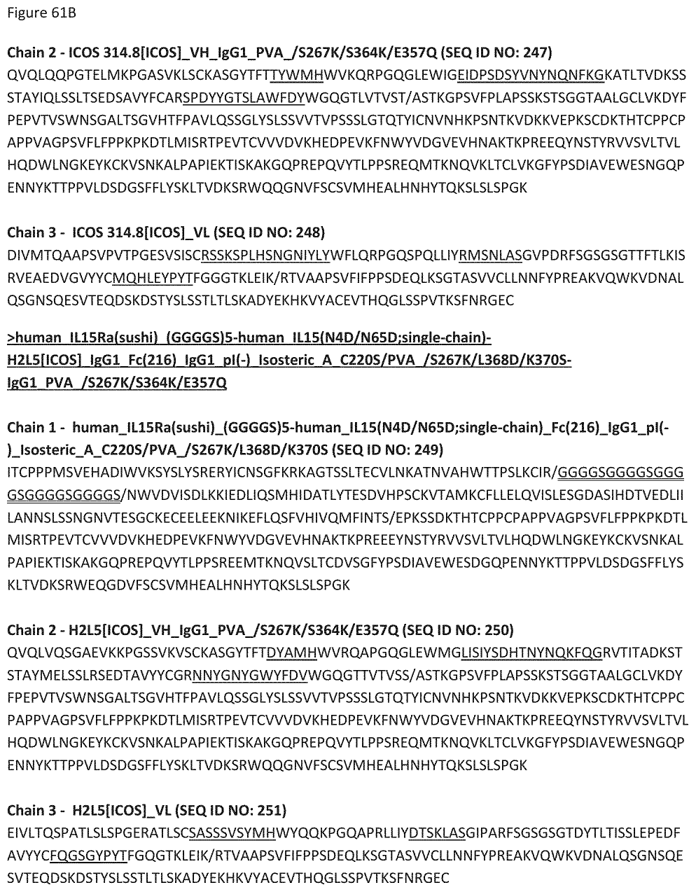

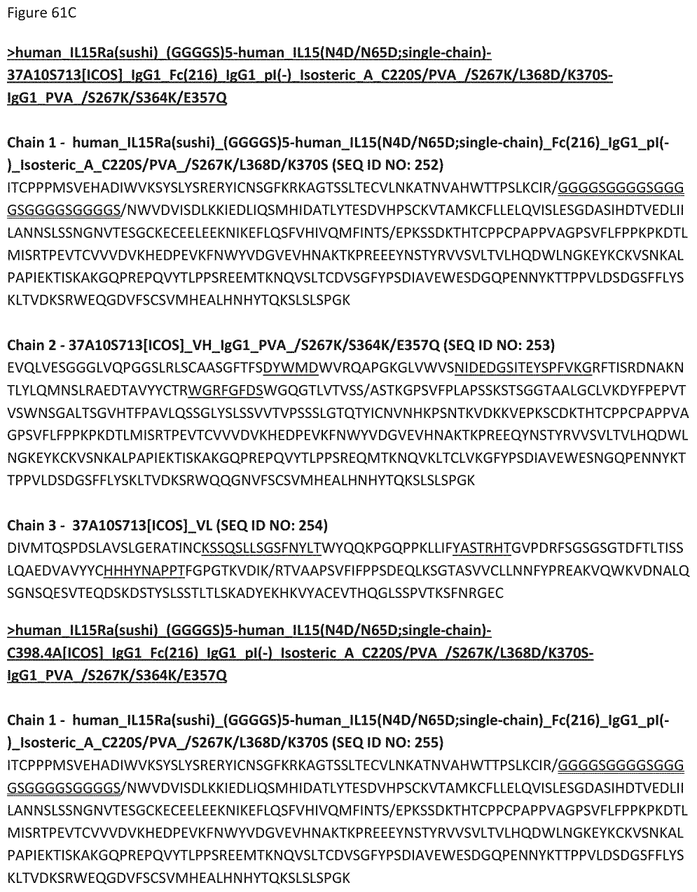

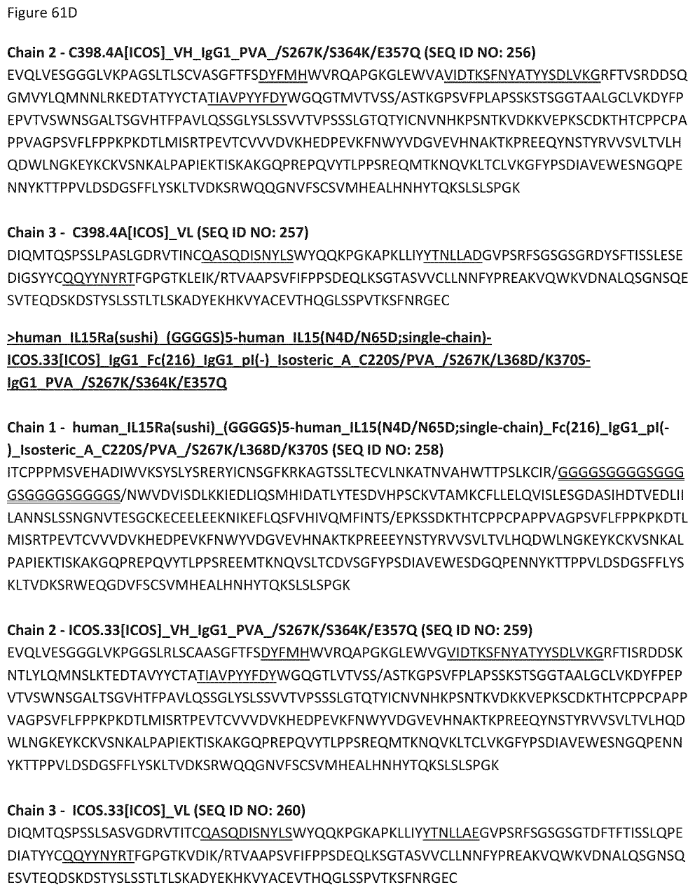

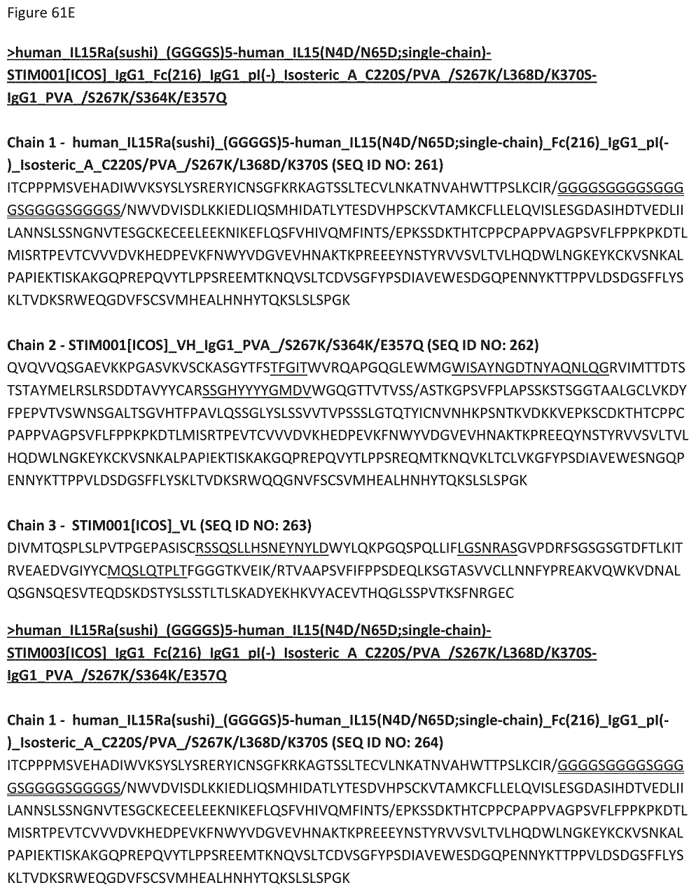

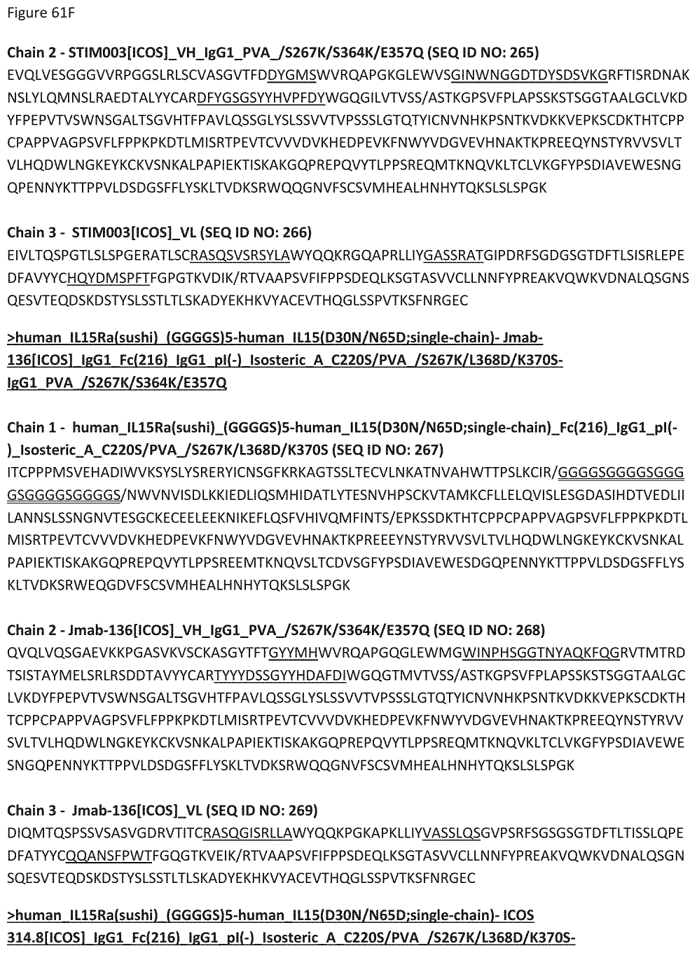

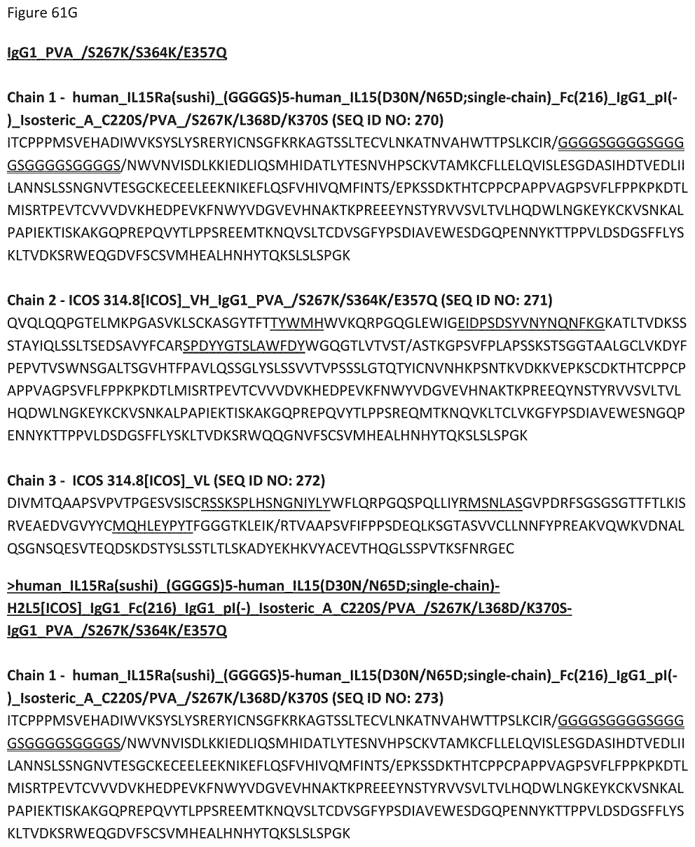

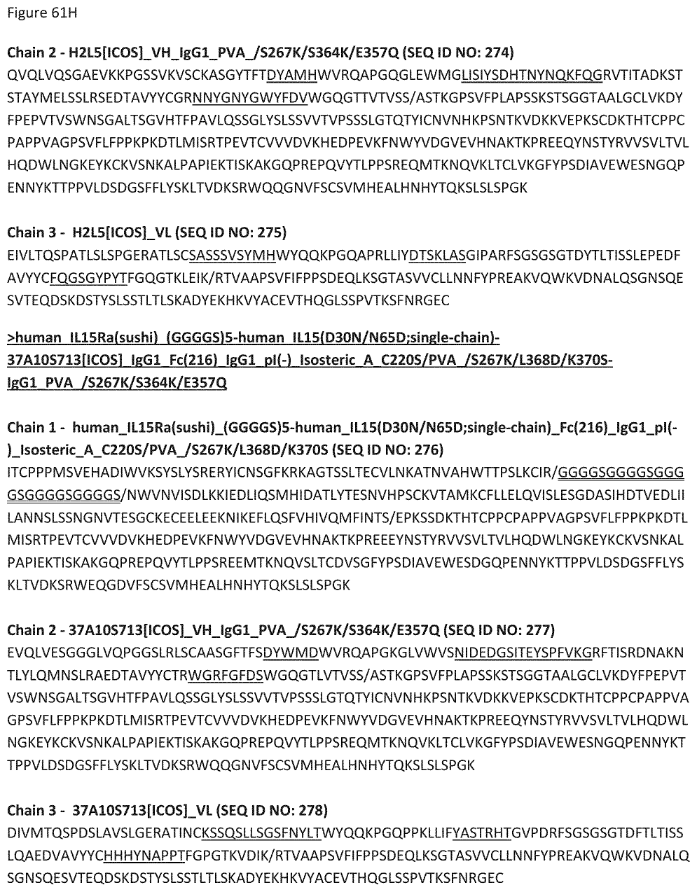

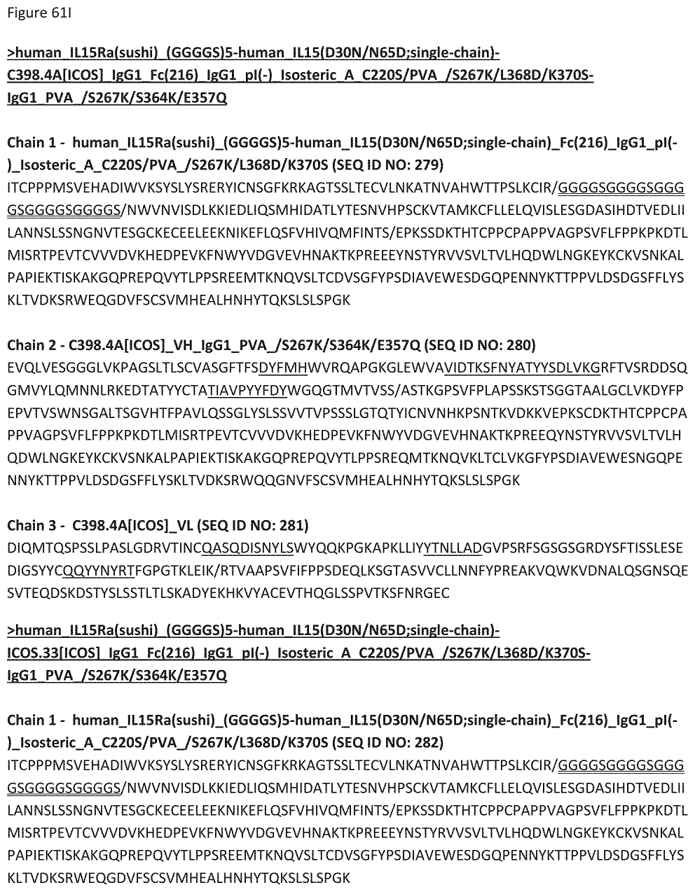

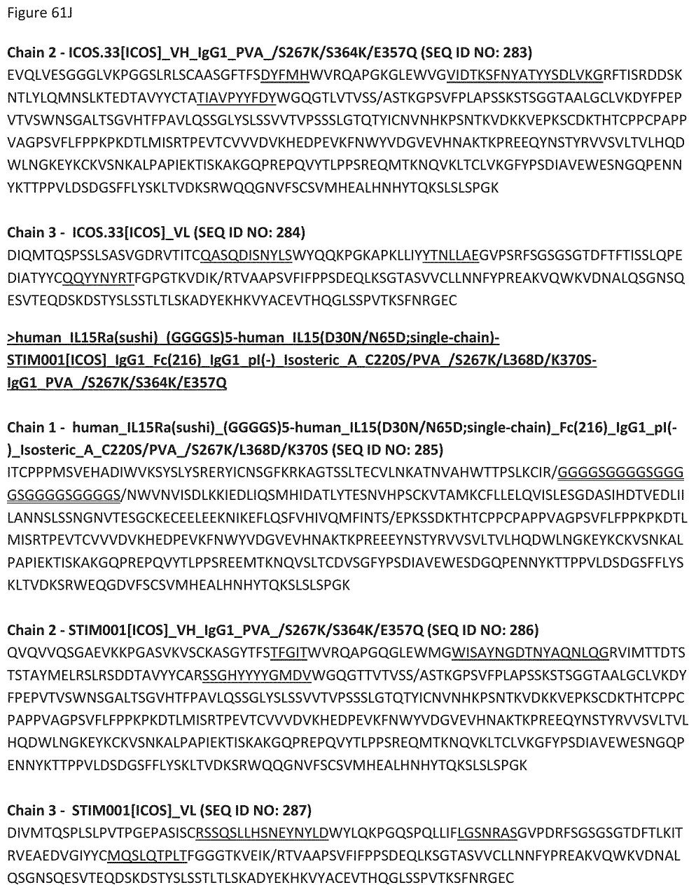

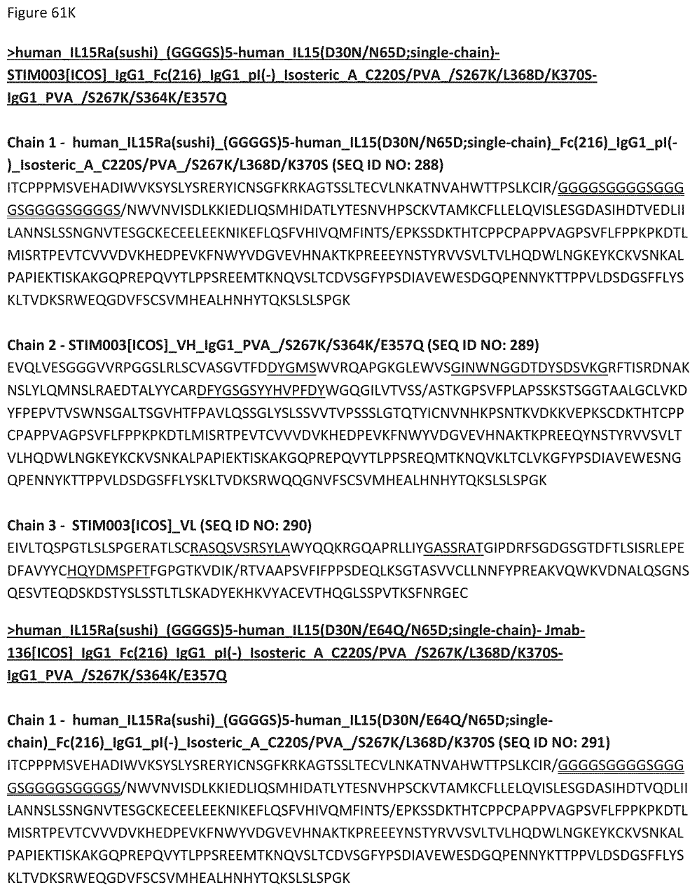

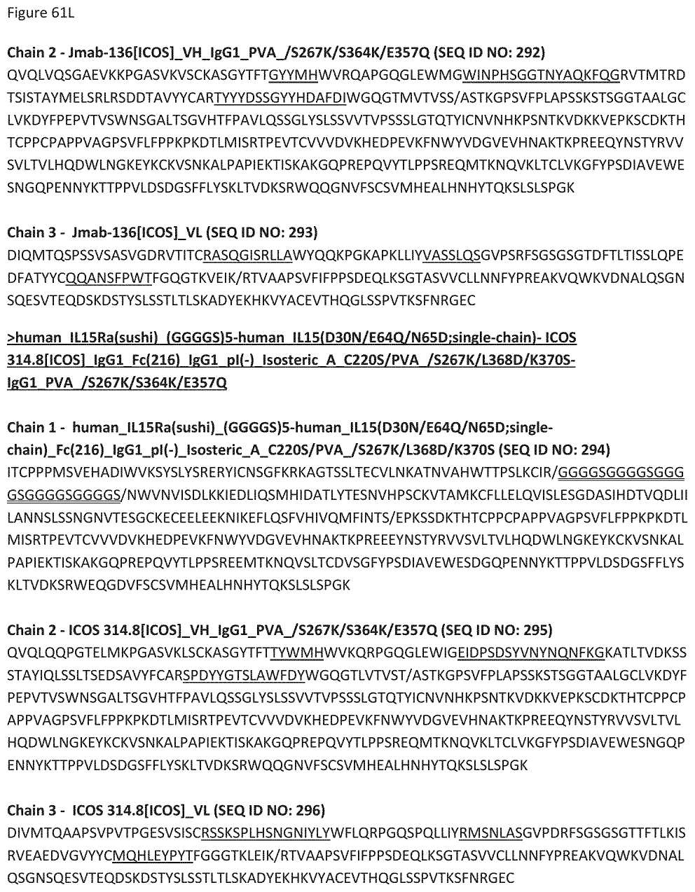

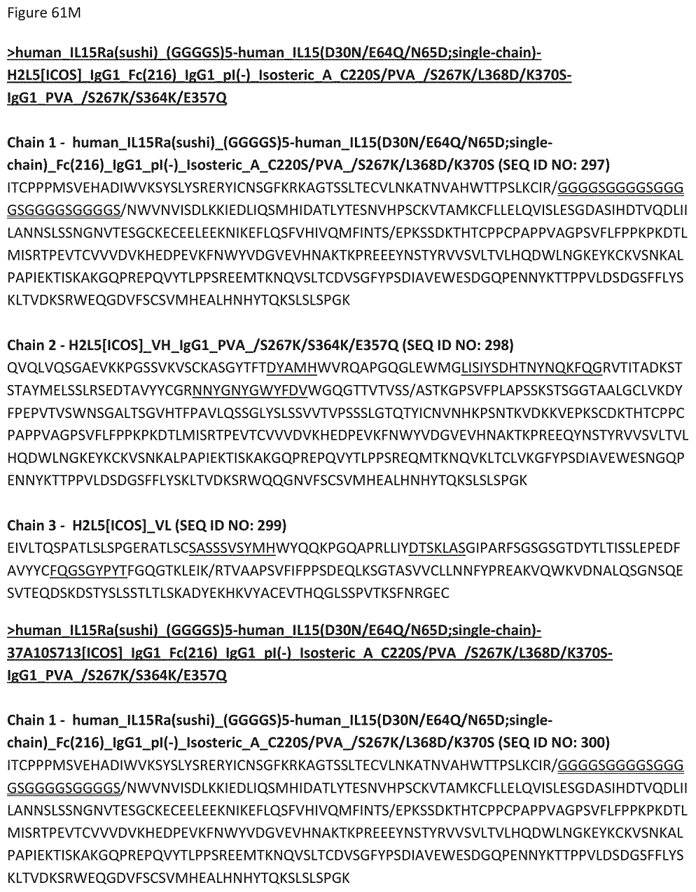

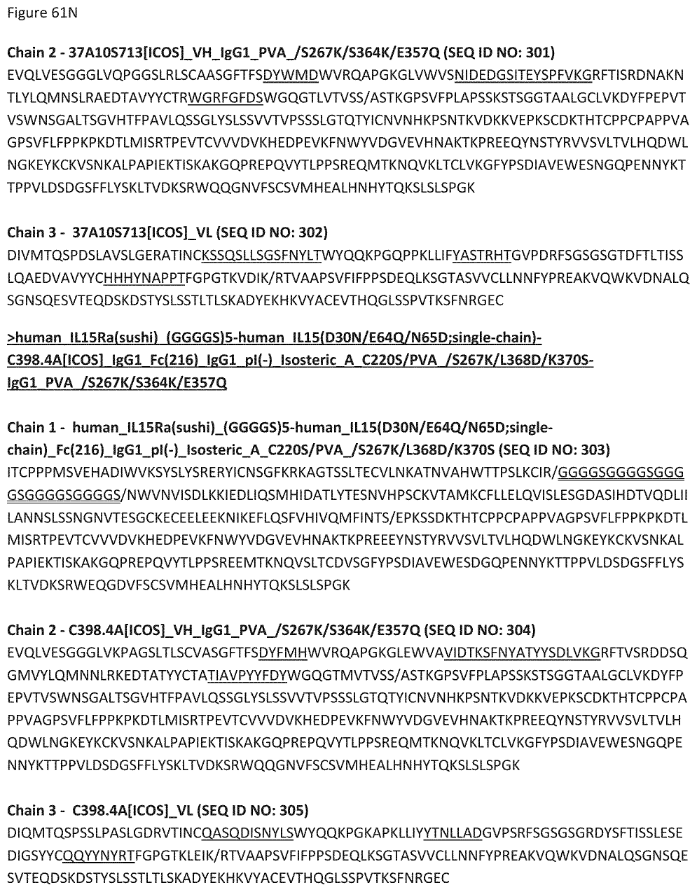

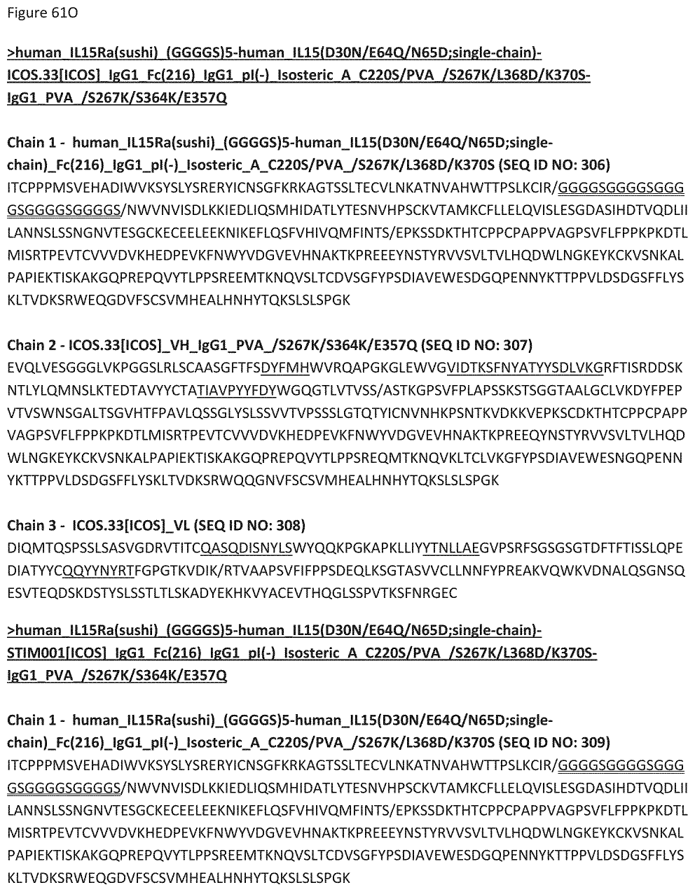

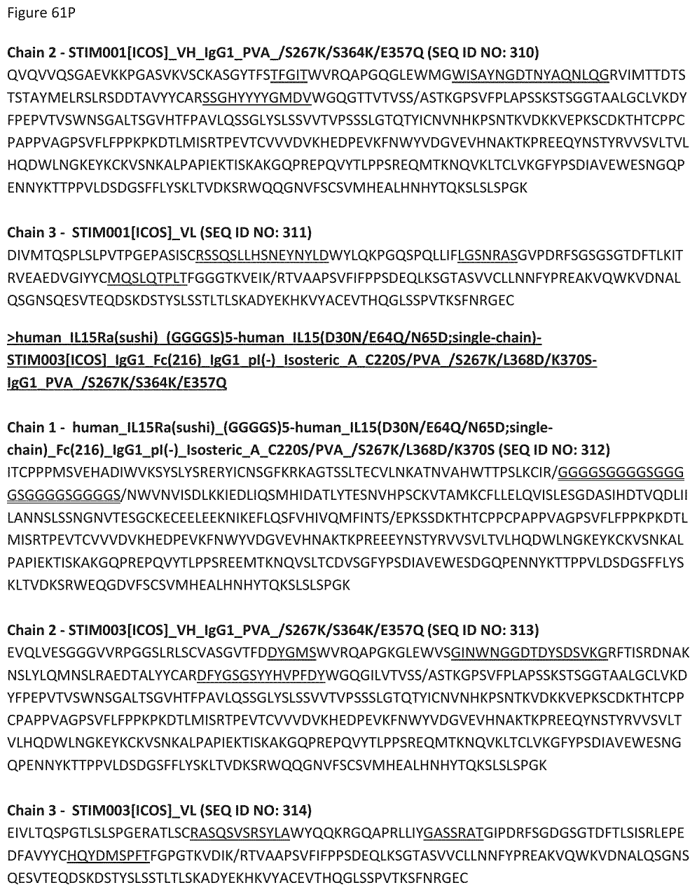

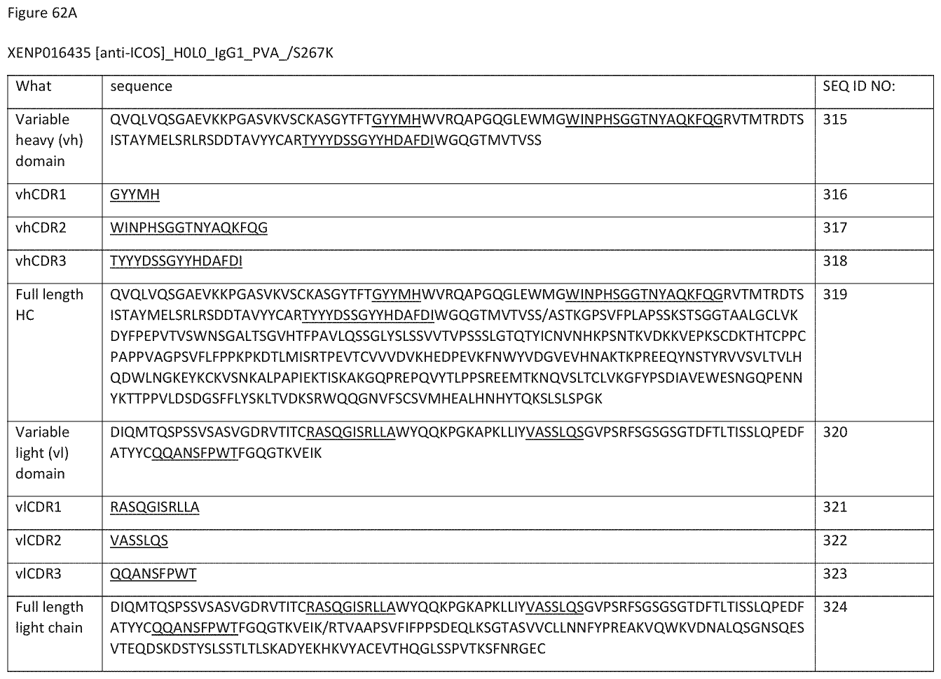

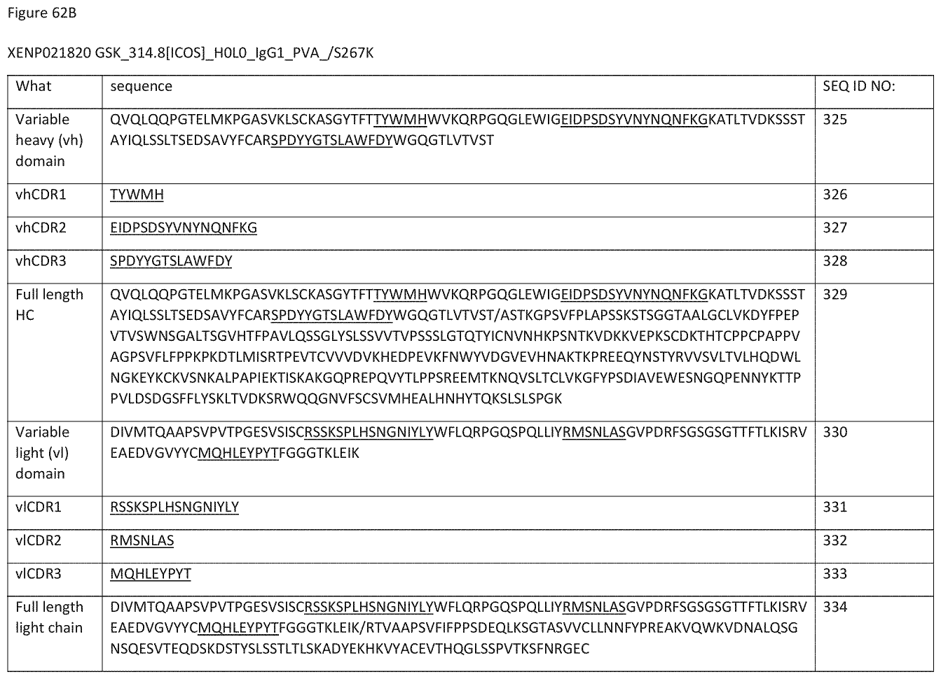

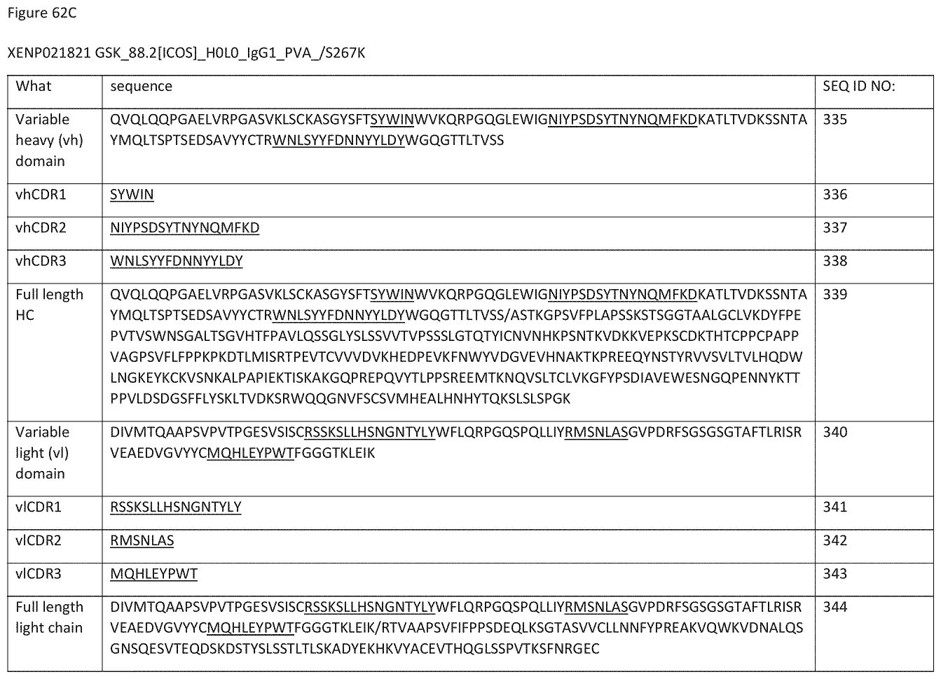

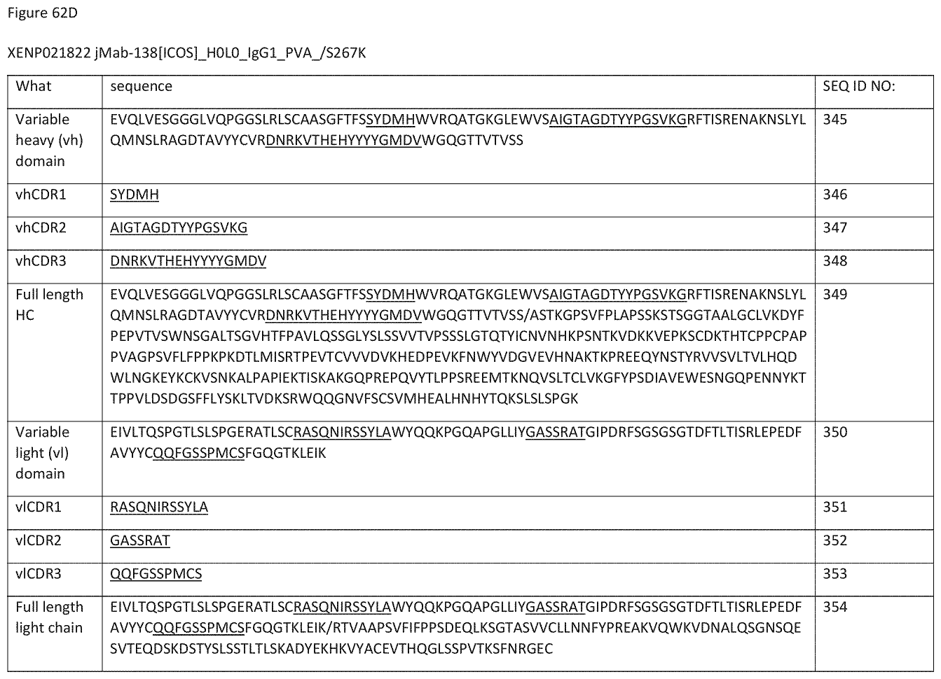

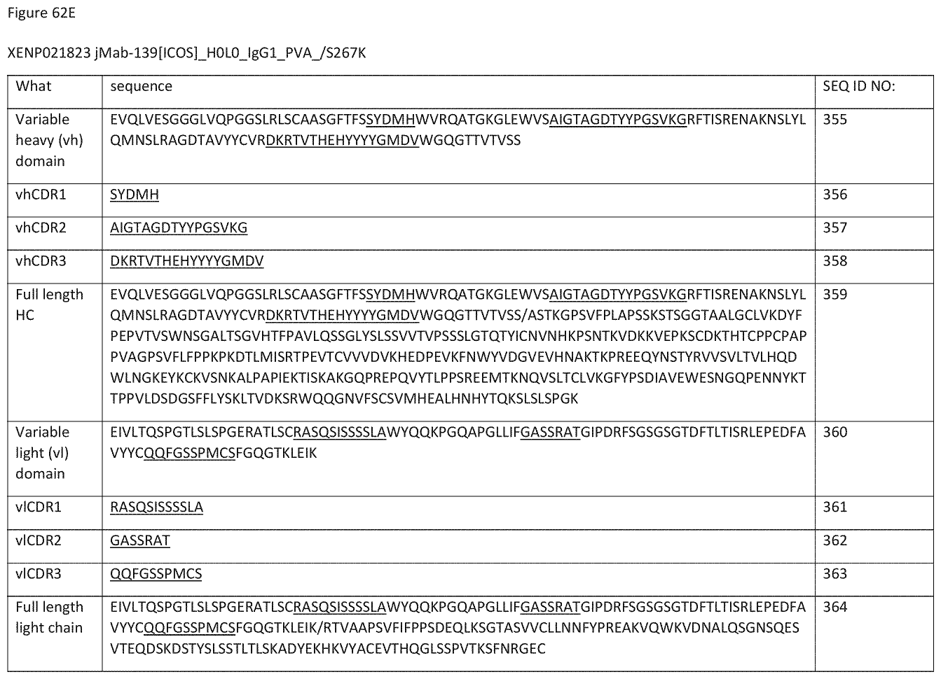

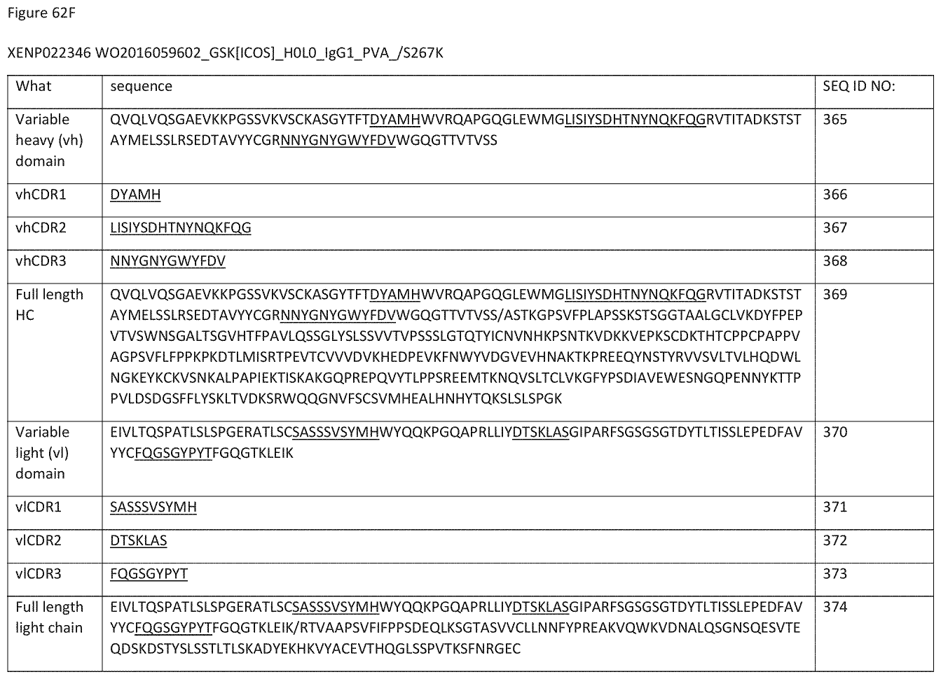

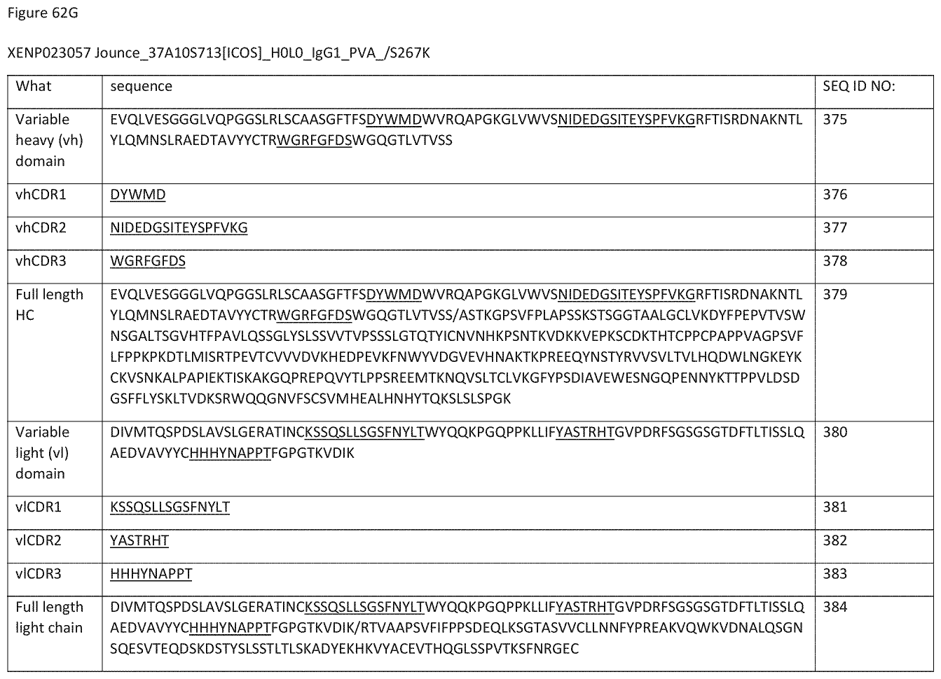

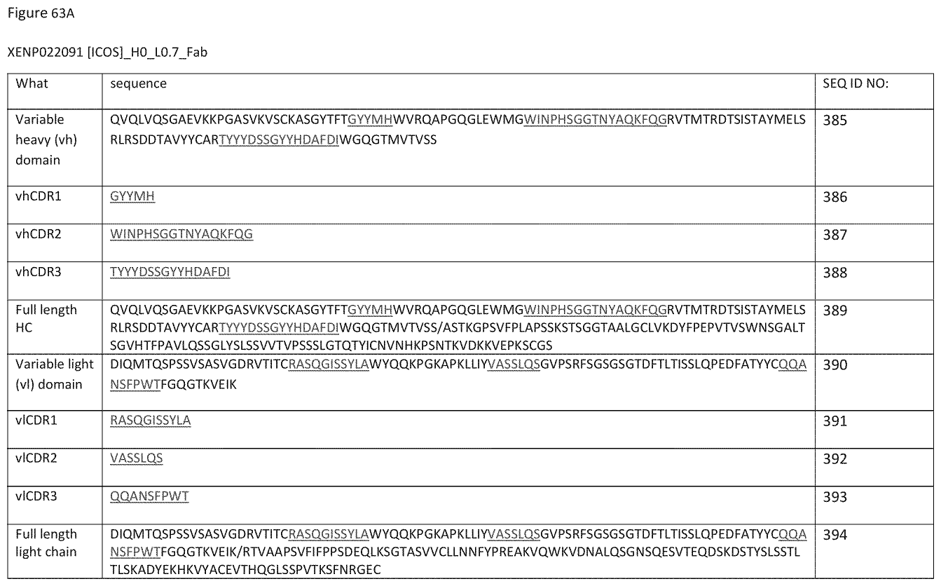

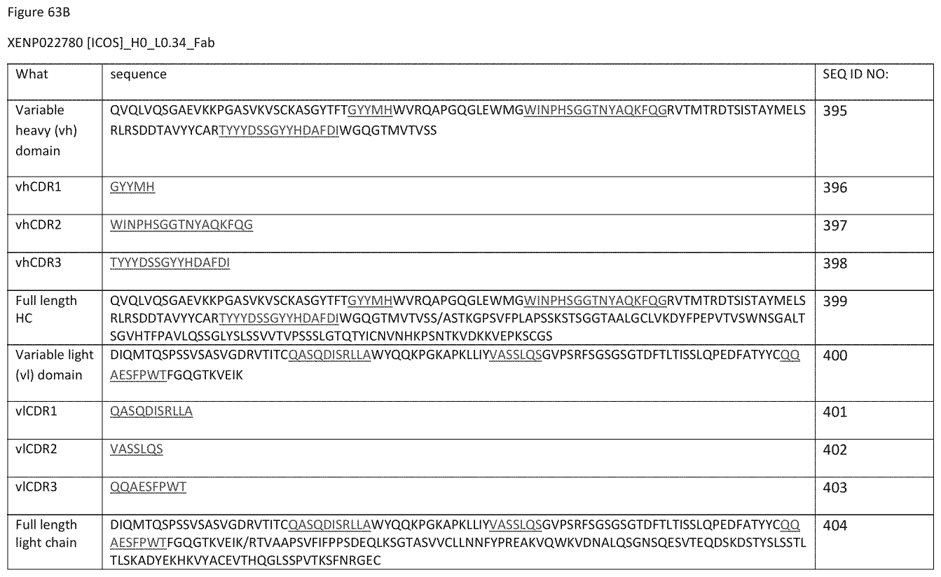

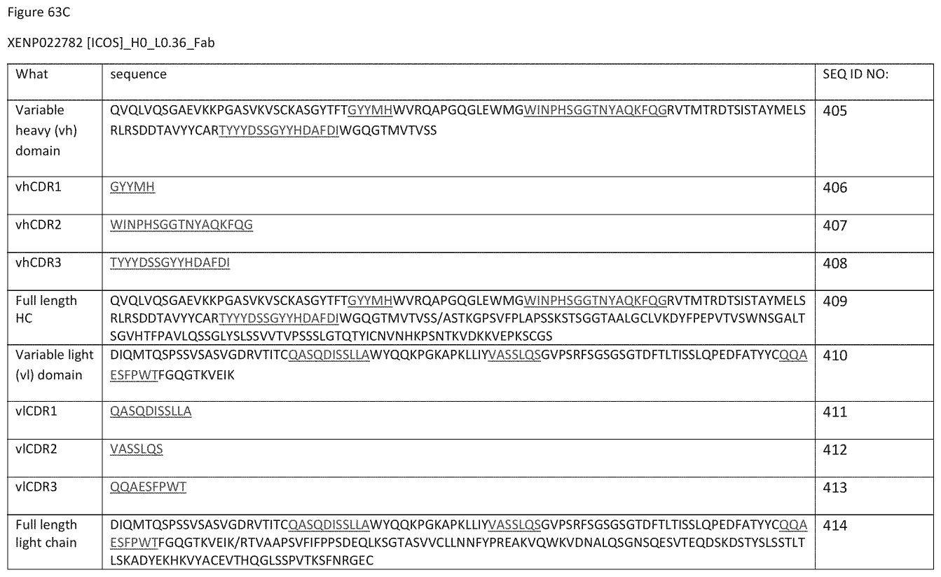

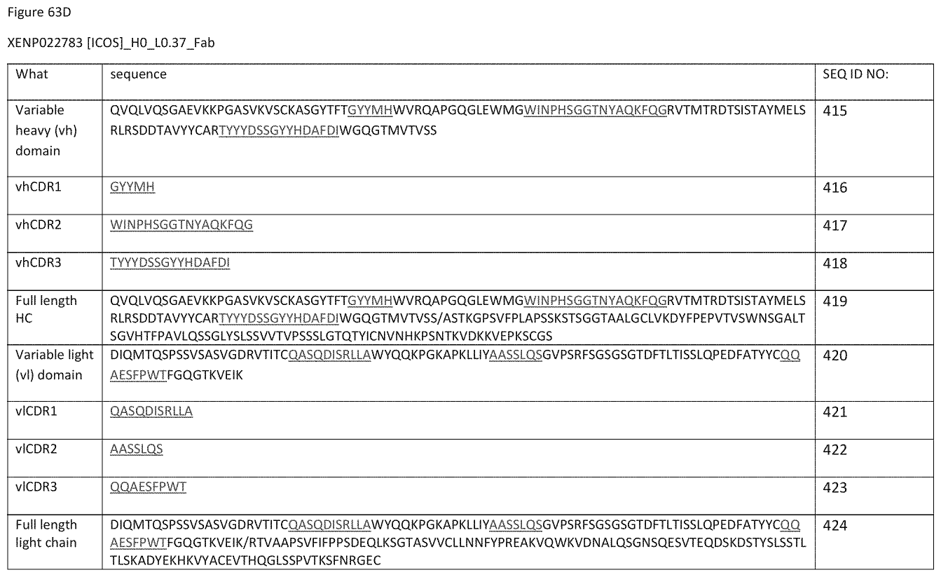

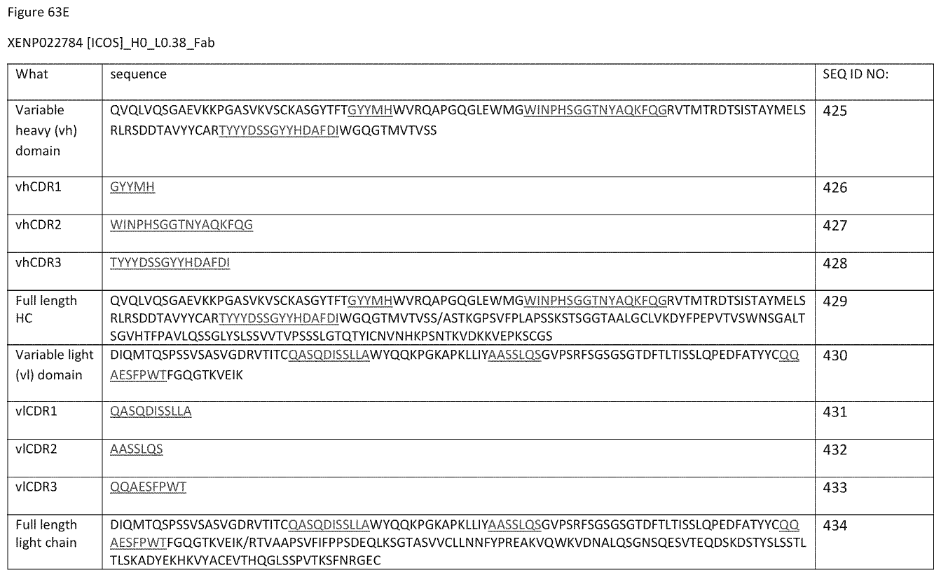

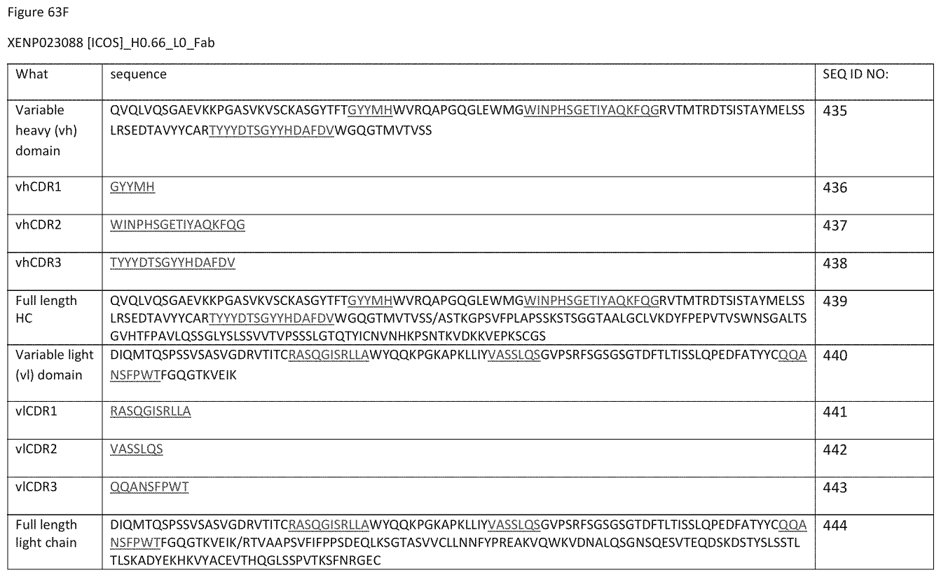

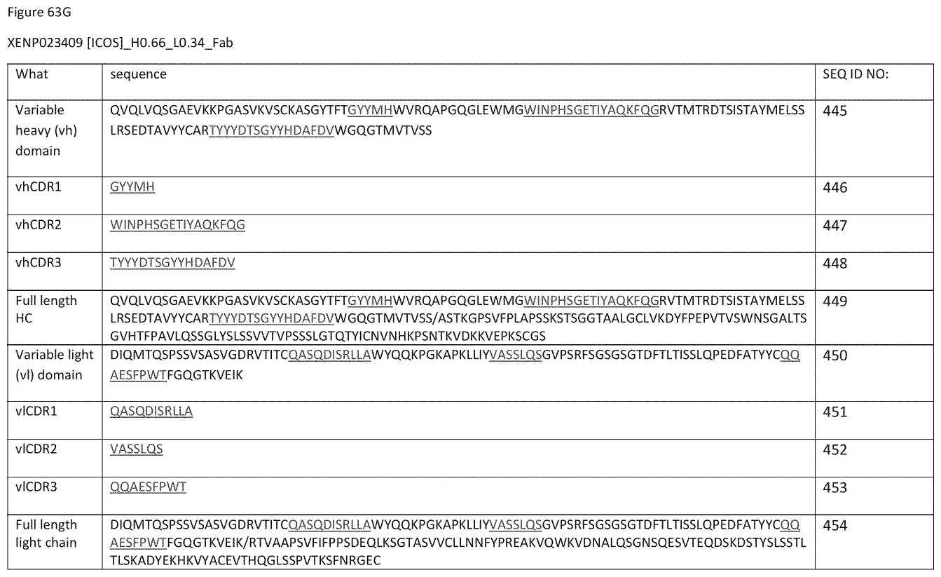

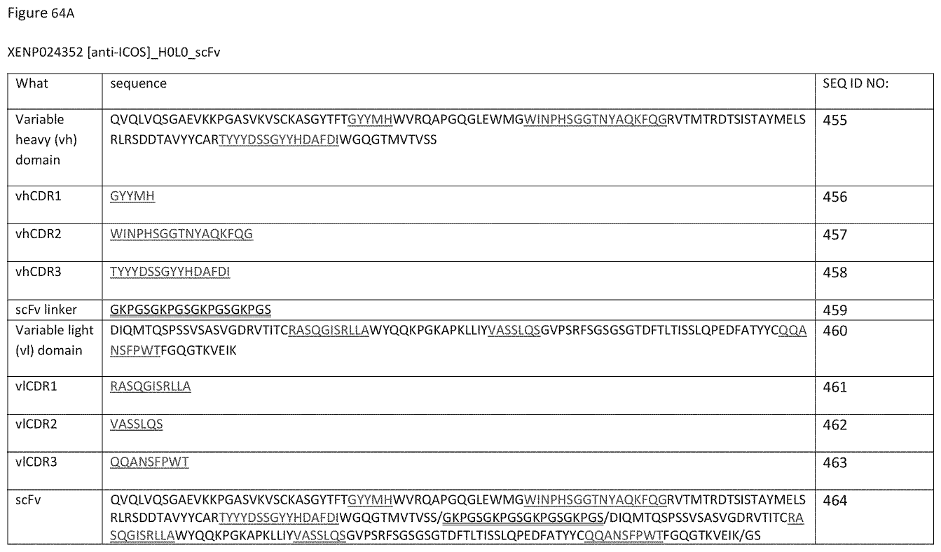

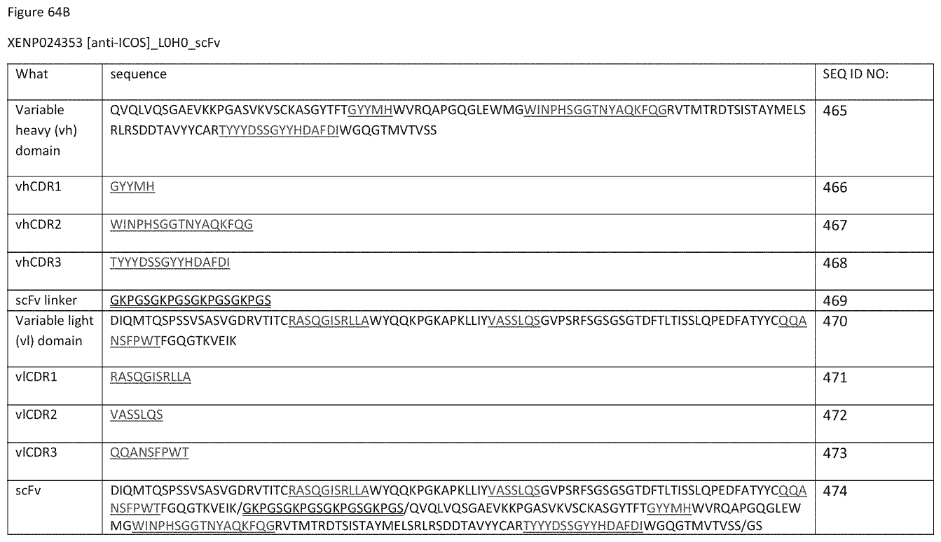

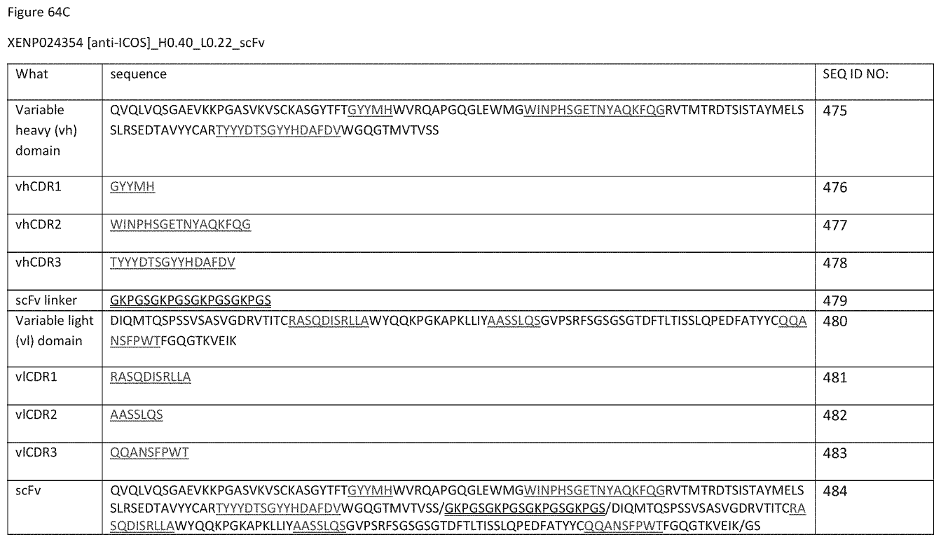

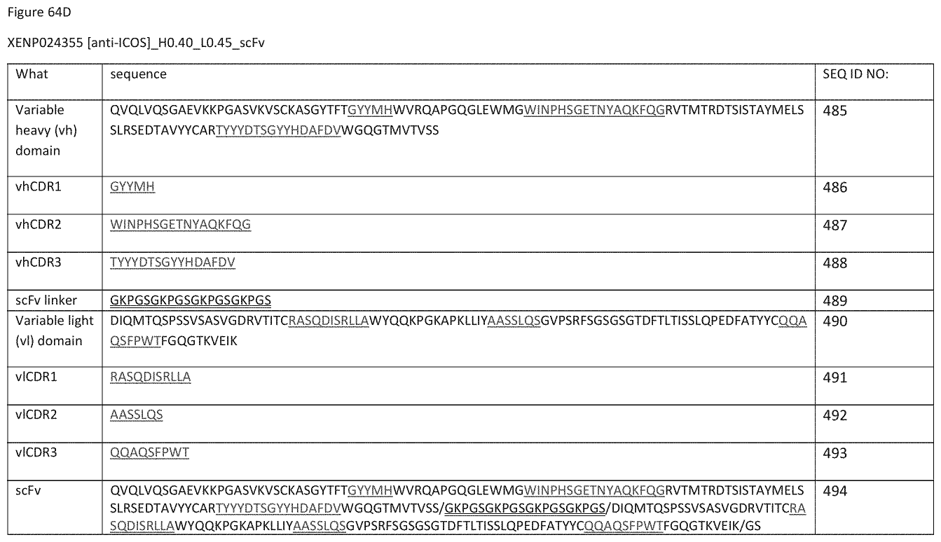

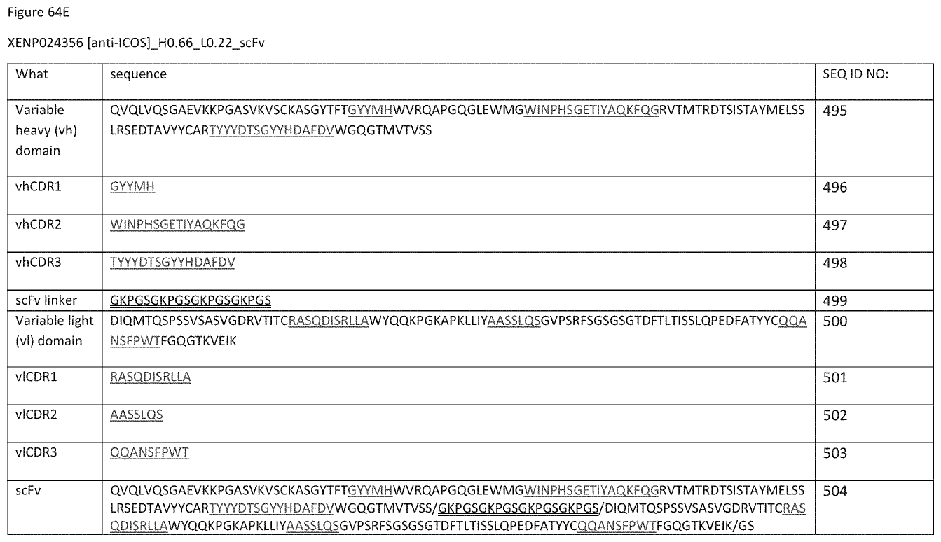

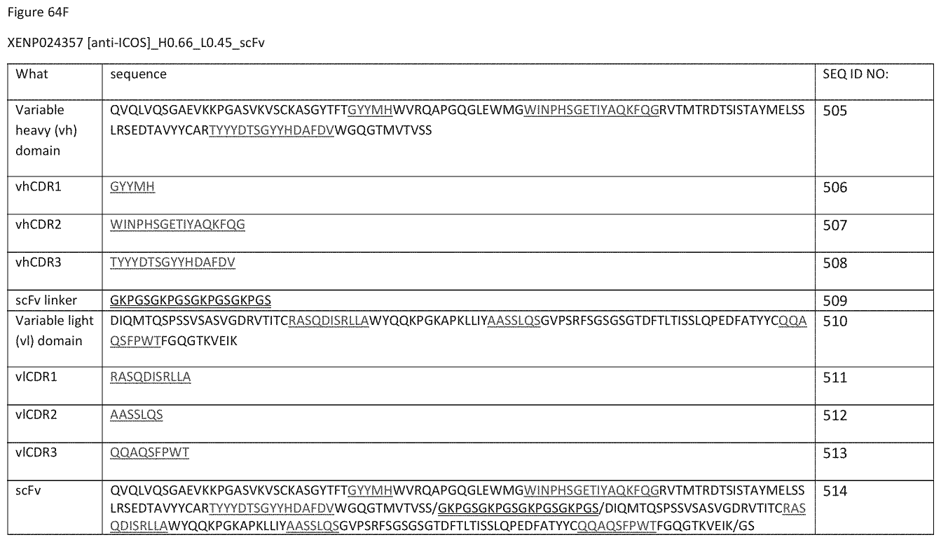

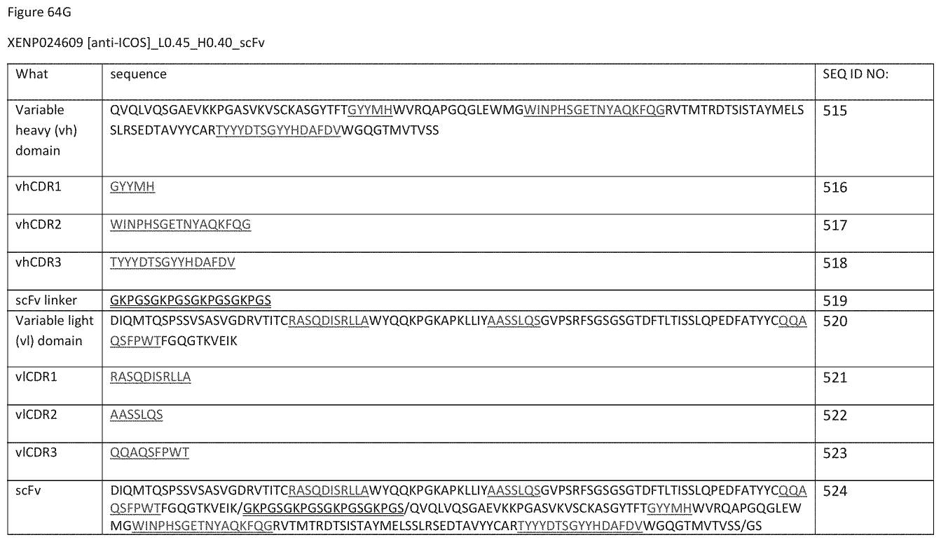

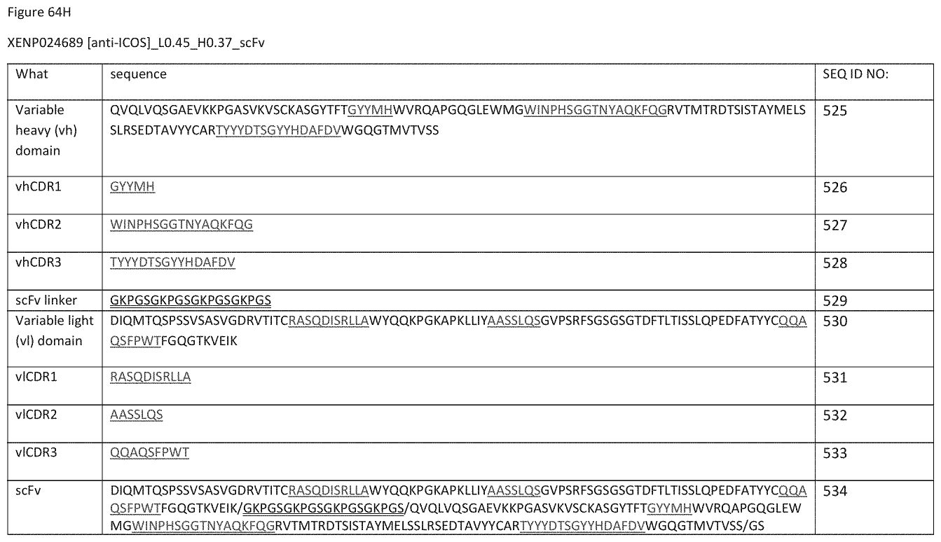

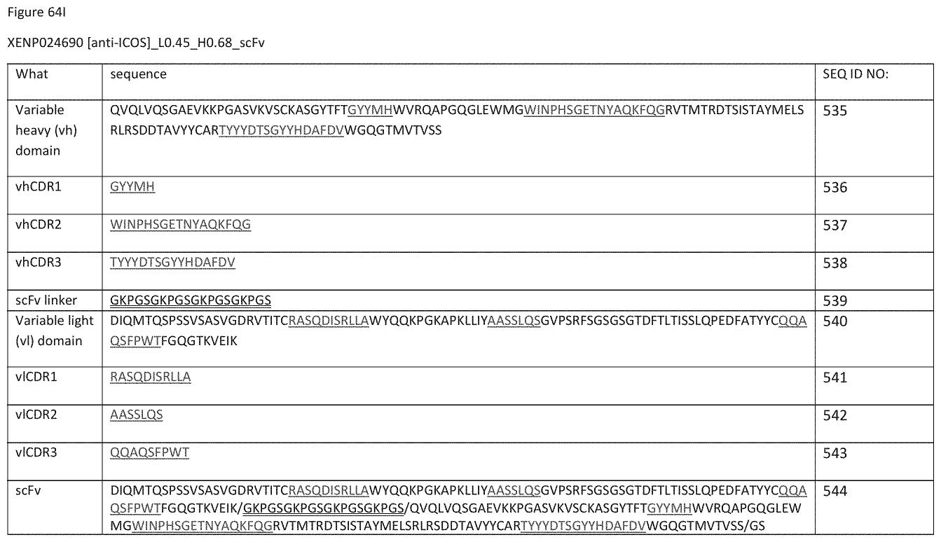

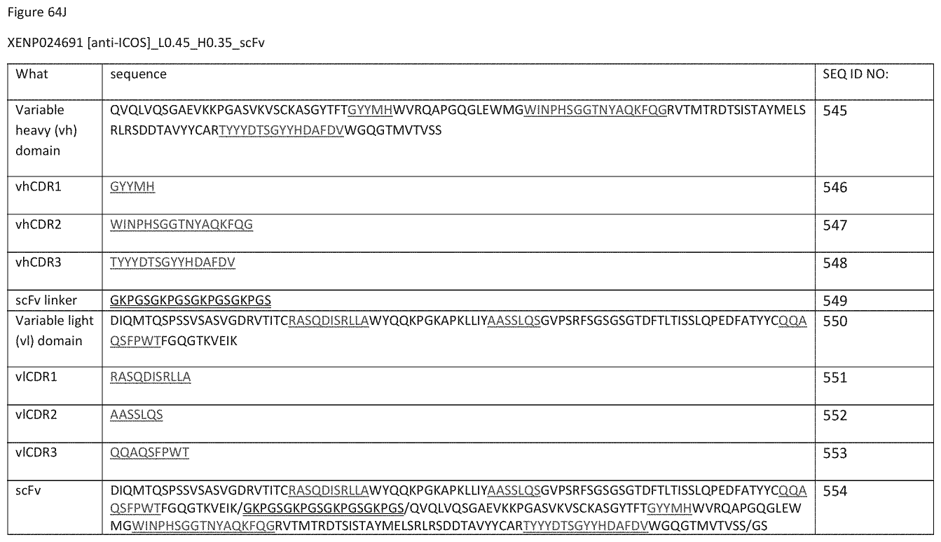

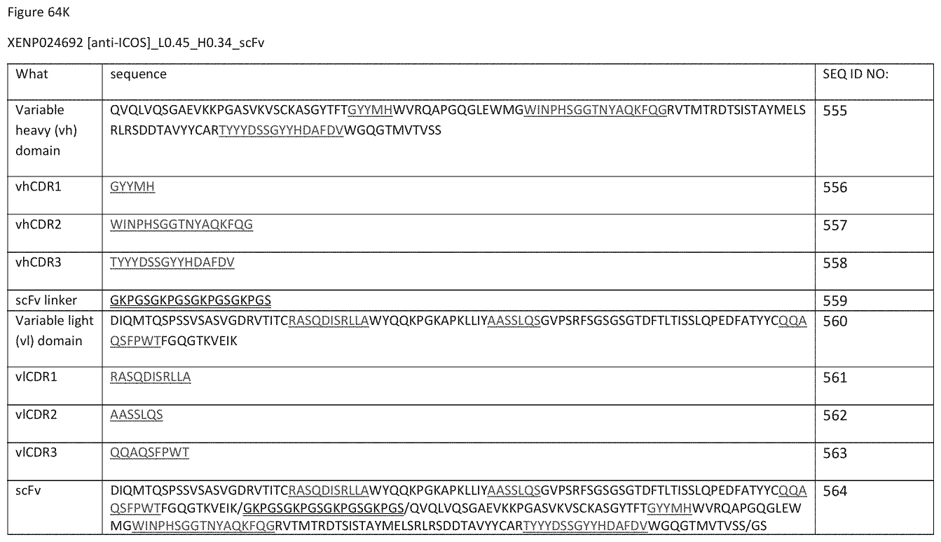

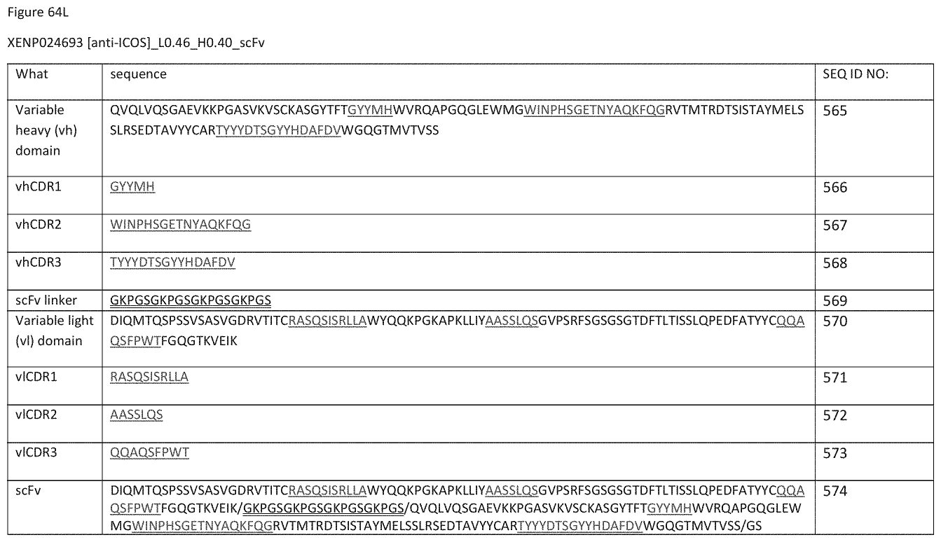

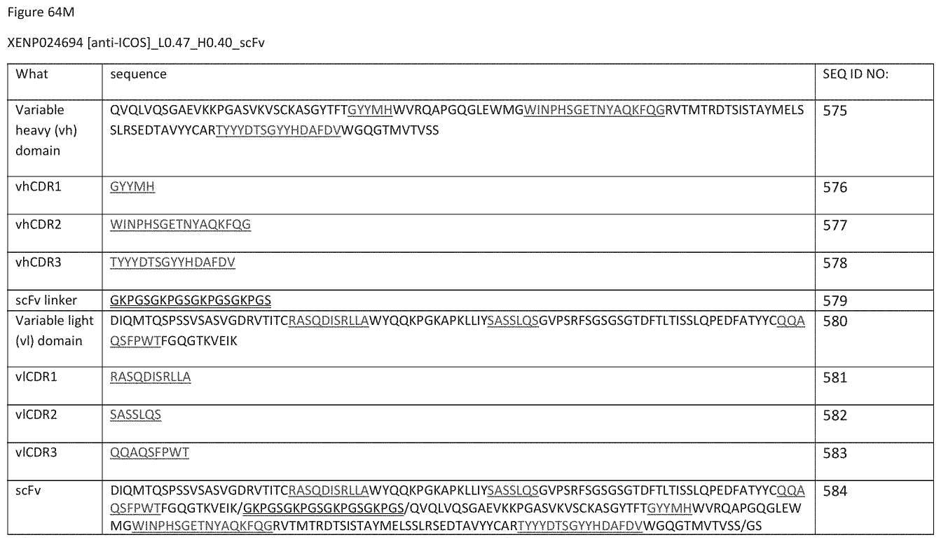

As will be appreciated by those in the art and outlined below, these sequences can be used with any IL-15 and IL-15Rα(sushi) pairs outlined herein, including but not limited to IL-15/Rα-heteroFc, ncIL-15/Rα, and scIL-15/Rα, as schematically depicted in FIG. 14A-FIG. 14G. Additionally, any IL-15 and/or IL-15Rα(sushi) variants can be incorporated into these FIG. 11 backbones in any combination.

Included within each of these backbones are sequences that are 90%, 91%, 92%, 93%, 94%, 95%, 96%, 97%, 98%, and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 12 shows the sequences of several useful ICOS-targeted IL-15/Rα-Fc fusion format backbones based on human IgG1, without the cytokine sequences (e.g. the Il-15 and/or IL-15Rα(sushi)) or VH, and further excluding cognate light chain backbones which are depicted in FIG. 13. Backbone 1 is based on human IgG1 (356E/358M allotype), and includes the S364K/E357Q:L368D/K370S skew variants, C220S and the Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes the S364K/E357Q:L368D/K370S skew variants, the N208D/Q295E/N384D/Q418E/N421D pI variants on the chain with L368D/K370S skew variants, C220S in the chain with S364K/E357Q variants, and the E233P/L234V/L235A/G236del/S267K ablation variants on both chains. Backbone 3 is based on human IgG1 (356E/358M allotype), and includes the S364K/E357Q:L368D/K370S skew variants, the N208D/Q295E/N384D/Q418E/N421D pI variants on the chains with L368D/K370S skew variants, the Q196K/I199T/P217R/P228R/N276K pI variants on the chains with S364K/E357Q variants, and the E233P/L234V/L235A/G236del/S267K ablation variants on both chains.

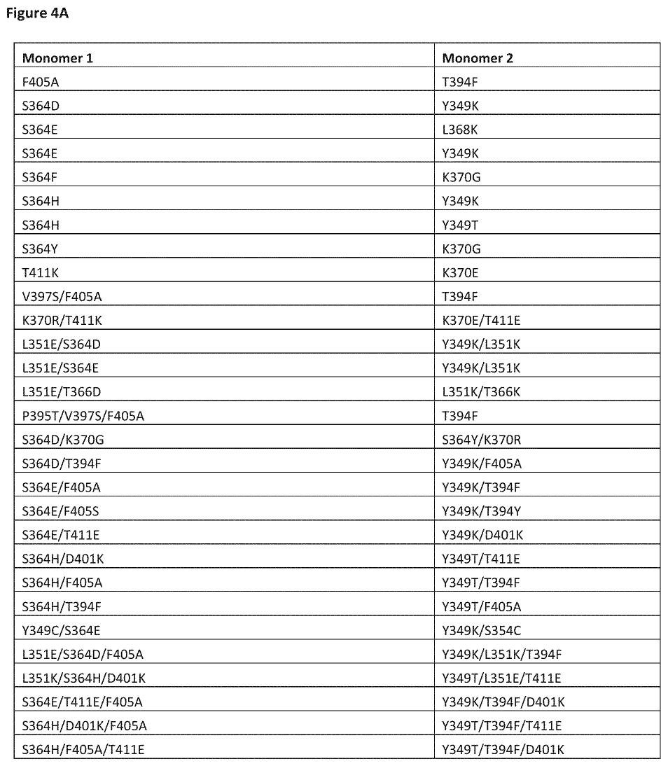

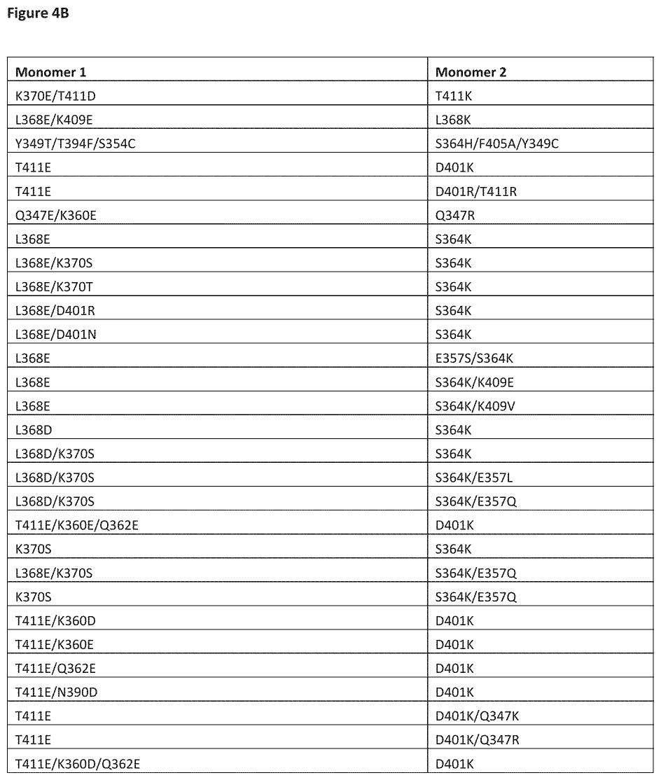

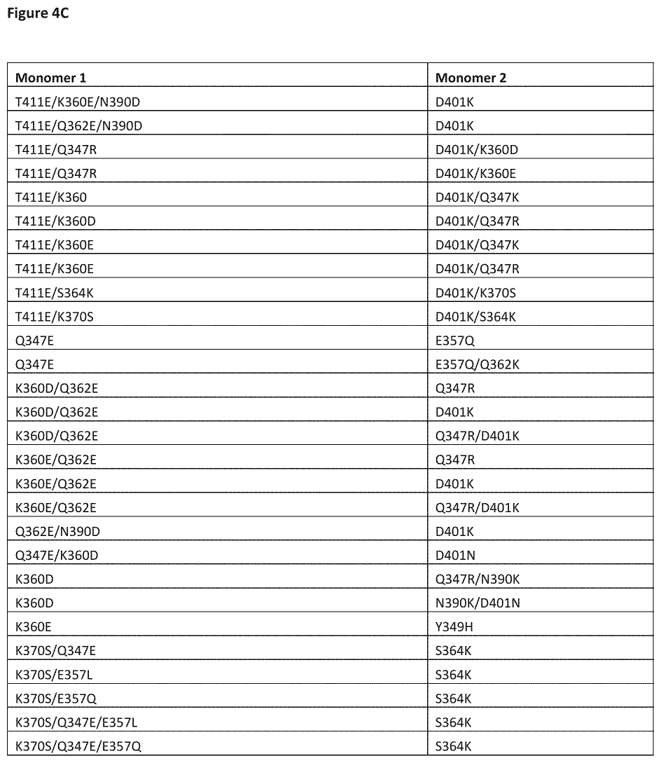

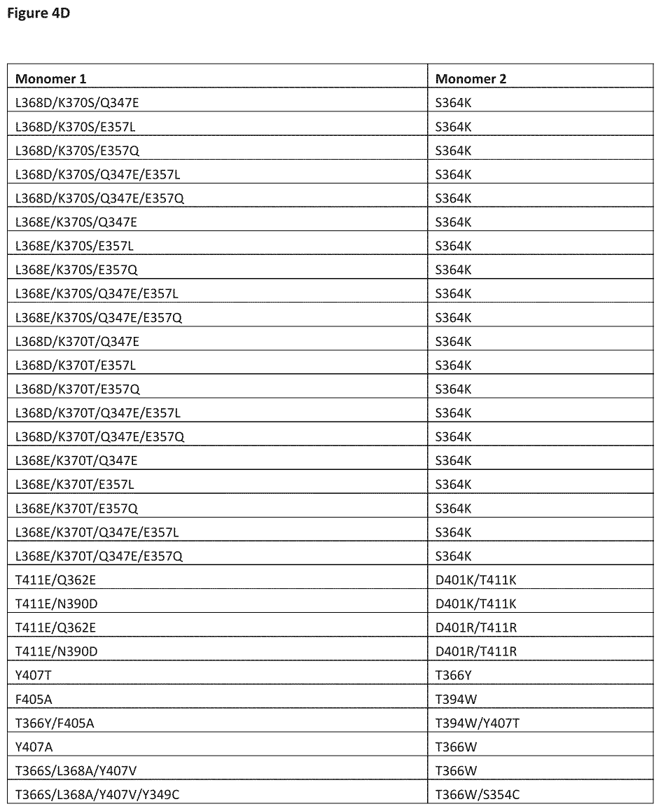

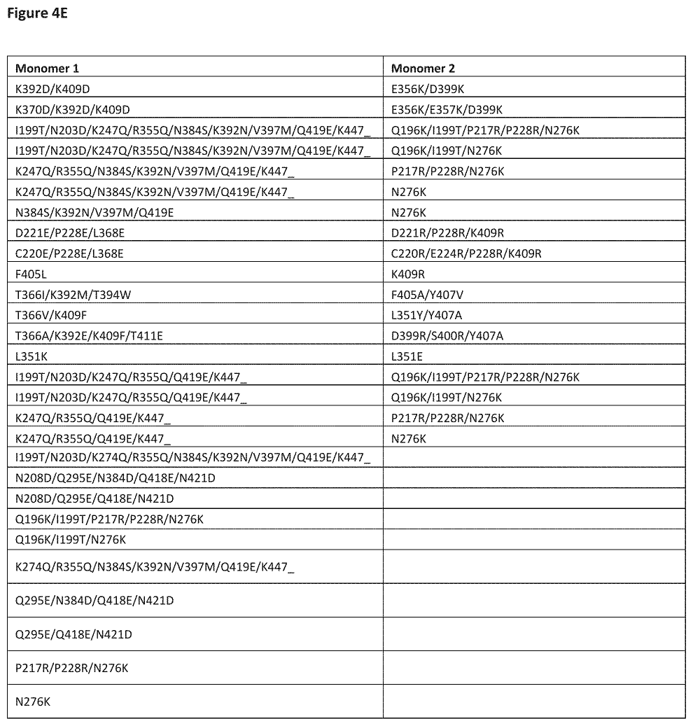

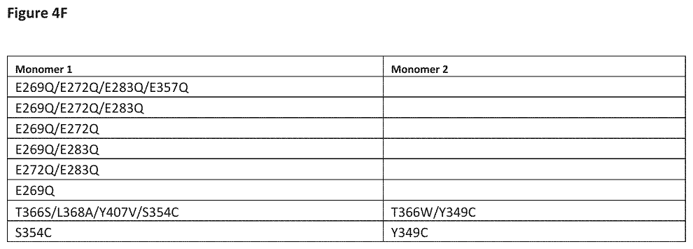

In certain embodiments, these sequences can be of the 356D/358L allotype. In other embodiments, these sequences can include either the N297A or N297S substitutions. In some other embodiments, these sequences can include the M428L/N434S Xtend mutations. In yet other embodiments, these sequences can instead be based on human IgG4, and include a S228P (EU numbering, this is S241P in Kabat) variant on both chains that ablates Fab arm exchange as is known in the art. In yet further embodiments, these sequences can instead be based on human IgG2. Further, these sequences may instead utilize the other skew variants, pI variants, and ablation variants depicted in FIG. 4 to FIG. 6.

As will be appreciated by those in the art and outlined below, these sequences can be used with any IL-15 and IL-15Rα(sushi) pairs outlined herein, including but not limited to scIL-15/Rα, ncIL-15/Rα, and dsIL-15Rα, as schematically depicted in FIG. 32A-FIG. 32H. Further as will be appreciated by those in the art and outlined below, any IL-15 and/or IL-15Rα(sushi) variants can be incorporated in these backbones. Furthermore as will be appreciated by those in the art and outlined below, these sequences can be used with any VH and VL pairs outlined herein, including either a scFv or a Fab.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 13 depicts the “non-Fv” backbone of cognate light chains (i.e. constant light chain) which find use in ICOS-targeted IL-15/Rα-Fc fusion proteins of the invention.

FIGS. 14A-14G depict several formats for the IL-15/Rα-Fc fusion proteins of the present invention. IL-15Rα Heterodimeric Fc fusion or “IL-15/Rα-heteroFc” (FIG. 14A) comprises IL-15 (including an IL-15 variant) recombinantly fused to one side of a heterodimeric Fc and IL-15Rα(sushi) recombinantly fused to the other side of a heterodimeric Fc. The IL-15 and IL-15Rα(sushi) may have a variable length Gly-Ser linker between the C-terminus and the N-terminus of the Fc region. Single-chain IL-15/Rα-Fc fusion or “scIL-15/Rα-Fc” (FIG. 14B) comprises IL-15Rα(sushi) fused to IL-15 by a variable length linker (termed a “single-chain” IL-15/IL-15Rα(sushi) complex or “scIL-15/Rα”) which is then fused to the N-terminus of a heterodimeric Fc-region, with the other side of the molecule being “Fc-only” or “empty Fc”. Non-covalent IL-15/Rα-Fc or “ncIL-15/Rα-Fc” (FIG. 14C) comprises IL-15Rα(sushi) fused to a heterodimeric Fc region, while IL-15 is transfected separately so that a non-covalent IL-15/Rα complex is formed, with the other side of the molecule being “Fc-only” or “empty Fc”. Bivalent non-covalent IL-15/Rα-Fc fusion or “bivalent ncIL-15/Rα-Fc” (FIG. 14D) comprises IL-15Rα(sushi) fused to the N-terminus of a homodimeric Fc region, while IL-15 is transfected separately so that a non-covalent IL-15/Rα complex is formed. Bivalent single-chain IL-15/Rα-Fc fusion or “bivalent scIL-15/Rα-Fc” (FIG. 14E) comprises IL-15 fused to IL-15Rα(sushi) by a variable length linker (termed a “single-chain” IL-15/IL-15Rα(sushi) complex or “scIL-15/Rα”) which is then fused to the N-terminus of a homodimeric Fc-region. Fc-non-covalent IL-15/Rα fusion or “Fc-ncIL-15/Rα” (FIG. 14F) comprises IL-15Rα(sushi) fused to the C-terminus of a heterodimeric Fc region, while IL-15 is transfected separately so that a non-covalent IL-15/Rα complex is formed, with the other side of the molecule being “Fc-only” or “empty Fc”. Fc-single-chain IL-15/Rα fusion or “Fc-scIL-15/Rα” (FIG. 14G) comprises IL-15 fused to IL-15Rα(sushi) by a variable length linker (termed a “single-chain” IL-15/IL-15Rα(sushi) complex or “scIL-15/Rα”) which is then fused to the C-terminus of a heterodimeric Fc region, with the other side of the molecule being “Fc-only” or “empty Fc”.

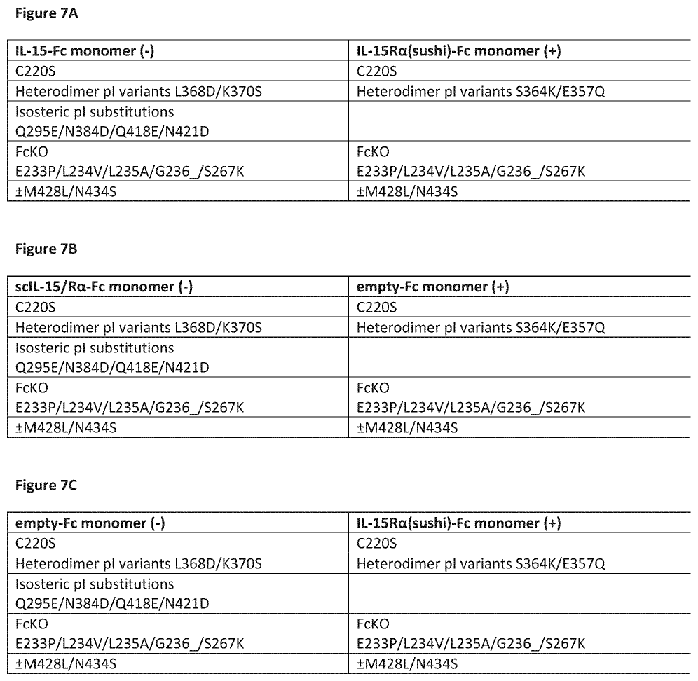

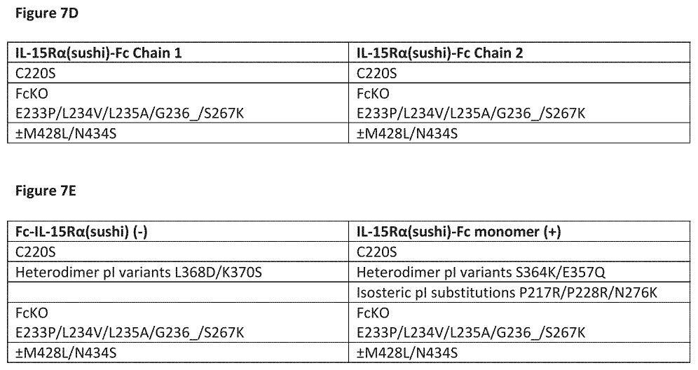

FIG. 15 depicts sequences of illustrative IL-15/Rα-Fc fusion proteins of the “IL-15/Rα-heteroFc” format. IL-15 and IL-15Rα(sushi) are underlined, linkers are double underlined (although as will be appreciated by those in the art, the linkers can be replaced by other linkers, some of which are depicted in FIG. 7), and slashes (/) indicate the border(s) between IL-15, IL-15Rα, linkers, and Fc regions.

FIG. 16 depicts sequences of illustrative IL-15/Rα-Fc fusion proteins of the “scIL-15/Rα-Fc” format. IL-15 and IL-15Rα(sushi) are underlined, linkers are double underlined (although as will be appreciated by those in the art, the linkers can be replaced by other linkers, some of which are depicted in FIG. 7), and slashes (/) indicate the border(s) between IL-15, IL-15Rα, linkers, and Fc regions.

FIG. 17 depicts sequences of illustrative IL-15/Rα-Fc fusion proteins of the “ncIL-15/Rα-Fc” format. IL-15 and IL-15Rα(sushi) are underlined, linkers are double underlined (although as will be appreciated by those in the art, the linkers can be replaced by other linkers, some of which are depicted in FIG. 7), and slashes (/) indicate the border(s) between IL-15, IL-15Rα, linkers, and Fc regions.

FIGS. 18A-18C depict the induction of A) NK (CD56⁺/CD16⁺) cells, B) CD4⁺ T cells, and C) CD8⁺ T cells proliferation by illustrative IL-15/Rα-Fc fusion proteins of scIL-15/Rα-Fc format (XENP21478) and ncIL-15/Rα-Fc format (XENP21479) based on Ki67 expression as measured by FACS.

FIG. 19 depicts the structure of IL-15 complexed with IL-15Rα, IL-2Rβ, and common gamma chain. Locations of substitutions designed to reduce potency are shown.

FIGS. 20A-20D depict sequences for illustrative IL-15 variants engineered with the aim to reduce potency. Included within each of these variant IL-15 sequences are sequences that are 90, 95, 98 and 99% identical (as defined herein) to the recited sequences, and/or contain from 1, 2, 3, 4, 5, 6, 7, 8, 9 or 10 additional amino acid substitutions. As will be clear to those skilled in the art, the IL-15 variants can be used in any of the IL-15/Rα-Fc fusion and ICOS-targeted IL-15/Rα-Fc fusion proteins described herein.

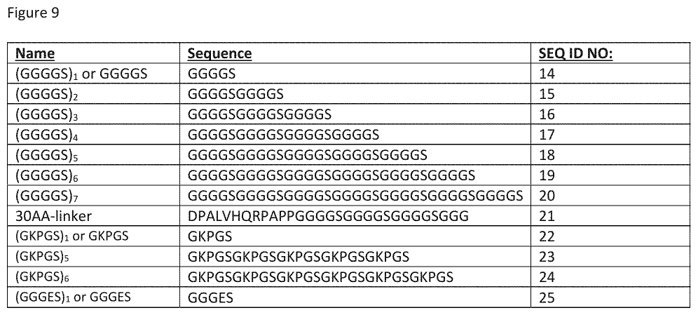

FIGS. 21A-21B depicts sequences of illustrative IL-15/Rα-Fc fusion proteins of the “scIL-15/Rα-Fc” format comprising IL-15 variants engineered with the aim to reduce potency. IL-15 and IL-15Rα(sushi) are underlined, linkers are double underlined (although as will be appreciated by those in the art, the linkers can be replaced by other linkers, some of which are depicted in FIG. 9), and slashes (/) indicate the border(s) between IL-15, IL-15Rα, linkers, and Fc regions.

FIGS. 22A-22G depict percentage of A) CD4+CD45RA−, B) CD4+CD45RA+, C) CD8+CD45RA−, D) CD8+CD45RA+, E) CD16+NK cells, F) CD56+NK cells, and G) γδ cells expression Ki67 following incubation with the indicated test articles.

FIGS. 23A-23B depict A) natural transpresentation of IL-15:IL-15Rα complex and costimulation ligand (e.g. ICOS-L) to T cells, and B) the analogous presentation of IL-15:IL-15Rα and costimulation by the ICOS-targeted IL-15/Rα-Fc fusion proteins of the invention.

FIG. 24 depicts the variable heavy and variable light chains for illustrative ICOS antigen binding domains (ABD. The variable heavy chains, variable light chains, and six CDRs of such ABDs find use in the fusion proteins provided herein. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems.

FIGS. 25A-25CC depict several formats for the ICOS-targeted IL-15/Rα-Fc fusion proteins of the present invention. The “scIL15Rα-IL15-Fc x scFv-Fc” format (FIG. 25A) comprises two monomers—the first monomer comprises, from N- to C-terminus, the IL-15Rα(sushi) domain-(domain linker)-IL-15 variant-(domain linker)-CH2-CH3 (with the second domain linker frequently being a hinge domain); and the second monomer comprises VH-scFv linker-VL-hinge-CH2-CH3 or VL-scFv linker-VH-hinge-CH2-CH3, although in either orientation a domain linker can be substituted for the hinge. The “scIL15-IL15Rα-Fc x scFv-Fc” format (FIG. 25B) comprises two monomers—the first monomer comprises, from N- to C-terminus, the IL-15 variant-(domain linker)-IL-15Rα(sushi) domain-(domain linker)-CH2-CH3 (with the second domain linker frequently being a hinge domain); and the second monomer comprises VH-scFv linker-VL-hinge-CH2-CH3 or VL-scFv linker-VH-hinge-CH2-CH3, although in either orientation a domain linker can be substituted for the hinge. The “IL15-Fc x scFv-Fc” format (FIG. 25C) comprises two monomers—the first monomer comprises, from N- to C-terminus, the IL-15 variant-(domain linker)-CH2-CH3 (with the second domain linker frequently being a hinge domain), and the second monomer comprises VH-scFv linker-VL-hinge-CH2-CH3 or VL-scFv linker-VH-hinge-CH2-CH3, although in either orientation a domain linker can be substituted for the hinge. The “ncIL15+IL15Rα-Fc x scFv-Fc” format (FIG. 25D) comprises three monomers—the first monomer comprises, from N- to C-terminus, the IL-15Rα(sushi) domain-domain linker-CH2-CH3; the second monomer comprises vh-scFv linker-vl-hinge-CH2-CH3 or vl-scFv linker-vh-hinge-CH2-CH3, although in either orientation a domain linker can be substituted for the hinge; the third monomer is the variant IL-15 domain that self-assembles with the IL-15Rα(sushi) domain. The “ncIL15Rα+IL15-Fc x scFv-Fc” format (FIG. 25E) comprises three monomers—the first monomer comprises, from N- to C-terminus, a variant IL15-domain linker-CH2-CH3; the second monomer comprises vh-scFv linker-vl-hinge-CH2-CH3 or vl-scFv linker-vh-hinge-CH2-CH3, although in either orientation a domain linker can be substituted for the hinge; and the third monomer is the IL-15Rα(sushi) domain that self-assembles with the IL-15. The “dsIL15+IL15Rα-Fc x scFv-Fc” format (FIG. 25F) comprises three monomers—the first monomer comprises, from N- to C-terminus, the a variant IL-15Rα(sushi) domain-domain linker-CH2-CH3, wherein the variant IL-15Rα(sushi) domain has an engineered cysteine residue; the second monomer comprises vh-scFv linker-vl-hinge-CH2-CH3 or vl-scFv linker-vh-hinge-CH2-CH3, although in either orientation a domain linker can be substituted for the hinge; and the third monomer is the variant IL-15 domain, also engineered to have a cysteine variant amino acid, thus allowing a disulfide bridge to form between the IL-15Rα(sushi) domain and the variant IL-15 domain. The “dsIL15Rα+IL15-Fc x scFv-Fc” format (FIG. 25G) comprises three monomers—the first monomer comprises, from N- to C-terminus, a variant IL-15-domain linker-CH2-CH3, wherein the variant IL-15 has an engineered cysteine residue; the second monomer comprises vh-scFv linker-vl-hinge-CH2-CH3 or vl-scFv linker-vh-hinge-CH2-CH3, although in either orientation a domain linker can be substituted for the hinge; and the third monomer is a variant IL-15Rα(sushi) domain, also engineered to have a cysteine variant amino acid, thus allowing a disulfide bridge to form between the IL-15Rα(sushi) domain and the variant IL-15. The “scIL15Rα-IL15-Fc x Fab-Fc” format (FIG. 25H) comprises three monomers—the first monomer comprises, from N- to C-terminus, the IL-15Rα(sushi) domain-domain linker-variant IL-15-domain linker-CH2-CH3; the second monomer comprises a heavy chain, VH-CH1-hinge-CH2-CH3; and the third monomer is a light chain, VL-CL. The “scIL15-IL15Rα-Fc x Fab-Fc” format (FIG. 25I) comprises three monomers—the first monomer comprises, from N- to C-terminus, a variant IL-15-domain linker-IL-15Rα(sushi) domain-domain linker-CH2-CH3; the second monomer comprises a heavy chain, VH-CH1-hinge-CH2-CH3; and the third monomer is a light chain, VL-CL. The “IL15-Fc x Fab-Fc” format (FIG. 25J) comprises three monomers—the first monomer comprises, from N- to C-terminus, a variant IL-15-domain linker-CH2-CH3; the second monomer comprises a heavy chain, VH-CH1-hinge-CH2-CH3; and the third monomer is a light chain, VL-CL. The “ncIL15+IL15Rα-Fc x Fab-Fc” format (FIG. 25K) comprises three monomers—the first monomer comprises, from N- to C-terminus, the IL-15Rα(sushi) domain-domain linker-CH2-CH3; the second monomer comprises a heavy chain, VH-CH1-hinge-CH2-CH3; and the third monomer is the variant IL-15 domain that self-assembles with the IL-15. The “ncIL15Rα+IL15-Fc x Fab-Fc” format (FIG. 25L) comprises three monomers—the first monomer comprises, from N- to C-terminus, the variant IL-15-domain linker-CH2-CH3; the second monomer comprises a heavy chain, VH-CH1-hinge-CH2-CH3; and the third monomer is the IL-15Rα(sushi) domain that self-assembles with the IL-15. The “dsIL15+IL15Rα-Fc x Fab-Fc” format (FIG. 25M) comprises three monomers—the first monomer comprises, from N- to C-terminus, the a variant IL-15Rα(sushi)domain-domain linker-CH2-CH3, wherein the variant IL-15Rα(sushi)domain has been engineered to contain a cysteine residue; the second monomer comprises a heavy chain, VH-CH1-hinge-CH2-CH3; and the third monomer is the variant IL-15 domain, also engineered to have a cysteine residue, such that a disulfide bridge is formed under physiological or native cellular conditions. The “dsIL15Rα+IL15-Fc x Fab-Fc” format (FIG. 25N) comprises three monomers—the first monomer comprises, from N- to C-terminus, a variant IL-15-domain linker-CH2-CH3, wherein the variant IL-15 has been engineered to contain a cysteine residue; the second monomer comprises a heavy chain, VH-CH1-hinge-CH2-CH3; and the third monomer is the variant IL-15Rα(sushi) domain, also engineered to have a cysteine residue, such that a disulfide bridge is formed under physiological or native cellular conditions. The “Fab-Fc-scIL15Rα-IL15 x Fab-Fc” format (FIG. 25O) comprises three monomers (although the fusion protein is a tetramer)—the first monomer comprises a heavy chain, VH-CH1-hinge-CH2-CH3; the second monomer comprises a heavy chain with a C-terminal scIL15Rα-IL15 scIL-15 complex e.g. VH-CH1-hinge-CH2-CH3-domain linker-IL-15Rα(sushi)domain-domain linker-IL-15 variant; and the third (and fourth) monomer are light chains, VL-CL. The “Fab-Fc-scIL15-IL15Rα x Fab-Fc” format (FIG. 25P) comprises three monomers (although the fusion protein is a tetramer)—the first monomer comprises a heavy chain, VH-CH1-hinge-CH2-CH3; the second monomer comprises a heavy chain with a C-terminal scIL15-IL15Rα complex e.g. VH-CH1-hinge-CH2-CH3-domain linker-IL-15 variant-domain linker-IL-15Rα(sushi) domain; and the third (and fourth) monomer are light chains, VL-CL. The “Fab-Fc-IL15 x Fab-Fc” format (FIG. 25Q) comprises three monomers (although the fusion protein is a tetramer)—the first monomer comprises a heavy chain, VH-CH1-hinge-CH2-CH3; the second monomer comprises a heavy chain with a C-terminal IL15 i.e. VH-CH1-hinge-CH2-CH3-domain linker-IL-15 variant; and the third (and fourth) monomer are light chains, VL-CL. A similar format not shown here which may be referred to as the “Fab-Fc-IL15 x Fab-Fc-IL15” format has a C-terminal IL15 on the first monomer e.g. VH-CH1-hinge-CH2-CH3-domain linker IL-15 variant. The “Fab-Fc-IL15Rα+ncIL15 x Fab-Fc” format (FIG. 25R) comprises four monomers (although the heterodimeric fusion protein is a pentamer)—the first monomer comprises a heavy chain, VH-CH1-hinge-CH2-CH3; the second monomer comprises a heavy chain with a C-terminal IL-15Rα(sushi) domain e.g. VH-CH1-hinge-CH2-CH3-domain linker-IL-15Rα(sushi) domain; the third monomer is a variant IL-15 domain; and the fourth (and fifth) monomer are light chains, VL-CL. The “Fab-Fc-IL15+ncIL15Rα x Fab-Fc” format (FIG. 25S) comprises four monomers (although the heterodimeric fusion protein is a pentamer)—the first monomer comprises a heavy chain, VH-CH1-hinge-CH2-CH3; the second monomer comprises a heavy chain with a C-terminal variant IL-15 e.g. VH-CH1-hinge-CH2-CH3-domain linker-IL-15; the third monomer is a IL-15Rα(sushi) domain; and the fourth (and fifth) monomer are light chains, VL-CL. The “Fab-Fc-IL15 x Fab-Fc-IL15Rα” format (FIG. 25T) comprises three monomers (although the fusion protein is a tetramer)—the first monomer comprises a heavy chain with a C-terminal variant IL-15 e.g. VH-CH1-hinge-CH2-CH3-domain linker-IL-15; the second monomer comprises a heavy chain with a C-terminal IL15Rα(sushi) domain e.g. VH-CH1-hinge-CH2-CH3-domain linker-IL-15Rα(sushi) domain; and the third (and fourth) monomer are light chains, VL-CL. The “Fab-Fc-IL15Rα+dsIL15 x Fab-Fc” format (FIG. 25U) comprises four monomers (although the heterodimeric fusion protein is a pentamer)—the first monomer comprises a heavy chain, VH-CH1-hinge-CH2-CH3; the second monomer comprises a heavy chain with a C-terminal variant IL-15Rα(sushi) domain e.g., VH-CH1-hinge-CH2-CH3-domain linker-IL-15Rα(sushi) domain, where the IL-15Rα(sushi) domain has been engineered to contain a cysteine residue; the third monomer is a variant IL-15 domain, which has been engineered to contain a cysteine residue, such that the IL-15 complex is formed under physiological conditions; and the fourth (and fifth) monomer are light chains, VL-CL. The “Fab-Fc-IL15+dsIL15Rα x Fab-Fc” format (FIG. 25V) comprises four monomers (although the heterodimeric fusion protein is a pentamer)—the first monomer comprises a heavy chain, VH-CH1-hinge-CH2-CH3; the second monomer comprises a heavy chain with a C-terminal variant IL-15 domain e.g. VH-CH1-hinge-CH2-CH3-domain linker-IL-15 domain, where the variant IL-15 domain has been engineered to contain a cysteine residue; the third monomer is a variant IL-15Rα(sushi) domain, which has been engineered to contain a cysteine residue, such that the IL-15 complex is formed under physiological conditions; and the fourth (and fifth) monomer are light chains, VL-CL. The “Fab-Fc-IL15 x Fab-Fc-IL15Rα w/ ds” format (FIG. 25W) comprises three monomers (although the fusion protein is a tetramer)—the first monomer comprises a heavy chain with a C-terminal variant IL-15 e.g. VH-CH1-hinge-CH2-CH3-domain linker-IL-15, where the variant IL-15 domain has been engineered to contain a cysteine residue; the second monomer comprises a heavy chain with a C-terminal variant IL15Rα(sushi) domain e.g. VH-CH1-hinge-CH2-CH3-domain linker-IL-15Rα(sushi) domain, which has been engineered to contain a cysteine residue, such that the IL-15 complex is formed under physiological conditions; and the third (and fourth) monomer are light chains, VL-CL. The “Fab-Fc-IL15-Fc x Fab-IL15Rα-Fc” format (FIG. 25X) comprises four monomers forming a tetramer—The first monomer comprises a VH-CH1-[optional domain linker]-IL-15 variant-[optional domain linker]-CH2-CH3, with the second optional domain linker sometimes being the hinge domain; the second monomer comprises a VH-CH1-[optional domain linker]-IL-15Rα(sushi) domain-[optional domain linker]-CH2-CH3, with the second optional domain linker sometimes being the hinge domain; and the third (and fourth) monomers are light chains, VL-CL. The “Fab-Fc-IL15-Fc x Fab-IL15Rα-Fc w/ ds” format (FIG. 25Y) comprises four monomers forming a tetramer—the first monomer comprises a VH-CH1-[optional domain linker]-IL-15 variant-[optional domain linker]-CH2-CH3, with the second optional domain linker sometimes being the hinge domain, where the variant IL-15 domain has been engineered to contain a cysteine residue; the second monomer comprises a VH-CH1-[optional domain linker]-IL-15Rα(sushi) domain-[optional domain linker]-CH2-CH3, with the second optional domain linker sometimes being the hinge domain, where the variant IL-15Rα(sushi) has been engineered to contain a cysteine residue, such that the IL-15 complex is formed under physiological conditions; and the third (and fourth) monomers are light chains, VL-CL. The “Fab-IL15-Fc x Fab-IL15-Fc” format (FIG. 25Z) comprises four monomers forming a tetramer—the first and second monomer comprises a VH-CH1-[optional domain linker]-IL-15 variant-[optional domain linker]-CH2-CH3, with the second optional domain linker sometimes being the hinge domain; and the third (and fourth) monomers are light chains, VL-CL. The “Fab-scIL15Rα-IL15-Fc x Fab-Fc” format (FIG. 25AA) comprises four monomers forming a tetramer—the first monomer comprises a VH-CH1-[optional domain linker]-IL-15Rα(sushi) domain-domain linker-IL-15 variant-[optional domain linker]-CH2-CH3, with the second optional domain linker sometimes being the hinge domain; the second monomer comprises a VH-CH1-hinge-CH2-CH3; and the third (and fourth) monomers are light chains, VL-CL. The “Fab-scIL15-IL15Rα-Fc x Fab-Fc” format (FIG. 25BB) comprises four monomers forming a tetramer—the first monomer comprises a VH-CH1-[optional domain linker]-IL-15 variant-domain linker-IL-15Rα(sushi) domain-[optional domain linker]-CH2-CH3, with the second optional domain linker sometimes being the hinge domain; the second monomer comprises a VH-CH1-hinge-CH2-CH3; and the third (and fourth) monomers are light chains, VL-CL. The “Fab-IL15-Fc x Fab-Fc” format (FIG. 25CC) comprises four monomers forming a tetramer—the first monomer comprises a VH-CH1-[optional domain linker]-IL-15 variant-[optional domain linker]-CH2-CH3, with the second optional domain linker sometimes being the hinge domain; the second monomer comprises a VH-CH1-hinge-CH2-CH3; and the third (and fourth) monomers are light chains, VL-CL.

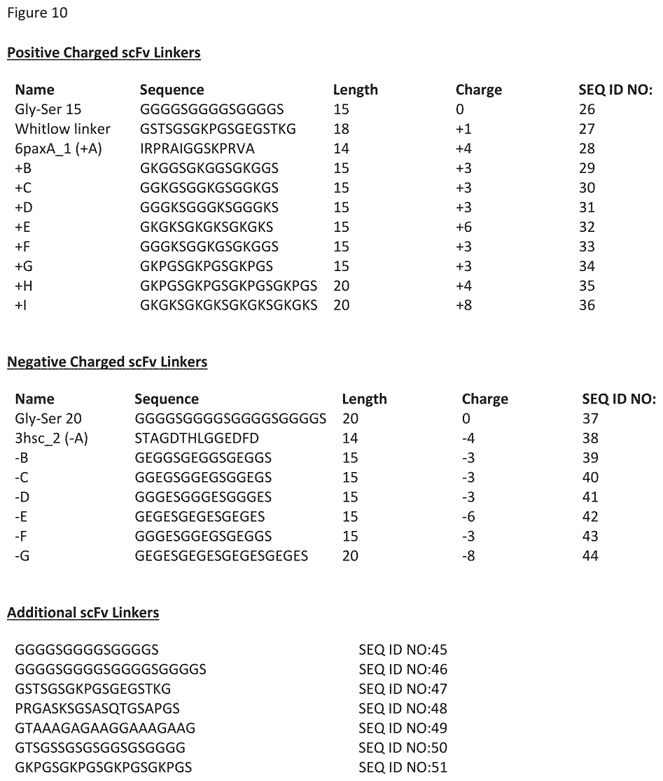

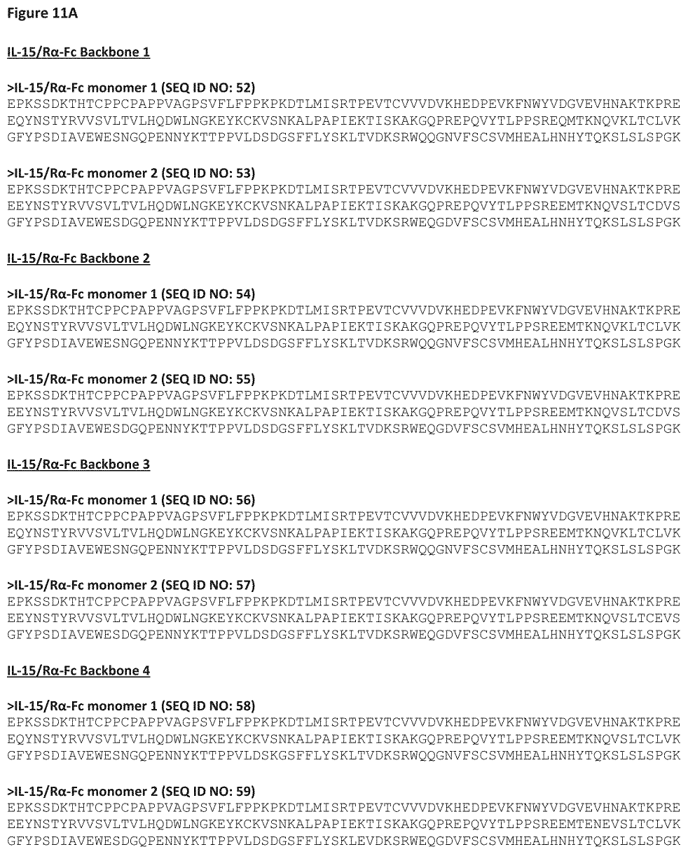

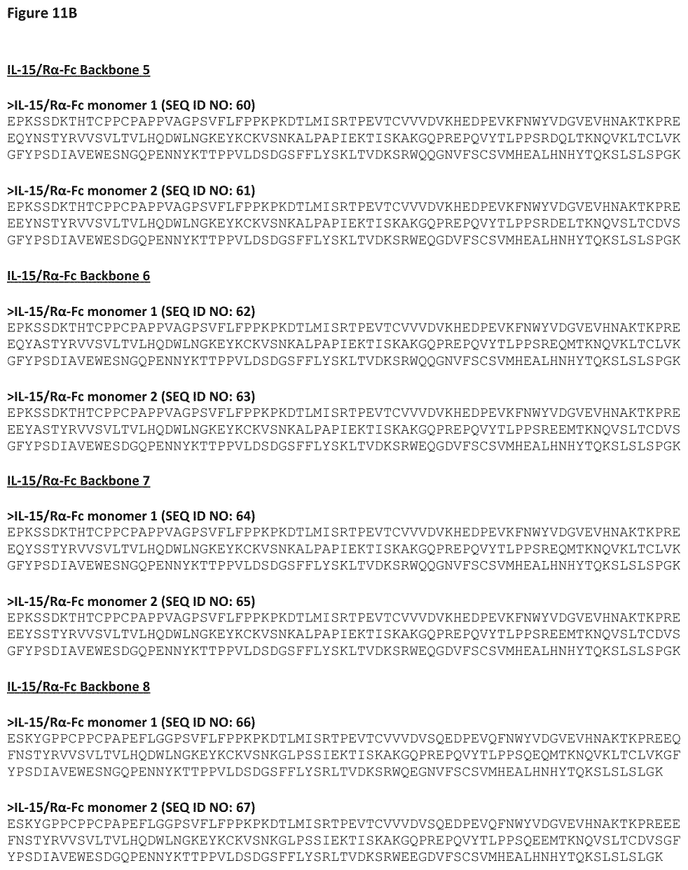

FIGS. 26A-26D depict sequences of illustrative ICOS-targeted IL-15/Rα-Fc fusion proteins of the “scIL-15/Rα x Fab” format. The CDRs are in bold. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. IL-15 and IL-15Rα(sushi) are underlined, linkers are double underlined (although as will be appreciated by those in the art, the linkers can be replaced by other linkers, some of which are depicted in FIGS. 9 and 10), and slashes (/) indicate the border(s) between IL-15, IL-15Rα, linkers, variable regions, and constant/Fc regions.

FIGS. 27A-27B depict the sequences of XENP26007 and XENP29481, a control RSV-targeted IL-15/Rα-Fc fusion. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. IL-15 and IL-15Rα(sushi) are italicized, linkers are double underlined (although as will be appreciated by those in the art, the linkers can be replaced by other linkers, some of which are depicted in FIGS. 9 and 10), and slashes (/) indicate the border(s) between IL-15, IL-15Rα, linkers, variable regions, and constant/Fc regions. As will be clear to those skilled in the art, each of the ICOS-targeted IL-15/Rα-Fc fusion proteins described can also include Xtend Fc (M428L/N434S).

FIGS. 28A-28B depict induction of A) CD8+ T cells and B) CD4+ T cells proliferation by ICOS-targeted IL-15/Rα-Fc fusions (and controls) as indicated by percentage proliferating cells (determined based on CFSE dilution). The data show that ICOS-targeted IL-15/Rα-Fc fusions are much more potent in inducing proliferation of both CD8+ and CD4+ T cells in comparison to untargeted IL-15/Rα-Fc fusion (as well as control RSV-targeted IL-15/Rα-Fc fusion). Experiment was performed using human PBMCs from donor 1.

FIGS. 29A-29B depict induction of A) CD8⁺CD45RA⁻ T cells and B) CD8⁺CD45RA⁺ T cells proliferation by ICOS-targeted IL-15/Rα-Fc fusions (and controls) as indicated by percentage proliferating cells (determined based on CFSE dilution). Experiment was performed using human PBMCs from donor 2.

FIGS. 30A-30B depict induction of A) CD8⁺CD45RA⁻ T cells and B) CD8⁺CD45RA⁺ T cells proliferation by ICOS-targeted IL-15/Rα-Fc fusions (and controls) as indicated by percentage proliferating cells (determined based on CFSE dilution). The data show that ICOS-targeted IL-15/Rα-Fc fusions are much more potent in inducing proliferation of CD8⁺CD45RA⁻ T cells in comparison to untargeted IL-15/Rα-Fc fusion (as well as control RSV-targeted IL-15/Rα-Fc fusion). Experiment was performed using human PBMCs from donor 1.

FIGS. 31A-31B depict induction of A) CD8⁺CD45RA⁻ T cells and B) CD8⁺CD45RA⁺ T cells proliferation by ICOS-targeted IL-15/Rα-Fc fusions (and controls) as indicated by cell counts. The data show that ICOS-targeted IL-15/Rα-Fc fusions are much more potent in inducing proliferation of CD8⁺CD45RA⁻ T cells in comparison to untargeted IL-15/Rα-Fc fusion (as well as control RSV-targeted IL-15/Rα-Fc fusion). Experiment was performed using human PBMCs from donor 1.

FIGS. 32A-32B depict induction of A) CD8⁺CD45RA⁻ T cells and B) CD8⁺CD45RA⁺ T cells proliferation by ICOS-targeted IL-15/Rα-Fc fusions (and controls) as indicated by cell counts. Experiment was performed using human PBMCs from donor 2.

FIGS. 33A-33B depict induction of A) CD4⁺CD45RA⁻ T cells and B) CD4⁺CD45RA⁺ T cells proliferation by ICOS-targeted IL-15/Rα-Fc fusions (and controls) as indicated by percentage proliferating cells (determined based on CFSE dilution). The data show that ICOS-targeted IL-15/Rα-Fc fusions are much more potent in inducing proliferation of CD4⁺CD45RA⁻ T cells and CD4⁺CD45RA⁺ T cells in comparison to untargeted IL-15/Rα-Fc fusion (as well as control RSV-targeted IL-15/Rα-Fc fusion). Experiment was performed using human PBMCs from donor 1.

FIGS. 34A-34B depict induction of A) CD4⁺CD45RA⁻ T cells and B) CD4⁺CD45RA⁺ T cells proliferation by ICOS-targeted IL-15/Rα-Fc fusions (and controls) as indicated by percentage proliferating cells (determined based on CFSE dilution). The data show that ICOS-targeted IL-15/Rα-Fc fusions are more potent in inducing proliferation of CD4⁺CD45RA⁻ T cells and CD4⁺CD45RA⁺ T cells in comparison to untargeted IL-15/Rα-Fc fusion (as well as control RSV-targeted IL-15/Rα-Fc fusion). Experiment was performed using human PBMCs from donor 2.

FIGS. 35A-35B depict induction of A) CD4⁺CD45RA⁻ T cells and B) CD4⁺CD45RA⁺ T cells proliferation by ICOS-targeted IL-15/Rα-Fc fusions (and controls) as indicated by cell counts. The data show that ICOS-targeted IL-15/Rα-Fc fusions are much more potent in inducing proliferation of CD4⁺CD45RA⁻ T cells and CD4⁺CD45RA⁺ T cells in comparison to untargeted IL-15/Rα-Fc fusion (as well as control RSV-targeted IL-15/Rα-Fc fusion). Experiment was performed using human PBMCs from donor 1.

FIGS. 36A-36B depict induction of A) CD4⁺CD45RA⁻ T cells and B) CD4⁺CD45RA⁺ T cells proliferation by ICOS-targeted IL-15/Rα-Fc fusions (and controls) as indicated by cell counts. The data show that ICOS-targeted IL-15/Rα-Fc fusions are much more potent in inducing proliferation of CD4⁺CD45RA⁻ T cells in comparison to untargeted IL-15/Rα-Fc fusion (as well as control RSV-targeted IL-15/Rα-Fc fusion). Experiment was performed using human PBMCs from donor 2.

FIGS. 37A-37B depict induction of NK cells proliferation by ICOS-targeted IL-15/Rα-Fc fusions (and controls) as indicated A) percentage proliferating cells (determined based on CFSE dilution) and B) by cell counts. The data show that ICOS-targeted IL-15/Rα-Fc fusions are much less potent in inducing proliferation of NK cells in comparison to untargeted IL-15/Rα-Fc fusion (as well as control RSV-targeted IL-15/Rα-Fc fusion). Experiment was performed using human PBMCs from donor 1.

FIGS. 38A-38B depicts induction of NK cells proliferation by ICOS-targeted IL-15/Rα-Fc fusions (and controls) as indicated A) percentage proliferating cells (determined based on CFSE dilution) and B) by cell counts. The data show that ICOS-targeted IL-15/Rα-Fc fusions are much less potent in inducing proliferation of NK cells in comparison to untargeted IL-15/Rα-Fc fusion (as well as control RSV-targeted IL-15/Rα-Fc fusion). Experiment was performed using human PBMCs from donor 2.

FIGS. 39A-39B depicts activation of A) CD8⁺CD45RA⁻ T cells and B) CD8⁺CD45RA⁺ T cells following incubation with ICOS-targeted IL-15/Rα-Fc fusions (and controls) as indicated by percentage cells expressing CD25. The data show that ICOS-targeted IL-15/Rα-Fc fusions appear to upregulate CD25 in both CD8⁺CD45RA⁻ T cells and CD8⁺CD45RA⁺ T cells more potently on CD4⁺CD45RA⁻ T cells in comparison to untargeted IL-15/Rα-Fc fusion (as well as control RSV-targeted IL-15/Rα-Fc fusion). Experiment was performed using human PBMCs from donor 1.

FIGS. 40A-40B depict activation of A) CD8⁺CD45RA⁻ T cells and B) CD8⁺CD45RA⁺ T cells following incubation with ICOS-targeted IL-15/Rα-Fc fusions (and controls) as indicated by percentage cells expressing CD25. The data show that ICOS-targeted IL-15/Rα-Fc fusions appear to upregulate CD25 in both CD8⁺CD45RA⁻ T cells and CD8⁺CD45RA⁺ T cells more potently in comparison to untargeted IL-15/Rα-Fc fusion (as well as control RSV-targeted IL-15/Rα-Fc fusion). Experiment was performed using human PBMCs from donor 2.

FIGS. 41A-41B depict activation of A) CD8⁺CD45RA⁻ T cells and B) CD8⁺CD45RA⁺ T cells following incubation with ICOS-targeted IL-15/Rα-Fc fusions (and controls) as indicated by CD25 MFI. Experiment was performed using human PBMCs from donor 1.

FIGS. 42A-42B depict activation of A) CD8⁺CD45RA⁻ T cells and B) CD8⁺CD45RA⁺ T cells following incubation with ICOS-targeted IL-15/Rα-Fc fusions (and controls) as indicated by CD25 MFI. Experiment was performed using human PBMCs from donor 2.

FIGS. 43A-43B depict activation of A) CD4⁺CD45RA⁻ T cells and B) CD4⁺CD45RA⁺ T cells following incubation with ICOS-targeted IL-15/Rα-Fc fusions (and controls) as indicated by percentage cells expressing CD25. The data show that ICOS-targeted IL-15/Rα-Fc fusions upregulate CD25 more potently on CD4⁺CD45RA⁻ T cells in comparison to untargeted IL-15/Rα-Fc fusion (as well as control RSV-targeted IL-15/Rα-Fc fusion). Experiment was performed using human PBMCs from donor 1.

FIGS. 44A-44B depict activation of A) CD4⁺CD45RA⁻ T cells and B) CD4⁺CD45RA⁺ T cells following incubation with ICOS-targeted IL-15/Rα-Fc fusions (and controls) as indicated by percentage cells expressing CD25. The data show that ICOS-targeted IL-15/Rα-Fc fusions upregulate CD25 more potently on CD4⁺CD45RA⁻ T cells and CD4⁺CD45RA⁺ T cells in comparison to untargeted IL-15/Rα-Fc fusion (as well as control RSV-targeted IL-15/Rα-Fc fusion). Experiment was performed using human PBMCs from donor 2.

FIGS. 45A-45B depicts activation of A) CD4⁺CD45RA⁻ T cells and B) CD4⁺CD45RA⁺ T cells following incubation with ICOS-targeted IL-15/Rα-Fc fusions (and controls) as indicated by CD25 MFI. Experiment was performed using human PBMCs from donor 1.

FIGS. 46A-46B depict activation of A) CD4⁺CD45RA⁻ T cells and B) CD4⁺CD45RA⁺ T cells following incubation with ICOS-targeted IL-15/Rα-Fc fusions (and controls) as indicated by CD25 MFI. The data show that ICOS-targeted IL-15/Rα-Fc fusions upregulate CD25 more potently on CD4⁺CD45RA⁻ T cells and CD4⁺CD45RA⁺ T cells in comparison to untargeted IL-15/Rα-Fc fusion (as well as control RSV-targeted IL-15/Rα-Fc fusion). Experiment was performed using human PBMCs from donor 2.

FIGS. 47A-47B depict activation of HLA-DR on A) CD8⁺CD45RA⁻ T cells and B) CD8⁺CD45RA⁺ T cells following incubation with ICOS-targeted IL-15/Rα-Fc fusions (and controls) as indicated by percentage cells expressing HLA-DR. Experiment was performed using human PBMCs from donor 1.

FIGS. 48A-48B depict activation of HLA-DR on A) CD8⁺CD45RA⁻ T cells and B) CD8⁺CD45RA⁺ T cells following incubation with ICOS-targeted IL-15/Rα-Fc fusions (and controls) as indicated by percentage cells expressing HLA-DR. Experiment was performed using human PBMCs from donor 2.

FIGS. 49A-49B depict activation of A) CD8⁺CD45RA⁻ T cells and B) CD8⁺CD45RA⁺ T cells following incubation with ICOS-targeted IL-15/Rα-Fc fusions (and controls) as indicated by HLA-DR MFI. Experiment was performed using human PBMCs from donor 1.

FIGS. 50A-50B depict activation of A) CD8⁺CD45RA⁻ T cells and B) CD8⁺CD45RA⁺ T cells following incubation with ICOS-targeted IL-15/Rα-Fc fusions (and controls) as indicated by HLA-DR MFI. Experiment was performed using human PBMCs from donor 2.

FIGS. 51A-51B depict activation of A) CD4⁺CD45RA⁻ T cells and B) CD4⁺CD45RA⁺ T cells following incubation with ICOS-targeted IL-15/Rα-Fc fusions (and controls) as indicated by percentage cells expressing HLA-DR. Experiment was performed using human PBMCs from donor 1.

FIGS. 52A-52B depict activation of A) CD4⁺CD45RA⁻ T cells and B) CD4⁺CD45RA⁺ T cells following incubation with ICOS-targeted IL-15/Rα-Fc fusions (and controls) as indicated by percentage cells expressing HLA-DR. Experiment was performed using human PBMCs from donor 2.

FIGS. 53A-53B depict activation of A) CD4⁺CD45RA⁻ T cells and B) CD4⁺CD45RA⁺ T cells following incubation with ICOS-targeted IL-15/Rα-Fc fusions (and controls) as indicated by HLA-DR MFI. Experiment was performed using human PBMCs from donor 1.

FIGS. 54A-54B depict activation of A) CD4⁺CD45RA⁻ T cells and B) CD4⁺CD45RA⁺ T cells following incubation with ICOS-targeted IL-15/Rα-Fc fusions (and controls) as indicated by HLA-DR MFI. Experiment was performed using human PBMCs from donor 2.

FIG. 55 depicts the sequences of XENP22853, an IL-15/Rα-heteroFc fusion comprising a wild-type IL-15 and Xtend Fc (M428L/N434S) variant. IL-15 and IL-15Rα(sushi) are underlined, linkers are double underlined (although as will be appreciated by those in the art, the linkers can be replaced by other linkers, some of which are depicted in the Figures, and slashes (/) indicate the border(s) between IL-15, IL-15Rα, linkers, and constant/Fc regions.

FIG. 56 depicts the sequences of XENP4113, an IL-15/Rα-heteroFc fusion comprising a IL-15(N4D/N65D) variant and Xtend Fc (M428L/N434S) variant. IL-15 and IL-15Rα(sushi) are underlined, linkers are double underlined (although as will be appreciated by those in the art, the linkers can be replaced by other linkers, some of which are depicted in the Figures, and slashes (/) indicate the border(s) between IL-15, IL-15Rα, linkers, and constant/Fc regions.

FIG. 57 depicts the sequences of XENP24294, an scIL-15/Rα-Fc fusion comprising a IL-15(N4D/N65D) variant and Xtend Fc (M428L/N434S) substitution. IL-15 and IL-15Rα(sushi) are underlined, linkers are double underlined (although as will be appreciated by those in the art, the linkers can be replaced by other linkers, some of which are depicted in the Figures, and slashes (/) indicate the border(s) between IL-15, IL-15Rα, linkers, and constant/Fc regions.

FIG. 58 depicts the sequences of XENP24306, an IL-15/Rα-heteroFc fusion comprising a IL-15(D30N/E64Q/N65D) variant and Xtend Fc (M428L/N434S) substitution. IL-15 and IL-15Rα(sushi) are underlined, linkers are double underlined (although as will be appreciated by those in the art, the linkers can be replaced by other linkers, some of which are depicted in the Figures, and slashes (/) indicate the border(s) between IL-15, IL-15Rα, linkers, and constant/Fc regions.

FIG. 59 depicts the serum concentration of the indicated test articles over time in cynomolgus monkeys following a first dose at the indicated relative concentrations.

FIGS. 60A-60B depict the variable heavy and variable light chains for additional illustrative ICOS ABDs. The variable heavy chains, variable lights, and six CDRs of such ICOS ABDs find use in the fusion proteins and antibodies described herein. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the V_{H} and V_{L} domains using other numbering systems.

FIGS. 61A-61P depict sequences of additional illustrative ICOS-targeted IL-15/Rα-Fc fusion proteins of the “scIL-15/Rα x Fab” format. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. IL-15 and IL-15Rα(sushi) are underlined, linkers are double underlined (although as will be appreciated by those in the art, the linkers can be replaced by other linkers, some of which are depicted in FIGS. 9 and 10), and slashes (/) indicate the border(s) between IL-15, IL-15Rα, linkers, variable regions, and constant/Fc regions. As will be clear to those skilled in the art, each of the ICOS-targeted IL-15/Rα-Fc fusion proteins described can also include Xtend Fc (M428L/N434S).

FIGS. 62A-62G depict anti-ICOS antibodies. The variable heavy chains, variable light chains, and 6 CDRs of such antibodies find use in the fusion proteins and antibodies provided herein. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems.

FIGS. 63A-63G depict anti-ICOS antibodies. The variable heavy, variable light chains, and 6 CDRs of such antibodies find use in the fusion proteins and antibodies provided herein. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems.

FIGS. 64A-64M depict ICOS antigen binding domains (ABDs). The variable heavy, variable light chains, and 6 CDRs of such ICOS binding domains find use in the fusion proteins and antibodies provided herein. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems.

### DETAILED DESCRIPTION

### I. Definitions

In order that the application may be more completely understood, several definitions are set forth below. Such definitions are meant to encompass grammatical equivalents.

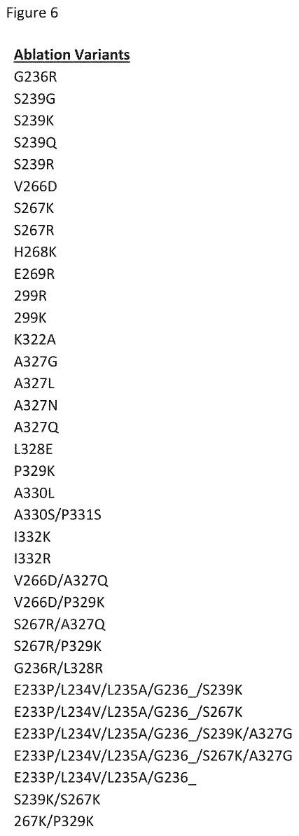

By “ablation” herein is meant a decrease or removal of activity. Thus for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. Of particular use in the ablation of FcγR binding are those shown in FIG. 6. However, unless otherwise noted, the Fc monomers of the invention retain binding to the FcRn receptor.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the invention ablate ADCC activity entirely.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

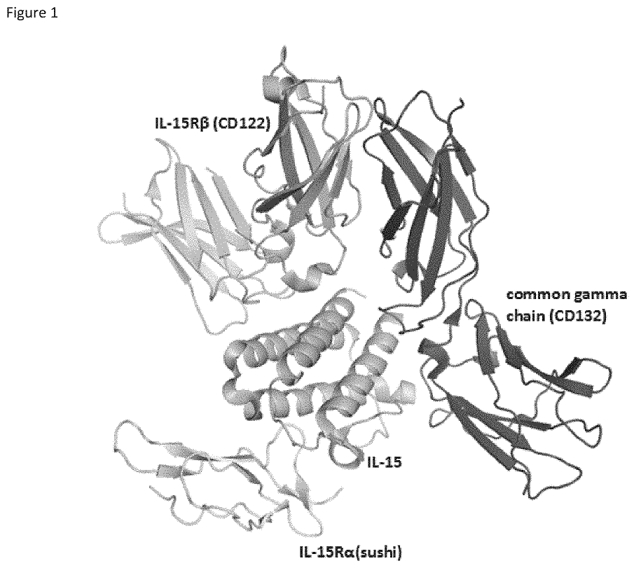

By “antigen binding domain” or “ABD” herein is meant a set of six Complementary Determining Regions (CDRs) that, when present as part of a polypeptide sequence, specifically binds a target antigen as discussed herein. Thus, a “ICOS antigen binding domain” binds a human ICOS antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used (e.g., from FIG. 1 of U.S. 62/353,511).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233#, E233( ) or E233del designates a deletion of glutamic acid at position 233. Additionally, EDA233− or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it.

Accordingly, by “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference). The modification can be an addition, deletion, or substitution. Substitutions can include naturally occurring amino acids and, in some cases, synthetic amino acids. Examples include U.S. Pat. No. 6,586,207; WO 98/48032; WO 03/073238; U52004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein.

By “Fv” or “Fv fragment” or “Fv region” as used herein is meant a polypeptide that comprises the VL and VH domains of a single antibody. As will be appreciated by those in the art, these generally are made up of two chains, or can be combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh).

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

By “FcRn” or “neonatal Fc Receptor” as used herein is meant a protein that binds the IgG antibody Fc region and is encoded at least in part by an FcRn gene. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers of the invention retain binding to the FcRn receptor (and, as noted below, can include amino acid variants to increase binding to the FcRn receptor).

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

By “Fc” or “Fc region” or “Fc domain” as used herein is meant the polypeptide comprising the constant region of an antibody excluding the first constant region immunoglobulin domain (e.g., CH1) and in some cases, part of the hinge. For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In the embodiments herein, when a scFv or IL-15 complex is attached to an Fc domain, it is the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcγR receptors or to the FcRn receptor, and to enable heterodimer formation and purification, as outlined herein.

By “heavy constant region” herein is meant the CH1-hinge-CH2-CH3 portion of an antibody.

By “Fc fusion protein” or “immunoadhesin” herein is meant a protein comprising an Fc region, generally linked (optionally through a linker moiety, as described herein) to a different protein, such as to IL-15 and/or IL-15Rα(sushi), as described herein. In some instances, two Fc fusion proteins can form a homodimeric Fc fusion protein or a heterodimeric Fc fusion protein with the latter being preferred. In some cases, one monomer of the heterodimeric Fc fusion protein comprises an Fc domain alone (e.g., an empty Fc domain) and the other monomer is a Fc fusion, comprising a variant Fc domain and a protein domain, such as a receptor, ligand or other binding partner.

By “position” as used herein is meant a location in the sequence of a protein. Positions may be numbered sequentially, or according to an established format, for example the EU index for antibody numbering.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses the target antigen, in this case, ICOS.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The ICOS targeted heterodimeric fusion proteins of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins having different binding specificities. “Recombinant” means the proteins are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Specific binding” or “specifically binds to” or is “specific for” a particular antigen or an epitope (in this case, human ICOS) means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸M, at least about 10⁻⁹M, alternatively at least about 10⁻¹⁰ M, at least about 10⁻¹¹M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay.

### II. Introduction

The invention provides heterodimeric fusion proteins that contain two functionalities, an IL-15 function and an ICOS antigen binding domain. As shown in FIG. 25, these fusion proteins can take on a number of different formats. In general, the proteins of the invention are multimeric, in that they contain two or more separate polypeptide chains that self-associate to form multimeric (including heterodimeric) protein complexes.

In some aspects, the heterodimeric fusion proteins contain an IL-15/IL-15Rα complex on one side and an anti-human ICOS antigen binding domain on the other. Thus, the heterodimeric fusion proteins of the invention can bind to the checkpoint ICOS antigen and can complex with the common gamma chain (yc; CD132) and/or the IL-2 receptor β-chain (IL-2Rβ; CD122). In general, the heterodimeric fusion proteins of the invention have three functional components: an IL-15/IL-15Rα(sushi) component, generally referred to herein as an “IL-15 complex”, an ICOS ABD (referred to as “ICOS ABD” interchangeably) component (which serves as a “targeting” moiety by bringing the fusion protein to a cell expressing ICOS), and an Fc component, each of which can take different forms and each of which can be combined with the other components in any configuration.

In some cases, as is more fully discussed below, the fusion proteins of the invention do not include a sushi domain; rather, the IL-15 variant has been engineered to reduce or ablate the ability of IL-15 to bind to the IL-15 receptor and in particular the sushi domain.

Additionally, in some cases, the IL-15 component is wild-type human IL-15.

In general, as is more fully described herein, the fusion proteins of the invention are heterodimeric fusion proteins that are based on the association of antibody Fc domains. That is, by using two different variant Fc domains that have been engineered to favor the formation of heterodimers over homodimers, the heterodimeric fusion proteins are formed. In this case, one of the variant Fc domains is fused to an IL-15/Rα complex (or an IL-15 variant that does not associate with the sushi domain) and the other has an ICOS ABD as more fully outlined herein. By including optional pI variants, the heterodimers can be more easily purified away from the homodimers. Additionally, the inclusion of ablation variants eliminates the effector functions of the Fc domains.

A. IL-15/IL-15Rα(Sushi) Domains

As shown in the figures, the IL-15/Rα complex can take several forms. As stated above, the IL-15 protein on its own is less stable than when complexed with the IL-15Rα protein. As is known in the art, the IL-15Rα protein contains a “sushi domain”, which is the shortest region of the receptor that retains IL-15 binding activity. Thus, while heterodimeric fusion proteins comprising the entire IL-15Rα protein can be made, preferred embodiments herein include complexes that just use the sushi domain, the sequence of which is shown in the figures.

Accordingly, the IL-15/Rα complex generally comprises the IL-15 protein and the sushi domain of IL-15Rα (unless otherwise noted that the full length sequence is used, “IL-15Rα”, “IL-15Rα(sushi)”, “IL-15RA” and “sushi” are used interchangeably throughout). When complexed together, the nomenclature is depicted with a “slash”, “/”, as “IL-15/Rα”, meaning that there is an IL-15 domain and an IL-15Rα domain present.

Importantly, the IL-15 component is generally engineered to reduce its potency. In many embodiments, the wild-type IL-15 is too potent and can cause undesirable toxicity. Accordingly, the IL-15 component of the IL-15/Rα complex can have one or more amino acid substitutions that result in decreased activity. Various amino acid substitutions were made (see FIG. 19) and tested (see FIG. 20A-FIG. 20C). Of particular interest in some embodiments are a double variant IL-15, N4D/N65D or D30N/N65D, or a triple variant IL-15, D30N/E64Q/N65D. Additional IL-15 variants are discussed below.

The targeted IL-15/IL-15Rα heterodimeric fusion proteins of the present invention include an IL-15/IL-15 receptor alpha (IL-15Rα)-Fc fusion monomer; reference is made to US2018/0118828, filed 16, October 2017, U.S. Ser. No. 62/408,655, filed on Oct. 14, 2016, U.S. Ser. No. 62/416,087, filed on October Nov. 1, 2016, U.S. Ser. No. 62/443,465, filed on Jan. 6, 2017, U.S. Ser. No. 62/477,926, filed on Mar. 28, 2017, and U.S. Ser. No. 62/659,571, filed on Apr. 18, 2018, hereby incorporated by reference in their entirety and in particular for the sequences outlined therein. In some cases, the IL-15 and IL-15 receptor alpha (IL-15Rα) protein domains are in different orientations. Exemplary embodiments of IL-15/IL-15Rα-Fc fusion monomers are provided in XENP21480 (chain 1; FIG. 64A), XENP22022 (chain 1, FIG. 64D), XENP22112, (chains 1 and 3; FIG. 64E), XENP22641 (chains 2 and 4; FIG. 64F), XENP22642, (chains 1 and 4; FIG. 64H) and XENP22644 (chains 1 and 4; FIG. 64I) as described, for example, in US 2018/0118828.

1. IL-15 Variants that Associate with IL-15(Sushi)

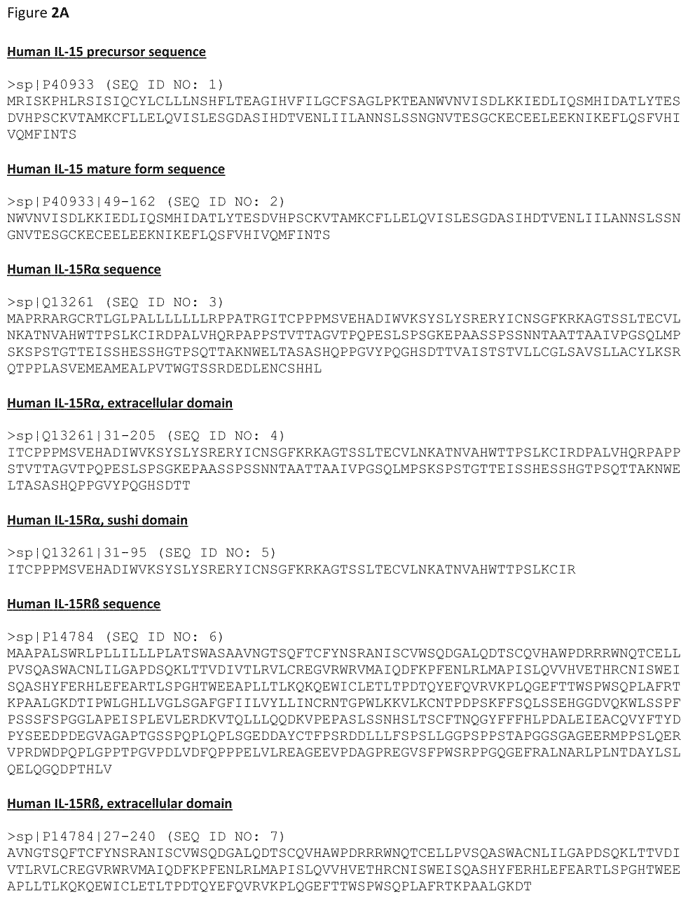

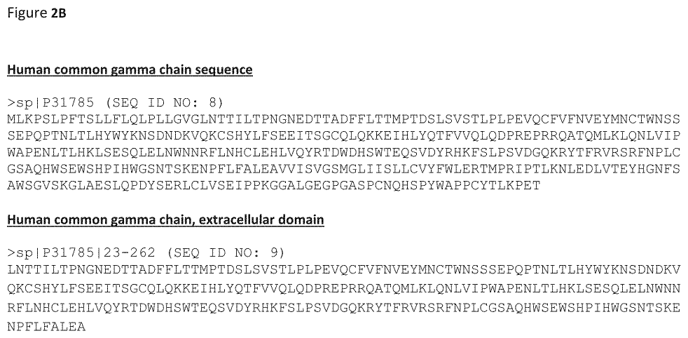

Of particular use in many embodiments are IL-15 variants that retain the ability to bind or associate with the IL-15 receptor alpha (e.g. the sushi domain) but have reduced potency as outlined below. While in some cases the human wild-type IL-15 protein can be used (e.g. the amino acid sequence set forth in NCBI Ref. Seq. No. NP 000576.1 as shown in FIG. 2, with the original coding sequence of human IL-15 is set forth in NCBI Ref. Seq. No. NM_000585), in many cases, amino acid modifications are preferred as outlined herein. An exemplary IL-15 protein of the Fc fusion heterodimeric fusion protein outlined herein can have the amino acid sequence of SEQ ID NO:2 or amino acids 49-162 of SEQ ID NO:1. In some embodiments, the IL-15 protein has at least 90%, e.g., 90%, 91%, 92%, 93%, 94%, 95%, 96%, 97%, 98%, 99%, or more sequence identity to SEQ ID NO:2.

Furthermore, in some embodiments, the IL-15 human protein is engineered to confer decreased potency as is generally described in PCT/US2019/028107, hereby incorporated by reference in its entirety. That is, as described therein, reduction in potency of IL-15 in the heterodimeric fusion proteins of the invention (optionally with and without Xtend-Fc substitutions described herein such as M428L/N434S or M428L/N434A or others described below) can enhance both pharmacodynamics and pharmacokinetics in subjects that are administered such proteins. Similarly, as shown in Example 7 of PCT/US2019/028107, that reduced potency IL-15/Rα-Fc variants such as XENP22821 can expand lymphocyte counts for a greater duration than wild-type IL-15/Rα-Fc fusion proteins described therein such as XENP20818. Notably, XENP23343, the Xtend-analog of XENP22821, further enhanced the duration of lymphocyte expansion beyond XENP22821. In addition, the reduction in potency of IL-15 can improve therapeutic index (i.e. enable higher dosing with less toxicity). As illustrated in the Example 8 of PCT/US2019/028107 for an “untargeted molecule” such as XmAb24306, IL-15/Rα-Fc fusion proteins such as those incorporated herein can overcome Treg suppression induced effector T cell proliferation.

Accordingly, the present invention provides a number of suitable IL-15 amino acid variants that confer reduced potency and increased pharmokinetics, including, but not limited to, variant IL-15 proteins comprising amino acid substitution(s) selected from the group of N1D; N4D; D8N; D30N; D61N; E64Q; N65D; Q108E; N1D/N4D/D8N; N1D/N4D/N65D; N1D/D30N; N1D/D61N; N1D/D61N/E64Q/Q108E; N1D/E64Q; N1D/N65D; N1D/Q108E; N4D; N4D/D30N; N4D/D61N; N4D/D61N/N65D; N4D/D61N/E64Q/Q108E; N4D/E64Q; N4D/N65D; D8N/D61N; D8N/E64Q; D30N/E64Q; D30N/N65D; D30N/E64Q/N65D; D30N/Q180E; D61N/E64Q/N65D; E64Q; E64Q/N65D; E64Q/Q108E; and N65D/Q108E.

In some embodiments, the IL-15 protein has the amino acid sequence set forth in SEQ ID NO:2 except with the amino acid substitution N72D. In other embodiments, the IL-15 protein has the amino acid sequence of SEQ ID NO:2 except with one or more amino acid substitutions selected from the group consisting of C42S, L45C, Q48C, V49C, L52C, E53C, E87C, and E89C. In some aspects, the IL-15 protein has one or more amino acid substitutions selected from the group consisting of N1D, N4D, D8N, D30N, D61N, E64Q, N65D, and Q108E. In other embodiments, the amino acid substitutions are N4D/N65D. In certain embodiments, the amino acid substitutions are D30N/N65D. In some embodiments, the amino acid substitution is Q108E. In certain embodiments, the amino acid substitution is N65D. In other embodiments, the amino acid substitutions are D30N/E64Q/N65D. In certain embodiments, the amino acid substitution is N65D. In some instances, the amino acid substitutions are N1D/N65D. In some instances, the amino acid substitutions are D30N/N65D. Optionally, the IL-15 protein also has an N72D substitution. The IL-15 protein of the Fc fusion protein can have 1, 2, 3, 4, 5, 6, 7, 8 or 9 amino acid substitutions. In some embodiments, the IL-15 protein of the Fc fusion protein comprises a D30N substitution. In some embodiments, the IL-15 protein of the Fc fusion protein comprises a N65D substitution. In some embodiments, the IL-15 protein of the Fc fusion contains one or more amino acid substitutions at the IL-15:CD132 interface. In certain embodiments, the Fc fusion protein described herein induces proliferation of NK cells and CD8+ T cells.

2. IL-15 Variants that do not Associate with IL-15Rα

In some cases, variant human IL-15 proteins are used that do not self-associate with the IL-15 receptor and in particular the sushi domain. As generally described in U.S. Pat. No. 11,059,876 (hereby expressly incorporated by reference and specifically for the variants and sequences outlined in Tables 3, 4 and 6), IL-15 variants can be made that have decreased or no binding to the IL-15Rα (also referred to as CD215) and optionally reduced binding (as compared to wild type) to its signaling receptor (comprised of the IL-2 receptor beta (CD122) and the common gamma chain (CD132)). Accordingly, specific variants of human IL-15 that do not associate with the IL-15Rα component, include, but are not limited to, N1G/D30N/E46G/V49R/E64Q (referred to in U.S. Pat. No. 11,059,876 as the “M2” construct), D22N/Y26F/E46Q/E53Q/E89Q/E93Q (referred to in U.S. Pat. No. 11,059,876 as “NQ”) and V49R/E46G/N1A/D30N, referred to in U.S. Pat. No. 11,059,876 as “M1”).

3. IL-15RA Components

As will be appreciated by those in the art, both wild-type human IL-15Rα or variants thereof can be used in the present invention. With the exception of the use of IL-15 variants that ablate binding to the sushi domain, as outlined above, when IL-15 variants are used, they retain the ability to bind to the sushi domain, including the wild type sushi domain. In some embodiments, the human IL-15 receptor alpha (IL-15Rα) protein has the amino acid sequence set forth in NCBI Ref. Seq. No. NP 002180.1 or SEQ ID NO:3. In some cases, the coding sequence of human IL-15Rα is set forth in NCBI Ref. Seq. No. NM_002189.3. An exemplary the IL-15Rα protein of the Fc fusion heterodimeric fusion protein outlined herein can comprise or consist of the sushi domain of SEQ ID NO:3 (e.g., amino acids 31-95 of SEQ ID NO:3), or in other words, the amino acid sequence of SEQ ID NO:4. In some embodiments, the IL-15Rα protein has the amino acid sequence of SEQ ID NO:4 and an amino acid insertion selected from the group consisting of D96, P97, A98, D96/P97, D96/C97, D96/P97/A98, D96/P97/C98, and D96/C97/A98, wherein the amino acid position is relative to full-length human IL-15Rα protein or SEQ ID NO:3. For instance, amino acid(s) such as D (e.g., Asp), P (e.g., Pro), A (e.g., Ala), DP (e.g., Asp-Pro), DC (e.g., Asp-Cys), DPA (e.g., Asp-Pro-Ala), DPC (e.g., Asp-Pro-Cys), or DCA (e.g., Asp-Cys-Ala) can be added to the C-terminus of the IL-15Rα protein of SEQ ID NO:4. In some embodiments, the IL-15Rα protein has the amino acid sequence of SEQ ID NO:4 and one or more amino acid substitutions selected from the group consisting of K34C, A37C, G38C, 540C, and L42C, wherein the amino acid position is relative to SEQ ID NO:4. The IL-15Rα protein can have 1, 2, 3, 4, 5, 6, 7, 8 or more amino acid mutations (e.g., substitutions, insertions and/or deletions).

4. IL-15/RA Complexes

As outlined herein, in formats including a sushi domain, the IL-15 variants and the sushi domain can be complexed in a variety of ways, as generally shown in FIGS. 14 and 25, and discussed below in Section III.

In some embodiments, as shown in FIG. 14C, for example, the IL-15 protein and the IL-15Rα(sushi) are not covalently attached, but rather are self-assembled through regular ligand-ligand interactions. As is more fully described herein, it can be either the IL-15 domain or the sushi domain that is covalently linked to the Fc domain (generally using an optional domain linker). Again, of particular use in this embodiment are a double variant, IL-15 N4D/N65D or D30N/N65D, or a triple variant IL-15, D30N/E64Q/N65D, used with a wild-type sushi domain.

In alternative embodiments, the variant IL-15 can be complexed (linked) to the sushi domain using a domain linker, such that they are covalently attached as generally shown in FIG. 14B; this figure depicts the sushi domain as the N-terminal domain, although this can be reversed. Again, of particular use in this embodiment are a double variant IL-15, N4D/N65D or D30N/N65D, or a triple variant IL-15, D30N/E64Q/N65D, used with a wild type sushi domain.

In some cases, each of the IL-15 variant and IL-15Rα(sushi) domain are engineered to contain a cysteine amino acid, that forms a disulfide bond to form the complex, with either the IL-15 domain or the sushi domain being covalently attached (using an optional domain linker) to the Fc domain. Again, of particular use in this embodiment are a double variant IL-15, N4D/N65D or D30N/N65D, (additionally including an amino acid substitution to cysteine), or a triple variant IL-15, D30N/E64Q/N65D (additionally including an amino acid substitution to cysteine), used with a sushi domain also comprising an amino acid substitution to provide a cysteine.

Additional particular embodiments are outlined below.

B. Anti-ICOS Components

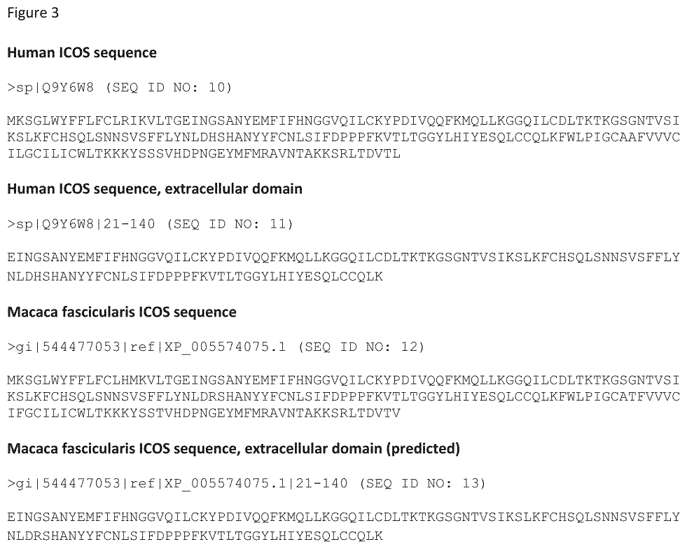

The heterodimeric fusion proteins of the invention contain some antibody components, including antigen binding domains that bind to human ICOS, the sequence of which is shown in FIG. 3.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to antibodies or antibody fragments (antibody monomers) that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

In addition, many of the monomer sequences herein have at least one the cysteines at position 220 replaced by a serine, to reduce disulfide formation. Specifically included within the sequences herein are one or both of these cysteines replaced (C220S).

Thus, “isotype” as used herein is meant any of the subclasses of immunoglobulins defined by the chemical and antigenic characteristics of their constant regions.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat+ | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat. As shown herein and described below, the pI variants can be in one or more of the CH regions, as well as the hinge region, discussed below.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230. As noted herein, pI variants can be made in the hinge region as well.

The light chain generally comprises two domains, the variable light domain (containing the light chain CDRs and together with the variable heavy domains forming the Fv region), and a constant light chain region (often referred to as CL or Cκ).

Another region of interest for additional substitutions, outlined herein, is the Fc region.

Thus, the present invention provides different antibody domains. As described herein and known in the art, the heterodimeric antibodies of the invention comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

As generally outlined herein, the heterodimeric fusion proteins of the invention include an Fv that binds human ICOS. This Fv, or anti-ICOS component (the anti-ICOS antigen binding domain or ABD) of the invention is generally a set of 6 CDRs and/or a variable heavy domain and a variable light domain that form an Fv domain that can bind human ICOS. As described herein, there are a number of different formats that can be used, generally either by using a scFv or a Fab as outlined herein.

In certain embodiments, the ABDs of the invention comprise a heavy chain variable region with frameworks from a particular germline heavy chain immunoglobulin gene and/or a light chain variable region from a particular germline light chain immunoglobulin gene. For example, such ABDs may comprise or consist of a human ABD comprising heavy or light chain variable regions that are “the product of” or “derived from” a particular germline sequence. An ABD that is “the product of” or “derived from” a human germline immunoglobulin sequence can be identified as such by comparing the amino acid sequence of the ABD to the amino acid sequences of human germline immunoglobulins and selecting the human germline immunoglobulin sequence that is closest in sequence (i.e., greatest % identity) to the sequence of the ABD. An ABD that is “the product of” or “derived from” a particular human germline immunoglobulin sequence may contain amino acid differences as compared to the germline sequence, due to, for example, CDRs, naturally-occurring somatic mutations or intentional introduction of site-directed mutation. However, a humanized ABD typically is at least 90% identical in amino acids sequence to an amino acid sequence encoded by a human germline immunoglobulin gene and contains amino acid residues that identify the ABD as being derived from human sequences when compared to the germline immunoglobulin amino acid sequences of other species (e.g., murine germline sequences). In certain cases, a humanized ABD may be at least 95%, 96%, 97%, 98%, or 99%, or even at least 96%, 97%, 98%, or 99% identical in amino acid sequence to the amino acid sequence encoded by the germline immunoglobulin gene. Typically, a humanized ABD derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized ABD may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention). In one embodiment, the parent ABD has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

As shown herein, the ICOS ABD can be in the form of either a Fab or an scFv, with a Fab format being particularly useful in many embodiments as generally shown in FIGS. 25A-CC.

In some embodiments the ICOS ABD is a scFv, wherein the VH and VL domains are joined using an scFv linker, which can be optionally a charged scFv linker. As will be appreciated by those in the art, the scFv can be assembled from N- to C-terminus as N-vh-scFv linker-vl-C or as N-vl-scFv linker-vh-C, with the C terminus of the scFv domain generally being linked to the hinge-CH2-CH3 Fc domain, wherein the hinge in this case serving as a domain linker. Suitable Fvs (including CDR sets and variable heavy/variable light domains) can be used in scFv formats or Fab formats are shown in the Figures.

As will further be appreciated by those in the art, all or part of the hinge (which can also be a wild type hinge from IgG1, IgG2 or IgG4 or a variant thereof, such as the IgG4 S241P or S228P hinge variant with the substitution proline at position 228 relative to the parent IgG4 hinge polypeptide (wherein the numbering S228P is according to the EU index and the S241P is the Kabat numbering)) can be used as the domain linker between the scFv and the CH2-CH3 domain, or a different domain linker such as depicted in the Figures can be used.

Alternatively, the ICOS ABD can be in the form of a Fab fragment. In this embodiment, the ABD is made up of a variable heavy domain, contributed by a heavy chain, and a variable light domain, contributed by a light chain. Suitable Fvs (including CDR sets and variable heavy/variable light domains) can be used in scFv formats or Fab formats are shown in the Figures.

In some embodiments, the anti-ICOS Fab components are the pairs of vh and vl domains as depicted in FIGS. 24, 60, and 62-64. In addition, suitable ICOS vh and vl domains can be found in WO/2018/045110, hereby incorporated by reference in its entirety and specifically for the sequences depicted in FIG. 19, FIG. 20 and FIG. 24, as well as SEQ ID NOs:27869-28086 from the sequence listing that are a number of ICOS Fab sequences (heavy chain VH1-CH1 and light chain VL1-CL) as indicated in the naming nomenclature. Any or all of these may find use in the present invention.

Accordingly, suitable human ICOS antigen binding domains include, but are not limited to, those listed in FIGS. 24, 60, and 62-64 (e.g., SEQ ID NOs:140-143, 179-242, 315-584) and the sequence listing (e.g., SEQ ID NOs:585-802).

In some embodiments, the ICOS antigen binding domain (ABD) comprises a variable heavy domain that comprises an amino acid sequence with at least about 95%, about 96%, about 97%, about 98%, or about 99%, sequence identity to the amino acid sequence of the variable heavy domains of a parent ICOS ABD. In some embodiments, the parent ICOS ABD is any of the ICOS ABDs set forth in in FIGS. 24, 60, and 62-64 (e.g., SEQ ID NOs:140-143, 179-242, 315-584) and the sequence listing (e.g., SEQ ID NOs:585-802). In some embodiments, the ICOS ABD retains the binding and/or functional activity of the patent ICOS ABD. In still further embodiments, the ICOS ABD comprises the variable heavy domain sequence of the parent ICOS ABD and has one or more amino acid substitutions, e.g., 1, 2, 3, 4, 5, 1-2, 1-3, 1-4 or 1-5 conservative amino acid substitutions in the heavy variable domain sequence. In yet further embodiments, the one or more amino acid substitutions fall within one or more framework regions of the variable heavy domain sequences of the parent ICOS ABD.

In particular embodiments, the ICOS ABD comprises a variable heavy domain sequence with at least about 95%, about 96%, about 97%, about 98%, or about 99% sequence identity to a variable heavy domain sequence of a parent ICOS ABD. In some embodiments, the parent ICOS ABD is any one of the ICOS ABDs depicted in FIGS. 24, 60, and 62-64 (e.g., SEQ ID NOs:140-143, 179-242, 315-584) and the sequence listing (e.g., SEQ ID NOs:585-802), comprises one or more amino acid substitutions in a framework region, and retains the binding and/or functional activity of the parent ICOS ABD.

In particular embodiments, the ICOS ABD comprises a variable light domain sequence with at least about 95%, about 96%, about 97%, about 98%, or about 99% sequence identity to a variable light domain sequence of a parent ICOS ABD (e.g., any one of the ICOS ABDs depicted in FIGS. 24, 60, and 62-64 (e.g., SEQ ID NOs:140-143, 179-242, 315-584) and the sequence listing (e.g., SEQ ID NOs:585-802)), comprises one or more conservative amino acid substitutions in a framework region, and retains the binding and/or functional activity of the parent ICOS ABD. In still further embodiments, the ICOS ABD comprises the variable light domain sequence of a parent ICOS ABD (e.g., any one of the ICOS ABDs depicted in FIGS. 24, 60, and 62-64 (e.g., SEQ ID NOs:140-143, 179-242, 315-584) and the sequence listing (e.g., SEQ ID NOs:585-802)) and has one or more amino acid substitutions, e.g., 1, 2, 3, 4, 5, 1-2, 1-3, 1-4 or 1-5 amino acid substitutions in the variable light domain sequence. In yet further embodiments, the amino acid substitutions fall within one or more framework regions.

Binding of an ICOS ABD to ICOS (e.g., human ICOS) can be measured by any suitable technique known in the art. In some embodiments, binding is measured by at least one of a Biacore, surface plasmon resonance (SPR) and/or BLI (biolayer interferometry, e.g., Octet assay) assay. In particular embodiments, the ICOS ABD is capable of binding human ICOS.

C. Fc Domains

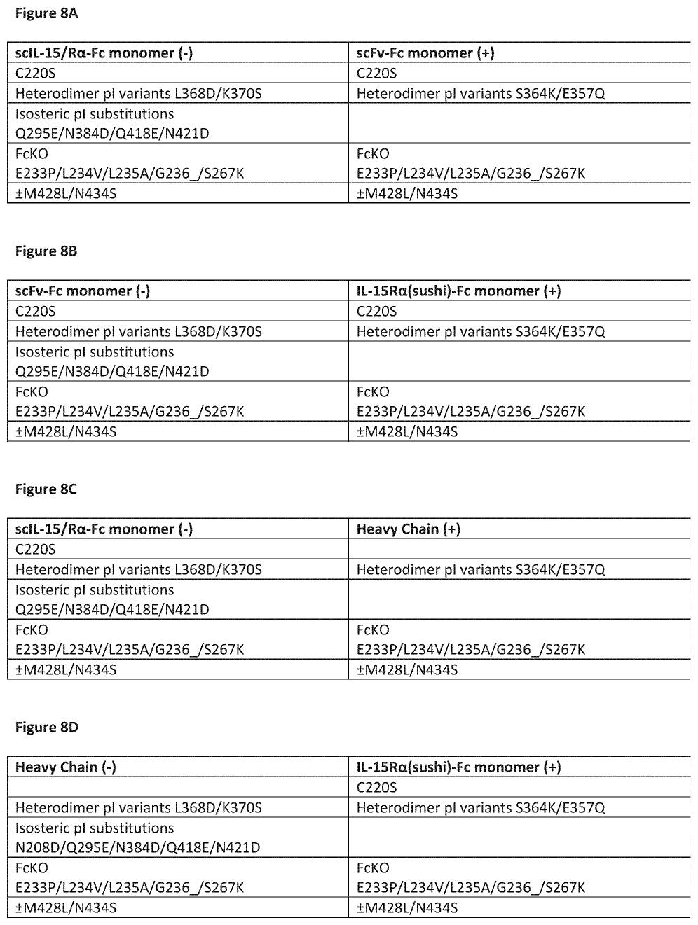

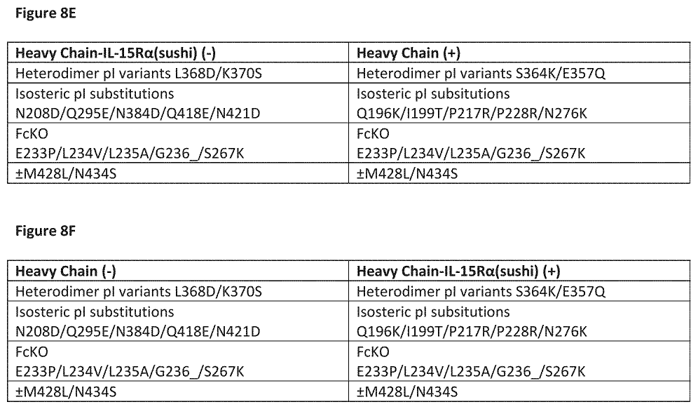

The Fc domain component of the invention is as described herein, which generally contains skew variants and/or optional pI variants and/or ablation variants are outlined herein. See for example the disclosure of WO2017/218707 under the heading “IV Heterodimeric Antibodies”, including sections IV.A, IV.B, IV.C, IV.D, IV.E, IV.F, IV.G, IV.H and IV.I, all of which are expressly incorporated by reference in their entirety. Of particular use in the heterodimeric fusion proteins of the present invention are Fc domains containing “skew variants”, “pI variants”, “ablation variants” and FcRn variants as outlined therein. Particularly useful Fc domains are those shown in FIG. 8. Thus, variant Fc domains derived from IgG1 can be used, as well as IgG4 variants with a S228P variant.

The Fc domains can be derived from IgG Fc domains, e.g., IgG1, IgG2, IgG3 or IgG4 Fc domains, with IgG1 Fc domains finding particular use in the invention. The following describes Fc domains that are useful for IL-15/IL-15Rα Fc fusion monomers and checkpoint antibody fragments of the targeted IL-15/IL-15Rα heterodimer proteins of the present invention.

Thus, the “Fc domain” includes the —CH2-CH3 domain, and optionally a hinge domain, and can be from human IgG1, IgG2, IgG3 or IgG4, with Fc domains derived from IgG1. In some of the embodiments herein, when a protein fragment, e.g., IL-15 or IL-15Rα is attached to an Fc domain, it is the C-terminus of the IL-15 or IL-15Rα construct that is attached to all or part of the hinge of the Fc domain; for example, it is generally attached to the sequence EPKS which is the beginning of the hinge. In other embodiments, when a protein fragment, e.g., IL-15 or IL-15Rα, is attached to an Fc domain, it is the C-terminus of the IL-15 or IL-15Rα construct that is attached to the CH1 domain of the Fc domain.

In some of the constructs and sequences outlined herein of an Fc domain protein, the C-terminus of the IL-15 or IL-15Rα protein fragment is attached to the N-terminus of a domain linker, the C-terminus of which is attached to the N-terminus of a constant Fc domain (N-IL-15 or IL-15Rα protein fragment-linker-Fc domain-C) although that can be switched (N-Fc domain-linker-IL-15 or IL-15Rα protein fragment-C). In other constructs and sequence outlined herein, C-terminus of a first protein fragment is attached to the N-terminus of a second protein fragment, optionally via a domain linker, the C-terminus of the second protein fragment is attached to the N-terminus of a constant Fc domain, optionally via a domain linker. In yet other constructs and sequences outlined herein, a constant Fc domain that is not attached to a first protein fragment or a second protein fragment is provided. A heterodimer Fc fusion protein can contain two or more of the exemplary monomeric Fc domain proteins described herein.

In some embodiments, the linker is a “domain linker”, used to link any two domains as outlined herein together, some of which are depicted in FIG. 9. While any suitable linker can be used, many embodiments utilize a glycine-serine polymer, including for example (GS)n, (GSGGS)n, (GGGGS)n, (GGGS)n, (GA)n, (GGGGA)n and (GGGA)n, where n is an integer of at least one (and generally from 1 to 2 to 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers.

In one embodiment, heterodimeric Fc fusion proteins contain at least two constant domains which can be engineered to produce heterodimers, such as pI engineering. Other Fc domains that can be used include fragments that contain one or more of the CH1, CH2, CH3, and hinge domains of the invention that have been pI engineered. In particular, the formats depicted in FIG. 14 and FIG. 32 are heterodimeric Fc fusion proteins, meaning that the protein has two associated Fc sequences self-assembled into a heterodimeric Fc domain and at least one fusion protein (e.g., 1, 2 or more fusion proteins) as more fully described below. In some cases, a first fusion protein is linked to a first Fc sequence and a second fusion protein is linked to a second Fc sequence. In other cases, a first fusion protein is linked to a first Fc sequence, and the first fusion protein is non-covalently attached to a second fusion protein that is not linked to an Fc sequence. In some cases, the heterodimeric Fc fusion protein contains a first fusion protein linked to a second fusion protein which is linked a first Fc sequence, and a second Fc sequence that is not linked to either the first or second fusion proteins.

Accordingly, in some embodiments the present invention provides heterodimeric Fc fusion proteins that rely on the use of two different heavy chain variant Fc sequences, that will self-assemble to form a heterodimeric Fc domain fusion polypeptide.

The present invention is directed to novel constructs to provide heterodimeric Fc fusion proteins that allow binding to one or more binding partners, ligands or receptors. The heterodimeric Fc fusion constructs are based on the self-assembling nature of the two Fc domains of the heavy chains of antibodies, e.g., two “monomers” that assemble into a “dimer”. Heterodimeric Fc fusions are made by altering the amino acid sequence of each monomer as more fully discussed below. Thus, the present invention is generally directed to the creation of heterodimeric Fc fusion proteins which can co-engage binding partner(s) or ligand(s) or receptor(s) in several ways, relying on amino acid variants in the constant regions that are different on each chain to promote heterodimeric formation and/or allow for ease of purification of heterodimers over the homodimers.

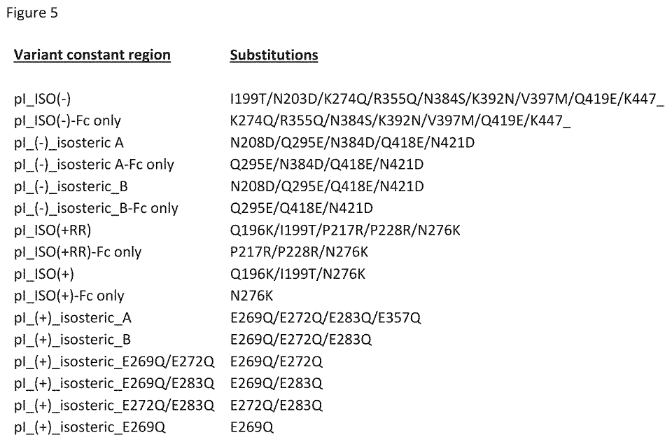

There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”, a number of which are shown in FIG. 4A-FIG. 4E. As discussed below, heterodimerization variants can include steric variants (e.g. the “knobs and holes” or “skew” variants described below and the “charge pairs” variants described below) as well as “pI variants”, which allows purification of homodimers away from heterodimers, as depicted in FIG. 5. As is generally described in WO2014/145806, hereby incorporated by reference in its entirety and specifically as below for the discussion of “heterodimerization variants”, useful mechanisms for heterodimerization include “knobs and holes” (“KIH”; sometimes herein as “skew” variants (see discussion in WO2014/145806), “electrostatic steering” or “charge pairs” as described in WO2014/145806, pI variants as described in WO2014/145806, and general additional Fc variants as outlined in WO2014/145806 and below.

In the present invention, there are several basic mechanisms that can lead to ease of purifying heterodimeric fusion proteins; one relies on the use of pI variants, such that each monomer has a different pI, thus allowing the isoelectric purification of A-A, A-B and B-B dimeric proteins. Alternatively, some formats also allow separation on the basis of size. As is further outlined below, it is also possible to “skew” the formation of heterodimers over homodimers. Thus, a combination of steric heterodimerization variants and pI or charge pair variants find particular use in the invention.

In general, embodiments of particular use in the present invention rely on sets of variants that include skew variants, that encourage heterodimerization formation over homodimerization formation, coupled with pI variants, which increase the pI difference between the two monomers.

Additionally, as more fully outlined below, depending on the format of the heterodimer Fc fusion protein, pI variants can be either contained within the constant and/or Fc domains of a monomer, or domain linkers can be used. That is, the invention provides pI variants that are on one or both of the monomers, and/or charged domain linkers as well. In addition, additional amino acid engineering for alternative functionalities may also confer pI changes, such as Fc, FcRn and KO variants.

In the present invention that utilizes pI as a separation mechanism to allow the purification of heterodimeric fusion proteins, amino acid variants can be introduced into one or both of the monomer polypeptides; that is, the pI of one of the monomers (referred to herein for simplicity as “monomer A”) can be engineered away from monomer B, or both monomer A and B change be changed, with the pI of monomer A increasing and the pI of monomer B decreasing. As discussed, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (e.g. aspartic acid to lysine) or changing a charged residue to a neutral residue (e.g., loss of a charge; lysine to serine). A number of these variants are shown in the Figures (see FIG. 5).

Accordingly, this embodiment of the present invention provides for creating a sufficient change in pI in at least one of the monomers such that heterodimers can be separated from homodimers. As will be appreciated by those in the art, and as discussed further below, this can be done by using a “wild type” heavy chain constant region and a variant region that has been engineered to either increase or decrease its pI (wt A−+B or wt A−−B), or by increasing one region and decreasing the other region (A+ −B− or A− B+).

Thus, in general, a component of some embodiments of the present invention are amino acid variants in the constant regions that are directed to altering the isoelectric point (pI) of at least one, if not both, of the monomers of a dimeric protein by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components. As is known in the art, different Fcs will have different starting pIs which are exploited in the present invention. In general, as outlined herein, the pIs are engineered to result in a total pI difference of each monomer of at least about 0.1 logs, with 0.2 to 0.5 being preferred as outlined herein.

As will be appreciated by those in the art, the number of pI variants to be included on each or both monomer(s) to get good separation will depend in part on the starting pI of the components. That is, to determine which monomer to engineer or in which “direction” (e.g., more positive or more negative), the sequences of the Fc domains, and in some cases, the protein domain(s) linked to the Fc domain are calculated and a decision is made from there. As is known in the art, different Fc domains and/or protein domains will have different starting pIs which are exploited in the present invention. In general, as outlined herein, the pIs are engineered to result in a total pI difference of each monomer of at least about 0.1 logs, with 0.2 to 0.5 being preferred as outlined herein.

Furthermore, as will be appreciated by those in the art and outlined herein, in some embodiments, heterodimers can be separated from homodimers on the basis of size. As shown in the Figures, for example, several of the formats allow separation of heterodimers and homodimers on the basis of size.

In the case where pI variants are used to achieve heterodimerization, by using the constant region(s) of Fc domains(s), a more modular approach to designing and purifying heterodimeric Fc fusion proteins is provided. Thus, in some embodiments, heterodimerization variants (including skew and purification heterodimerization variants) must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g. the minimization or avoidance of non-human residues at any particular position.

In addition, it should be noted that the pI variants of the heterodimerization variants give an additional benefit for the analytics and quality control process of Fc fusion proteins, as the ability to either eliminate, minimize and distinguish when homodimers are present is significant. Similarly, the ability to reliably test the reproducibility of the heterodimeric Fc fusion protein production is important.

1. Heterodimerization Variants

The present invention provides heterodimeric fusion proteins, including heterodimeric Fc fusion proteins in a variety of formats, which utilize heterodimeric variants to allow for heterodimeric formation and/or purification away from homodimers. The heterodimeric fusion constructs are based on the self-assembling nature of the two Fc domains, e.g., two “monomers” that assemble into a “dimer”.

There are a number of suitable pairs of sets of heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25 homodimer A/A:50% heterodimer A/B:25% homodimer B/B).

2. Steric Variants

In some embodiments, the formation of heterodimers can be facilitated by the addition of steric variants. That is, by changing amino acids in each heavy chain, different heavy chains are more likely to associate to form the heterodimeric structure than to form homodimers with the same Fc amino acid sequences. Suitable steric variants are included in in the FIG. 29 of U.S. Ser. No. 15/141,350, all of which is hereby incorporated by reference in its entirety, as well as in FIG. 4A-FIG. 4E.

One mechanism is generally referred to in the art as “knobs and holes”, referring to amino acid engineering that creates steric influences to favor heterodimeric formation and disfavor homodimeric formation can also optionally be used; this is sometimes referred to as “knobs and holes”, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g., these are “monomer corresponding sets) and C220E/P228E/368E paired with C220R/E224R/P228R/K409R.

Additional monomer A and monomer B variants that can be combined with other variants, optionally and independently in any amount, such as pI variants outlined herein or other steric variants that are shown in FIG. 37 of US 2012/0149876, all of which are incorporated expressly by reference herein.

In some embodiments, the steric variants outlined herein can be optionally and independently incorporated with any pI variant (or other variants such as Fc variants, FcRn variants, etc.) into one or both monomers, and can be independently and optionally included or excluded from the proteins of the invention.

A list of suitable skew variants is found in FIG. 4A-FIG. 4E. Of particular use in many embodiments are the pairs of sets including, but not limited to, S364K/E357Q:L368D/K370S; L368D/K370S:S364K; L368E/K370S:S364K; T411E/K360E/Q362E:D401K; L368D/K370S:S364K/E357L, K370S:S364K/E357Q and T366S/L368A/Y407V:T366W (optionally including a bridging disulfide, T366S/L368A/Y407V/Y349C:T366W/S354C or T366S/L368A/Y407V/S354C:T366W:Y349C). In terms of nomenclature, the pair “S364K/E357Q:L368D/K370S” means that one of the monomers has the double variant set S364K/E357Q and the other has the double variant set L368D/K370S; as above, the “strandedness” of these pairs depends on the starting pI.

3. pI (Isoelectric Point) Variants for Heterodimers

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Preferred combinations of pI variants are shown in FIG. 30 of U.S. Ser. No. 15/141,350, all of which are herein incorporated by reference in its entirety. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer comprising 208D/295E/384D/418E/421D variants (N208D/Q295E/N384D/Q418E/N421D when relative to human IgG1). In some instances, the second monomer comprises a positively charged domain linker, including (GKPGS)₄, particularly when scFv constructs are used. In some cases, the first monomer includes a CH1 domain, including position 208. Accordingly, in constructs that do not include a CH1 domain (for example for heterodimeric Fc fusion proteins that do not utilize a CH1 domain on one of the domains), a preferred negative pI variant Fc set includes 295E/384D/418E/421D variants (Q295E/N384D/Q418E/N421D when relative to human IgG1).

In some embodiments, mutations are made in the hinge domain of the Fc domain, including positions 221, 222, 223, 224, 225, 233, 234, 235 and 236. It should be noted that changes in 233-236 can be made to increase effector function (along with 327A) in the IgG2 backbone. Thus, pI mutations and particularly substitutions can be made in one or more of positions 221-225, with 1, 2, 3, 4 or 5 mutations finding use in the present invention. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 274, 296, 300, 309, 320, 322, 326, 327, 334 and 339. Again, all possible combinations of these 10 positions can be made; e.g., a pI antibody may have 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the mutations can be independently and optionally selected from position 355, 359, 362, 384, 389,392, 397, 418, 419, 444 and 447. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447. Exemplary embodiments of pI variants are provided in FIG. 5.

4. Isotypic Variants

In addition, many embodiments of the invention rely on the “importation” of pI amino acids at particular positions from one IgG isotype into another, thus reducing or eliminating the possibility of unwanted immunogenicity being introduced into the variants. A number of these are shown in FIG. 21 of US Publ. App. No. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant Fc fusion protein. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

5. Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US2014/0370013. As discussed herein, which monomer to engineer is generally decided by the inherent pI of each monomer.

6. Additional Fc Variants for Additional Functionality

In addition to pI amino acid variants, there are a number of useful Fc amino acid modification that can be made for a variety of reasons, including, but not limited to, altering binding to one or more FcγR receptors, altered binding to FcRn receptors, etc.

Accordingly, the proteins of the invention can include amino acid modifications, including the heterodimerization variants outlined herein, which includes the pI variants and steric variants. Each set of variants can be independently and optionally included or excluded from any particular heterodimeric fusion protein.

a. FcγR Variants

Accordingly, there are a number of useful Fc substitutions that can be made to alter binding to one or more of the FcγR receptors. Substitutions that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41), Ser. Nos. 11/174,287, 11/396,495, 11/538,406, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330L, 239D, 332E/330L, 243A, 243L, 264A, 264V and 299T.

In addition, amino acid substitutions that increase affinity for FcγRIIc can also be included in the Fc domain variants outlined herein. The substitutions described in, for example, U.S. Ser. Nos. 11/124,620 and 14/578,305 are useful.

In addition, there are additional Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 428L/434A, 259I/308F, 436I/428L, 436I or V/434S, 436V/428L, 259I/308F/428L, M252Y/S254T/T256E, H433K/N434F and L309D/Q311H/N434S.

b. Ablation Variants

Similarly, another category of functional variants includes “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific immunomodulatory antibodies desirable to ablate FcγRIIIa binding to eliminate or significantly reduce ADCC activity such that one of the Fc domains comprises one or more Fcγ receptor ablation variants. These ablation variants are depicted in FIG. 31 of U.S. Ser. No. 15/141,350, all of which are herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. It should be noted that the ablation variants referenced herein ablate FcγR binding but generally not FcRn binding.

Exemplary embodiments of ablation variants are provided in FIG. 5.

c. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

In addition, a monomeric Fc domain can comprise a set of amino acid substitutions that includes C220S/S267K/L368D/K370S or C220S/S267K/S364K/E357Q.

In addition, the heterodimeric Fc fusion proteins can comprise skew variants (e.g., a set of amino acid substitutions as shown in FIGS. 1A-1C of U.S. Ser. No. 15/141,350, all of which are herein incorporated by reference in its entirety), with particularly useful skew variants being selected from the group consisting of S364K/E357Q:L368D/K370S; L368D/K370S:S364K; L368E/K370S:S364K; T411E/Q362E:D401K; L368D/K370S:S364K/E357L, K370S:S364K/E357Q, T366S/L368A/Y407V:T366W, T366S/L368A/Y407V/S354C:T366W/Y349C and T366S/L368A/Y407V/Y349C:T366W/S354C, optionally ablation variants, optionally charged domain linkers and the heavy chain comprises pI variants.

In some embodiments, the Fc domain comprising an amino acid substitution selected from the group consisting of: 236R, 239D, 239E, 243L, M252Y, V259I, 267D, 267E, 298A, V308F, 328F, 328R, 330L, 332D, 332E, M428L, N434A, N434S, 236R/328R, 239D/332E, M428L, 236R/328F, V259I/V308F, 267E/328F, M428L/N434S, Y436I/M428L, Y436V/M428L, Y436I/N434S, Y436V/N434S, 239D/332E/330L, M252Y/S254T/T256E, V259I/V308F/M428L, E233P/L234V/L235A/G236del/S267K, G236R/L328R and PVA/S267K. In some cases, the Fc domain comprises the amino acid substitution 239D/332E. In other cases, the Fc domain comprises the amino acid substitution G236R/L328R or PVA/S267K.

In one embodiment, a particular combination of skew and pI variants that finds use in the present invention is T366S/L368A/Y407V:T366W (optionally including a bridging disulfide, T366S/L368A/Y407V/Y349C:T366W/S354C or T366S/L368A/Y407V/S354C:T366W/Y349C) with one monomer comprises Q295E/N384D/Q418E/N481D and the other a positively charged domain linker. As will be appreciated in the art, the “knobs in holes” variants do not change pI, and thus can be used on either monomer.

### III. Useful Formats of ICOS-Targeted x IL-15/IL-15Rα Fc Fusion Proteins

Provided herein are heterodimeric Fc fusion proteins that can bind to the checkpoint inhibitor ICOS antigen and can complex with the common gamma chain (γc; CD132) and/or the IL-2 receptor β-chain (IL-2Rβ; CD122). In some embodiments, the heterodimeric Fc fusion proteins contain an IL-15/IL-15Rα-Fc fusion protein and an antibody fusion protein. The IL-15/IL-15Rα-Fc fusion protein can include as IL-15 protein (generally including amino acid substitutions) covalently attached to an IL-15Rα, and an Fc domain. Optionally, the IL-15 protein and IL-15Rα protein are noncovalently attached.

As shown in FIG. 25A-FIG. 25CC, there are a number of useful formats of the targeted IL-15/IL-15Rα heterodimeric fusion proteins of the invention. In general, the heterodimeric fusion proteins of the invention have three functional components: an IL-15/IL-15Rα(sushi) component, an anti-ICOS component, and an Fc component, each of which can take different forms as outlined herein and each of which can be combined with the other components in any configuration. In some embodiments, the anti-ICOS component includes any of the ICOS binding domains provided herein. Suitable human ICOS antigen binding domains for use in the ICOS-targeted x IL-15/IL-15Rα Fc fusion proteins include, but are not limited to, those listed in FIGS. 24, 60, and 62-64 (e.g., SEQ ID NOs:140-143, 179-242, 315-584) and the sequence listing (e.g., SEQ ID NOs:585-802).

The first and the second Fc domains can have a set of amino acid substitutions selected from the group consisting of a) S267K/L368D/K370S:S267K/S364K/E357Q; b) S364K/E357Q:L368D/K370S; c) L368D/K370S:S364K; d) L368E/K370S:S364K; e) T411E/K360E/Q362E:D401K; f) L368D/K370S:S364K/E357L and g) K370S:S364K/E357Q, according to EU numbering.

In some embodiments, the first and/or the second Fc domains have an additional set of amino acid substitutions comprising Q295E/N384D/Q418E/N421D, according to EU numbering.

Optionally, the first and/or the second Fc domains have an additional set of amino acid substitutions consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to EU numbering.

Optionally, the first and/or second Fc domains have 428L/434S variants (e.g., M428L/N434S variants) for half-life extension.

A. scIL15Rα-IL15-Fc x scFv-Fc

One embodiment is shown in FIG. 25A, and comprises two monomers. The first monomer comprises, from N- to C-terminus, the IL-15Rα(sushi) domain-(domain linker)-IL-15 variant-(domain linker)-CH2-CH3 (with the second domain linker frequently being a hinge domain); and the second monomer comprises VH-scFv linker-VL-hinge-CH2-CH3 or VL-scFv linker-VH-hinge-CH2-CH3, although in either orientation a domain linker can be substituted for the hinge. This is generally referred to as “scIL15Rα-IL15-Fc x scFv-Fc”, with the “sc” standing for “single chain” referring to the attachment of the IL-15 variant and IL-15Rα(sushi) domain using a covalent linker.

In the scIL15Rα-IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the scIL15Rα-IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the scIL15Rα-IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60A, with either the IL-15 N4D/N65D variant or the IL-15 D30N/N65D variant or the IL-15 D30N/E64Q/N65D variant.

In the scIL15Rα-IL15-Fc x scFv-Fc format, one preferred embodiment utilizes the skew variant pair S364K/E357Q:L368D/K370S.

In the scIL15Rα-IL15-Fc x scFv-Fc format, one preferred embodiment utilizes the ICOS ABD having the variable heavy and light domain pair 2A5B4_H1L1 or the variable heavy and light domain pair 4.1D3.Q1E_H0L0 as shown in FIG. 12 and the skew variant pair S364K/E357Q:L368D/K370S.

In the scIL15Rα-IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60. and the skew variant pair S364K/E357Q:L368D/K370S with either the IL-15 N4D/N65D variant or the IL-15 D30N/N65D variant or the IL-15 D30N/E64Q/N65D variant.

In the scIL15Rα-IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

B. scIL15-IL15Rα-Fc x scFv-Fc

One embodiment is shown in FIG. 25B, and comprises two monomers. The first monomer comprises, from N- to C-terminus, the IL-15 variant-(domain linker)-IL-15Rα(sushi) domain-(domain linker)-CH2-CH3 (with the second domain linker frequently being a hinge domain), and the second monomer comprises VH-scFv linker-VL-hinge-CH2-CH3 or VL-scFv linker-VH-hinge-CH2-CH3, although in either orientation a domain linker can be substituted for the hinge. This is generally referred to as “scIL15-IL15Rα-Fc x scFv-Fc”, with the “sc” standing for “single chain” referring to the attachment of the IL-15 variant and IL-15Rα(sushi) domain using a covalent linker.

In the scIL15-IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the scIL15-IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the scIL15-IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60A, with either the IL-15 N4D/N65D variant or the IL-15 D30N/N65D variant or the IL-15 D30N/E64Q/N65D variant.

In the scIL15-IL15Rα-Fc x scFv-Fc format, one preferred embodiment utilizes the skew variant pair S364K/E357Q:L368D/K370S.

In the scIL15-IL15Rα-Fc x scFv-Fc format, one preferred embodiment utilizes the ICOS ABD having the variable heavy and light domain pair 2A5B4_H1L1 or the variable heavy and light domain pair 4.1D3.Q1E_H0L0 as shown in FIG. 12 and the skew variant pair S364K/E357Q:L368D/K370S.

In the scIL15-IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60. and the skew variant pair S364K/E357Q:L368D/K370S with either the IL-15 N4D/N65D variant or the IL-15 D30N/N65D variant or the IL-15 D30N/E64Q/N65D variant.

In the scIL15-IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

C. IL15-Fc x scFv-Fc

One embodiment is shown in FIG. 25C, and comprises two monomers. The first monomer comprises, from N- to C-terminus, the IL-15 variant-(domain linker)-CH2-CH3 (with the second domain linker frequently being a hinge domain), and the second monomer comprises VH-scFv linker-VL-hinge-CH2-CH3 or VL-scFv linker-VH-hinge-CH2-CH3, although in either orientation a domain linker can be substituted for the hinge. This is generally referred to as “IL15-Fc x scFv-Fc”.

In the IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60A, with either the IL-15 N4D/N65D variant or the IL-15 D30N/N65D variant or the IL-15 D30N/E64Q/N65D variant.

In the IL15-Fc x scFv-Fc format, one preferred embodiment utilizes the skew variant pair S364K/E357Q:L368D/K370S.

In the IL15-Fc x scFv-Fc format, one preferred embodiment utilizes the ICOS ABD having the variable heavy and light domain pair 2A5B4_H1L1 or the variable heavy and light domain pair 4.1D3.Q1E_H0L0 as shown in FIG. 12 and the skew variant pair S364K/E357Q:L368D/K370S.

In the IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60. and the skew variant pair S364K/E357Q:L368D/K370S with either the IL-15 N4D/N65D variant or the IL-15 D30N/N65D variant or the IL-15 D30N/E64Q/N65D variant.

In the IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

D. ncIL15+IL15Rα-Fc x scFv-Fc

This embodiment is shown in FIG. 25D, and comprises three monomers. The first monomer comprises, from N- to C-terminus, the IL-15Rα(sushi) domain-domain linker-CH2-CH3, and the second monomer comprises vh-scFv linker-vl-hinge-CH2-CH3 or vl-scFv linker-vh-hinge-CH2-CH3, although in either orientation a domain linker can be substituted for the hinge. The third monomer is the variant IL-15 domain. This is generally referred to as “ncIL15+IL15Rα-Fc x scFv-Fc” or “scFv-Fc x ncIL15+IL15Rα-Fc” with the “nc” standing for “non-covalent” referring to the self-assembling non-covalent attachment of the IL-15 variant and IL-15Rα(sushi) domain.

In the ncIL15+IL15Rα-Fc x scFv-Fc format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15+IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15+IL15Rα-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15+IL15Rα-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In the ncIL15+IL15Rα-Fc x scFv-Fc format, one preferred embodiment utilizes the skew variant pair S364K/E357Q:L368D/K370S.

In the ncIL15+IL15Rα-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S.

In the ncIL15+IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the ncIL15+IL15Rα-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer side), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

E. ncIL15Rα+IL15-Fc x scFv-Fc

This embodiment is shown in FIG. 25E, and comprises three monomers. The first monomer comprises, from N- to C-terminus, a variant IL15-domain linker-CH2-CH3, and the second monomer comprises vh-scFv linker-vl-hinge-CH2-CH3 or vl-scFv linker-vh-hinge-CH2-CH3, although in either orientation a domain linker can be substituted for the hinge. The third monomer is the IL-15Rα(sushi) domain. This is generally referred to as “ncIL15Rα+IL15-Fc x scFv-Fc” or “scFv-Fc x ncIL15Rα+IL15-Fc” with the “nc” standing for “non-covalent” referring to the self-assembling non-covalent attachment of the IL-15 variant and IL-15Rα(sushi) domain.

In the ncIL15Rα+IL15-Fc x scFv-Fc format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15Rα+IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15Rα+IL15-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the ncIL15Rα+IL15-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In the ncIL15Rα+IL15-Fc x scFv-Fc format, one preferred embodiment utilizes the skew variant pair S364K/E357Q:L368D/K370S.

In the ncIL15Rα+IL15-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S.

In the ncIL15Rα+IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the ncIL15Rα+IL15-Fc x scFv-Fc format, one preferred embodiment utilizes the anti-ICOSABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

F. dsIL15+IL15Rα-Fc x scFv-Fc

This embodiment is shown in FIG. 25F and comprises three monomers. The first monomer comprises, from N- to C-terminus, a variant IL-15Rα(sushi) domain-domain linker-CH2-CH3, wherein the variant IL-15Rα(sushi) domain has an engineered cysteine residue and the second monomer comprises vh-scFv linker-vl-hinge-CH2-CH3 or vl-scFv linker-vh-hinge-CH2-CH3, although in either orientation a domain linker can be substituted for the hinge. The third monomer is the variant IL-15, also engineered to have a cysteine variant amino acid, thus allowing a disulfide bridge to form between the IL-15Rα(sushi) domain and the variant IL-15 domain. This is generally referred to as “dsIL15+IL15Rα-Fc x scFv-Fc” or “scFv-Fc x dsIL15+IL15Rα-Fc”, with the “ds” standing for “disulfide”.

In the dsIL15+IL15Rα-Fc x scFv-Fc format, one preferred embodiment utilizes the ICOS ABD having any of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the dsIL15+IL15Rα-Fc x scFv-Fc format, one preferred embodiment utilizes the ICOS ABD having the variable heavy and light domain pair described herein with either the IL-15 N4D/N65D variant or the IL-15 D30N/N65D variant or the IL-15 D30N/E64Q/N65D variant, as well as appropriate cysteine substitutions.

In the dsIL15+IL15Rα-Fc x scFv-Fc format, one preferred embodiment utilizes the skew variant pair S364K/E357Q:L368D/K370S.

In the dsIL15+IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S.

In the dsIL15+IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 with either the IL-15 N4D/N65D variant or the IL-15 D30N/N65D variant or the IL-15 D30N/E64Q/N65D variant with the appropriate cysteine substitutions.

In the dsIL15+IL15Rα-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both monomers, and optionally the 428L/434S variants on both sides.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

G. dsIL15Rα+IL15-Fc x scFv-Fc

This embodiment is shown in FIG. 25G and comprises three monomers. The first monomer comprises, from N- to C-terminus, a variant IL-15-domain linker-CH2-CH3, wherein the variant IL-15 has an engineered cysteine residue and the second monomer comprises vh-scFv linker-vl-hinge-CH2-CH3 or vl-scFv linker-vh-hinge-CH2-CH3, although in either orientation a domain linker can be substituted for the hinge. The third monomer is a variant IL-15Rα(sushi) domain, also engineered to have a cysteine variant amino acid, thus allowing a disulfide bridge to form between the IL-15Rα(sushi) domain and the variant IL-15. This is generally referred to as “dsIL15Rα+IL15-Fc x scFv-Fc” or “scFv-Fc x dsIL15Rα+IL15-Fc”, with the “ds” standing for “disulfide”.

In the dsIL15Rα+IL15-Fc x scFv-Fc format, one preferred embodiment utilizes the ICOS ABD having any of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the dsIL15Rα+IL15-Fc x scFv-Fc format, one preferred embodiment utilizes the ICOS ABD having the variable heavy and light domain pair described herein with either the IL-15 N4D/N65D variant or the IL-15 D30N/N65D variant or the IL-15 D30N/E64Q/N65D variant, as well as appropriate cysteine substitutions.

In the dsIL15Rα+IL15-Fc x scFv-Fc format, one preferred embodiment utilizes the skew variant pair S364K/E357Q:L368D/K370S.

In the dsIL15Rα+IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S.

In the dsIL15Rα+IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 with either the IL-15 N4D/N65D variant or the IL-15 D30N/N65D variant or the IL-15 D30N/E64Q/N65D variant with the appropriate cysteine substitutions.

In the dsIL15Rα+IL15-Fc x scFv-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

H. scIL15Rα-IL15-Fc x Fab-Fc

This embodiment is shown in FIG. 25H, and comprises three monomers. The first monomer comprises, from N- to C-terminus, the IL-15Rα(sushi) domain-domain linker-variant IL-15-domain linker-CH2-CH3 and the second monomer comprises a heavy chain, VH-CH1-hinge-CH2-CH3. The third monomer is a light chain, VL-CL. This is generally referred to as “scIL15Rα-IL15-Fc x Fab-Fc”, with the “sc” standing for “single chain”.

The “scIL15Rα-IL15-Fc x Fab-Fc” format (FIG. 25C) comprises IL-15Rα(sushi) fused to IL-15 by a variable length linker (termed “scIL-15/Rα”) which is then fused to the N-terminus of a heterodimeric Fc-region, with a variable heavy chain (VH) fused to the other side of the heterodimeric Fc, while a corresponding light chain is transfected separately so as to form a Fab with the VH. Preferred combinations of variants for this embodiment are found in FIG. 61A-FIG. 61P.

In the scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the scIL15Rα-IL15-Fc x Fab-Fc format, one preferred embodiment utilizes the skew variant pair S364K/E357Q:L368D/K370S.

In the scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the scIL15Rα-IL15-Fc x Fab-Fc format, one preferred embodiment utilizes the ICOS ABD having the variable heavy and light domain of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

I. scIL15-IL15Rα-Fc x Fab-Fc

This embodiment is shown in FIG. 25I, and comprises three monomers. The first monomer comprises, from N- to C-terminus, a variant IL-15-domain linker-IL-15Rα(sushi) domain-domain linker-CH2-CH3 and the second monomer comprises a heavy chain, VH-CH1-hinge-CH2-CH3. The third monomer is a light chain, VL-CL. This is generally referred to as “scIL15-IL15Rα-Fc x Fab-Fc”, with the “sc” standing for “single chain”.

In the scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the scIL15-IL15Rα-Fc x Fab-Fc format, one preferred embodiment utilizes the skew variant pair S364K/E357Q:L368D/K370S.

In the scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the scIL15-IL15Rα-Fc x Fab-Fc format, one preferred embodiment utilizes the ICOS ABD having the variable heavy and light domain of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

J. IL15-Fc x Fab-Fc

This embodiment is shown in FIG. 25J, and comprises three monomers. The first monomer comprises, from N- to C-terminus, a variant IL-15-domain linker-CH2-CH3 and the second monomer comprises a heavy chain, VH-CH1-hinge-CH2-CH3. The third monomer is a light chain, VL-CL. This is generally referred to as “IL15-Fc x Fab-Fc”.

In the IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the IL15-Fc x Fab-Fc format, one preferred embodiment utilizes the skew variant pair S364K/E357Q:L368D/K370S.

In the IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the IL15-Fc x Fab-Fc format, one preferred embodiment utilizes the ICOS ABD having the variable heavy and light domain of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

K. ncIL15+IL15Rα-Fc x Fab-Fc

This embodiment is shown in FIG. 25K, and comprises three monomers. The first monomer comprises, from N- to C-terminus, the IL-15Rα(sushi) domain-domain linker-CH2-CH3, and the second monomer comprises a heavy chain, VH-CH1-hinge-CH2-CH3. The third monomer is the variant IL-15 domain. This is generally referred to as “ncIL15+IL15Rα-Fc x Fab-Fc”, with the “nc” standing for “non-covalent” referring to the self-assembling non-covalent attachment of the IL-15 variant and IL-15Rα(sushi)domain. ICOS-targeted IL-15/Rα-Fc fusion proteins of this format are depicted in the Figures.

In the ncIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the ncIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the ncIL15+IL15Rα-Fc x Fab-Fc format, one preferred embodiment utilizes the skew variant pair S364K/E357Q:L368D/K370S.

In the ncIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the ncIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 with either the IL-15 N4D/N65D variant or the IL-15 D30N/N65D variant or the IL-15 D30N/E64Q/N65D variant.

In the ncIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on the a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

L. ncIL15Rα+IL15-Fc x Fab-Fc

This embodiment is shown in FIG. 25L, and comprises three monomers. The first monomer comprises, from N- to C-terminus, the variant IL-15-domain linker-CH2-CH3, and the second monomer comprises a heavy chain, VH-CH1-hinge-CH2-CH3. The third monomer is the IL-15Rα(sushi) domain. This is generally referred to as “ncIL15Rα+IL15-Fc x Fab-Fc”, with the “nc” standing for “non-covalent” referring to the self-assembling non-covalent attachment of the IL-15 variant and IL-15Rα(sushi)domain. ICOS-targeted IL-15/Rα-Fc fusion proteins of this format are depicted in the Figures.

In the ncIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the ncIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the ncIL15Rα+IL15-Fc x Fab-Fc format, one preferred embodiment utilizes the skew variant pair S364K/E357Q:L368D/K370S.

In the ncIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the ncIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 with either the IL-15 N4D/N65D variant or the IL-15 D30N/N65D variant or the IL-15 D30N/E64Q/N65D variant.

In the ncIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

M. dsIL15+IL15Rα-Fc x Fab-Fc

This embodiment is shown in FIG. 25M and comprises three monomers. The first monomer comprises, from N- to C-terminus, a variant IL-15Rα(sushi)domain-domain linker-CH2-CH3, wherein the variant IL-15Rα(sushi)domain has been engineered to contain a cysteine residue, and the second monomer comprises a heavy chain, VH-CH1-hinge-CH2-CH3. The third monomer is the variant IL-15 domain, also engineered to have a cysteine residue, such that a disulfide bridge is formed under native cellular conditions. This is generally referred to as “dsIL15+IL15Rα-Fc x Fab-Fc”, with the “ds” standing for “disulfide”.

In the dsIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the dsIL15+IL15Rα-Fc x Fab-Fc format, one preferred embodiment utilizes the ICOS ABD having the variable heavy and light domain pair described herein with either the IL-15 N4D/N65D variant or the IL-15 D30N/N65D variant or the IL-15 D30N/E64Q/N65D variant, with the appropriate cysteine amino acid substitutions.

In the dsIL15+IL15Rα-Fc x Fab-Fc format, one preferred embodiment utilizes the skew variant pair S364K/E357Q:L368D/K370S.

In the dsIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the dsIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant with appropriate cysteine substitutions.

In the dsIL15+IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

N. dsIL15Rα+IL15-Fc x Fab-Fc

This embodiment is shown in FIG. 25N and comprises three monomers. The first monomer comprises, from N- to C-terminus, a variant IL-15-domain linker-CH2-CH3, wherein the variant IL-15 has been engineered to contain a cysteine residue, and the second monomer comprises a heavy chain, VH-CH1-hinge-CH2-CH3. The third monomer is the variant IL-15Rα(sushi) domain, also engineered to have a cysteine residue, such that a disulfide bridge is formed under native cellular conditions. This is generally referred to as “dsIL15Rα+IL15-Fc x Fab-Fc”, with the “ds” standing for “disulfide”.

In the dsIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the dsIL15Rα+IL15-Fc x Fab-Fc format, one preferred embodiment utilizes the ICOS ABD having the variable heavy and light domain pair described herein with either the IL-15 N4D/N65D variant or the IL-15 D30N/N65D variant or the IL-15 D30N/E64Q/N65D variant, with the appropriate cysteine amino acid substitutions.

In the dsIL15Rα+IL15-Fc x Fab-Fc format, one preferred embodiment utilizes the skew variant pair S364K/E357Q:L368D/K370S.

In the dsIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the dsIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant with appropriate cysteine substitutions.

In the dsIL15Rα+IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on the heavy chain) and L368D/K370S (on the dsIL15Rα+IL15-Fc side), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both monomers, and optionally the 428L/434S variants on both sides.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

O. Fab-Fc-scIL15Rα-IL15 x Fab-Fc

This embodiment is shown in FIG. 25O, and comprises three monomers (although the fusion protein is a tetramer). The first monomer comprises a heavy chain, VH-CH1-hinge-CH2-CH3. The second monomer comprises a heavy chain with a C-terminal scIL15Rα-IL15 complex, VH-CH1-hinge-CH2-CH3-domain linker-IL-15Rα(sushi)domain-domain linker-IL-15 variant. The third (and fourth) monomer are light chains, VL-CL. This is generally referred to as “Fab-Fc-scIL15Rα-IL15 x Fab-Fc”, with the “sc” standing for “single chain”. This binds the ICOS molecule bivalently.

In the Fab-Fc-scIL15Rα-IL15 x Fab-Fc format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the Fab-Fc-scIL15Rα-IL15 x Fab-Fc format, one preferred embodiment utilizes the ICOS ABD having the variable heavy and light domain pair described herein, with either the IL-15 N4D/N65D variant or the IL-15 D30N/N65D variant or the IL-15 D30N/E64Q/N65D variant.

In the Fab-Fc-scIL15Rα-IL15 x Fab-Fc format, one preferred embodiment utilizes the skew variant pair S364K/E357Q:L368D/K370S.

In the Fab-Fc-scIL15Rα-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-scIL15Rα-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-scIL15Rα-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

P. Fab-Fc-scIL15-IL15Rα x Fab-Fc

This embodiment is shown in FIG. 25P, and comprises three monomers (although the fusion protein is a tetramer). The first monomer comprises a heavy chain, VH-CH1-hinge-CH2-CH3. The second monomer comprises a heavy chain with a C-terminal scIL15-IL15Rα complex, VH-CH1-hinge-CH2-CH3-domain linker-IL-15 variant-domain linker-IL-15Rα(sushi) domain. The third (and fourth) monomer are light chains, VL-CL. This is generally referred to as “Fab-Fc-scIL15-IL15Rα x Fab-Fc”, with the “sc” standing for “single chain”. This binds the ICOS molecule bivalently.

In the Fab-Fc-scIL15-IL15Rα x Fab-Fc format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the Fab-Fc-scIL15-IL15Rα x Fab-Fc format, one preferred embodiment utilizes the ICOS ABD having the variable heavy and light domain pair described herein, with either the IL-15 N4D/N65D variant or the IL-15 D30N/N65D variant or the IL-15 D30N/E64Q/N65D variant.

In the Fab-Fc-scIL15-IL15Rα x Fab-Fc format, one preferred embodiment utilizes the skew variant pair S364K/E357Q:L368D/K370S.

In the Fab-Fc-scIL15-IL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-scIL15-IL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-scIL15-IL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

Q. Fab-Fc-IL15 x Fab-Fc

This embodiment is shown in FIG. 25Q, and comprises three monomers (although the fusion protein is a tetramer). The first monomer comprises a heavy chain, VH-CH1-hinge-CH2-CH3. The second monomer comprises a heavy chain with a C-terminal IL15, VH-CH1-hinge-CH2-CH3-domain linker-IL-15 variant. The third (and fourth) monomer are light chains, VL-CL. This is generally referred to as “Fab-Fc-IL15 x Fab-Fc”. This binds the ICOS molecule bivalently. In another embodiment, the first monomer also comprises a C-terminal IL15, VH-CH1-hinge-CH2-CH3-domain linker-IL-15 variant, and is generally referred to as “Fab-Fc-IL15 x Fab-Fc IL15”.

In the Fab-Fc-IL15 x Fab-Fc format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the Fab-Fc-IL15 x Fab-Fc format, one preferred embodiment utilizes the ICOS ABD having the variable heavy and light domain pair described herein, with either the IL-15 N4D/N65D variant or the IL-15 D30N/N65D variant or the IL-15 D30N/E64Q/N65D variant.

In the Fab-Fc-IL15 x Fab-Fc format, one preferred embodiment utilizes the skew variant pair S364K/E357Q:L368D/K370S.

In the Fab-Fc-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-IL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

R. Fab-Fc-IL15Rα+ncIL15 x Fab-Fc

This embodiment is shown in FIG. 25R, and comprises four monomers (although the heterodimeric fusion protein is a pentamer). The first monomer comprises a heavy chain, VH-CH1-hinge-CH2-CH3. The second monomer comprises a heavy chain with a C-terminal IL-15Rα(sushi) domain: e.g., VH-CH1-hinge-CH2-CH3-domain linker-IL-15Rα(sushi) domain. The third monomer is a variant IL-15 domain. The fourth (and fifth) monomer are light chains, VL-CL. This is generally referred to as “Fab-Fc-IL15Rα+ncIL15 x Fab-Fc”, with the “nc” standing for “non-covalent”. This also binds the ICOS bivalently.

In the Fab-Fc-IL15Rα+ncIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15Rα+ncIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the Fab-Fc-IL15Rα+ncIL15 x Fab-Fc format, one preferred embodiment utilizes the skew variant pair S364K/E357Q:L368D/K370S.

In the Fab-Fc-IL15Rα+ncIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15Rα+ncIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-IL15Rα+ncIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

S. Fab-Fc-IL15+ncIL15Rα x Fab-Fc

This embodiment is shown in FIG. 25S, and comprises four monomers (although the heterodimeric fusion protein is a pentamer). The first monomer comprises a heavy chain, VH-CH1-hinge-CH2-CH3. The second monomer comprises a heavy chain with a C-terminal variant IL-15: e.g., VH-CH1-hinge-CH2-CH3-domain linker-IL-15. The third monomer is a IL-15Rα(sushi) domain. The fourth (and fifth) monomer are light chains, VL-CL. This is generally referred to as “Fab-Fc-IL15+ncIL15Rα x Fab-Fc”, with the “nc” standing for “non-covalent”. This also binds the ICOS bivalently.

In the Fab-Fc-IL15+ncIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15+ncIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the Fab-Fc-IL15+ncIL15Rα x Fab-Fc format, one preferred embodiment utilizes the skew variant pair S364K/E357Q:L368D/K370S.

In the Fab-Fc-IL15+ncIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15+ncIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-IL15+ncIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

T. Fab-Fc-IL15 x Fab-Fc-IL15Rα

This embodiment is shown in FIG. 25T, and comprises three monomers (although the fusion protein is a tetramer). The first monomer comprises a heavy chain with a C-terminal variant IL-15: e.g. VH-CH1-hinge-CH2-CH3-domain linker-IL-15. The second monomer comprises a heavy chain with a C-terminal IL15Rα(sushi) domain: e.g. VH-CH1-hinge-CH2-CH3-domain linker-IL-15Rα(sushi) domain. The third (and fourth) monomer are light chains, VL-CL. This is generally referred to as “Fab-Fc-IL15 x Fab-Fc-IL15Rα”. This binds the ICOS molecule bivalently.

In the Fab-Fc-IL15 x Fab-Fc-IL15Rα format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the Fab-Fc-IL15 x Fab-Fc-IL15Rα format, one preferred embodiment utilizes the ICOS ABD having the variable heavy and light domain pair described herein, with either the IL-15 N4D/N65D variant or the IL-15 D30N/N65D variant or the IL-15 D30N/E64Q/N65D variant.

In the Fab-Fc-IL15 x Fab-Fc-IL15Rα format, one preferred embodiment utilizes the skew variant pair S364K/E357Q:L368D/K370S.

In the Fab-Fc-IL15 x Fab-Fc-IL15Rα format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15 x Fab-Fc-IL15Rα format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-IL15 x Fab-Fc-IL15Rα format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

U. Fab-Fc-IL15Rα+dsIL15 x Fab-Fc

This embodiment is shown in FIG. 25U and comprises four monomers (although the heterodimeric fusion protein is a pentamer). The first monomer comprises a heavy chain, VH-CH1-hinge-CH2-CH3. The second monomer comprises a heavy chain with a C-terminal variant IL-15Rα(sushi) domain: e.g., VH-CH1-hinge-CH2-CH3-domain linker-IL-15Rα(sushi) domain, where the IL-15Rα(sushi) domain has been engineered to contain a cysteine residue. The third monomer is a variant IL-15 domain, which has been engineered to contain a cysteine residue, such that the IL-15 complex is formed under physiological conditions. The fourth (and fifth) monomer are light chains, VL-CL. This is generally referred to as “Fab-Fc-IL15Rα+dsIL15 x Fab-Fc”, with the “ds” standing for “disulfide”, and it binds ICOS bivalently.

In the Fab-Fc-IL15Rα+dsIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15Rα+dsIL15 x Fab-Fc format, one preferred embodiment utilizes the ICOS ABD having the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant, with the appropriate cysteine amino acid substitutions.

In the Fab-Fc-IL15Rα+dsIL15 x Fab-Fc format, one preferred embodiment utilizes the skew variant pair S364K/E357Q:L368D/K370S.

In the Fab-Fc-IL15Rα+dsIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15Rα+dsIL15 x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant with appropriate cysteine substitutions.

In the Fab-Fc-IL15Rα+dsIL15 x Fab-Fc format, one preferred embodiment utilizes the ICOS ABD the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

V. Fab-Fc-IL15+dsIL15Rα x Fab-Fc

This embodiment is shown in FIG. 25V and comprises four monomers (although the heterodimeric fusion protein is a pentamer). The first monomer comprises a heavy chain, VH-CH1-hinge-CH2-CH3. The second monomer comprises a heavy chain with a C-terminal variant IL-15 domain: e.g., VH-CH1-hinge-CH2-CH3-domain linker-IL-15 domain, where the variant IL-15 domain has been engineered to contain a cysteine residue. The third monomer is a variant IL-15Rα(sushi) domain, which has been engineered to contain a cysteine residue, such that the IL-15 complex is formed under physiological conditions. The fourth (and fifth) monomer are light chains, VL-CL. This is generally referred to as “Fab-Fc-IL15+dsIL15Rα x Fab-Fc”, with the “ds” standing for “disulfide”, and it binds ICOS bivalently.

In the Fab-Fc-IL15+dsIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15+dsIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant, with the appropriate cysteine amino acid substitutions.

In the Fab-Fc-IL15+dsIL15Rα x Fab-Fc format, one preferred embodiment utilizes the skew variant pair S364K/E357Q:L368D/K370S.

In the Fab-Fc-IL15+dsIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15+dsIL15Rα x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant with appropriate cysteine substitutions.

In the Fab-Fc-IL15+dsIL15Rα x Fab-Fc format, one preferred embodiment utilizes the ICOS ABD the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

W. Fab-Fc-IL15 x Fab-Fc-IL15Rα w/ ds

This embodiment is shown in FIG. 25W, and comprises three monomers (although the fusion protein is a tetramer). The first monomer comprises a heavy chain with a C-terminal variant IL-15: e.g. VH-CH1-hinge-CH2-CH3-domain linker-IL-15, where the variant IL-15 domain has been engineered to contain a cysteine residue. The second monomer comprises a heavy chain with a C-terminal variant IL15Rα(sushi) domain: e.g. VH-CH1-hinge-CH2-CH3-domain linker-IL-15Rα(sushi) domain, which has been engineered to contain a cysteine residue, such that the IL-15 complex is formed under physiological conditions. The third (and fourth) monomer are light chains, VL-CL. This is generally referred to as “Fab-Fc-IL15 x Fab-Fc-IL15Rα w/ ds”. This binds the ICOS molecule bivalently.

In the Fab-Fc-IL15 x Fab-Fc-IL15Rα w/ ds format, one preferred embodiment utilizes the ICOS ABD having any of the variable heavy and light domain pairs of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In the Fab-Fc-IL15 x Fab-Fc-IL15Rα w/ ds format, one preferred embodiment utilizes the ICOS ABD having the variable heavy and light domain pair described herein, with either the IL-15 N4D/N65D variant or the IL-15 D30N/N65D variant or the IL-15 D30N/E64Q/N65D variant.

In the Fab-Fc-IL15 x Fab-Fc-IL15Rα w/ ds format, one preferred embodiment utilizes the skew variant pair S364K/E357Q:L368D/K370S.

In the Fab-Fc-IL15 x Fab-Fc-IL15Rα w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15 x Fab-Fc-IL15Rα w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/N65D variant or the IL-15 D30N/E64Q/N65D variant.

In the Fab-Fc-IL15 x Fab-Fc-IL15Rα w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

X. Fab-Fc-IL15-Fc x Fab-IL15Rα-Fc

This embodiment is shown in FIG. 25X, and comprises four monomers forming a tetramer. The first monomer comprises a VH-CH1-[optional domain linker]-IL-15 variant-[optional domain linker]-CH2-CH3, with the second optional domain linker sometimes being the hinge domain. The second monomer comprises a VH-CH1-[optional domain linker]-IL-15Rα(sushi) domain-[optional domain linker]-CH2-CH3, with the second optional domain linker sometimes being the hinge domain. The third (and fourth) monomers are light chains, VL-CL. This is generally referred to as “central-IL-15/Rα”.

The “Fab-Fc-IL15-Fc x Fab-IL15Rα-Fc” format (FIG. 25X) comprises a VH-CH1 recombinantly fused to the N-terminus of IL-15 which is then further fused to one side of a heterodimeric Fc and a VH-CH1 recombinantly fused to the N-terminus of IL-15Rα(sushi) which is then further fused to the other side of the heterodimeric Fc wherein each side of the heterodimeric Fc comprises complementary skew variants, while corresponding light chains are transfected separately so as to form Fabs with the VH-CH1s.

In the Fab-Fc-IL15-Fc x Fab-IL15Rα-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15-Fc x Fab-IL15Rα-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant, with the appropriate cysteine amino acid substitutions.

In the Fab-Fc-IL15-Fc x Fab-IL15Rα-Fc format, one preferred embodiment utilizes the skew variant pair S364K/E357Q:L368D/K370S.

In the Fab-Fc-IL15-Fc x Fab-IL15Rα-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15-Fc x Fab-IL15Rα-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/E64Q/N65D variant with appropriate cysteine substitutions.

In the Fab-Fc-IL15-Fc x Fab-IL15Rα-Fc format, one preferred embodiment utilizes the ICOS ABD the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

Y. Fab-Fc-IL15-Fc x Fab-IL15Rα-Fc w/ ds

This embodiment is shown in FIG. 25Y, and comprises four monomers forming a tetramer. The first monomer comprises a VH-CH1-[optional domain linker]-IL-15 variant-[optional domain linker]-CH2-CH3, with the second optional domain linker sometimes being the hinge domain, where the variant IL-15 domain has been engineered to contain a cysteine residue. The second monomer comprises a VH-CH1-[optional domain linker]-IL-15Rα(sushi) domain-[optional domain linker]-CH2-CH3, with the second optional domain linker sometimes being the hinge domain, where the variant IL-15Rα(sushi) has been engineered to contain a cysteine residue, such that the IL-15 complex is formed under physiological conditions. The third (and fourth) monomers are light chains, VL-CL. This is generally referred to as “Fab-Fc-IL15-Fc x Fab-IL15Rα-Fc w/ ds”.

In the Fab-Fc-IL15-Fc x Fab-IL15Rα-Fc w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15-Fc x Fab-IL15Rα-Fc w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant, with the appropriate cysteine amino acid substitutions.

In the Fab-Fc-IL15-Fc x Fab-IL15Rα-Fc w/ ds format, one preferred embodiment utilizes the skew variant pair S364K/E357Q:L368D/K370S.

In the Fab-Fc-IL15-Fc x Fab-IL15Rα-Fc w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-Fc-IL15-Fc x Fab-IL15Rα-Fc w/ ds format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/E64Q/N65D variant with appropriate cysteine substitutions.

In the Fab-Fc-IL15-Fc x Fab-IL15Rα-Fc w/ ds format, one preferred embodiment utilizes the ICOS ABD the variable heavy and light domain pair of [ICOS]_H0 and [ICOS]_L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

Z. Fab-IL15-Fc x Fab-IL15-Fc

This embodiment is shown in FIG. 25Z, and comprises four monomers forming a tetramer. The first and second monomer comprises a VH-CH1-[optional domain linker]-IL-15 variant-[optional domain linker]-CH2-CH3, with the second optional domain linker sometimes being the hinge domain. The third (and fourth) monomers are light chains, VL-CL. This is generally referred to as “Fab-IL15-Fc x Fab-IL15-Fc”.

In the Fab-Fc-IL15-Fc x Fab-IL15-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15-Fc x Fab-IL15-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, with either the IL-15 N4D/N65D variant or the IL-15 D30N/N65D variant or the IL-15 D30N/E64Q/N65D variant.

In the Fab-Fc-IL15-Fc x Fab-IL15-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-Fc-IL15-Fc x Fab-IL15-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S with either the IL-15 N4D/N65D variant or the IL-15 D30N/E64Q/N65D variant.

In the Fab-Fc-IL15-Fc x Fab-IL15-Fc format, one preferred embodiment utilizes the ICOS ABD the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the ablation variants E233P/L234V/L235A/G236_/S267K on both monomers, and optionally the 428L/434S variants on both sides.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

AA. Fab-scIL15Rα-IL15-Fc x Fab-Fc

This embodiment is shown in FIG. 25AA, and comprises four monomers forming a tetramer. The first monomer comprises a VH-CH1-[optional domain linker]-IL-15Rα(sushi) domain-domain linker-IL-15 variant-[optional domain linker]-CH2-CH3, with the second optional domain linker sometimes being the hinge domain. The second monomer comprises a VH-CH1-hinge-CH2-CH3. The third (and fourth) monomers are light chains, VL-CL. This is generally referred to as “Fab-scIL15Rα-IL15-Fc x Fab-Fc”, with the “sc” standing for “single chain”.

In the Fab-scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In the Fab-scIL15Rα-IL15-Fc x Fab-Fc format, one preferred embodiment utilizes the skew variant pair S364K/E357Q:L368D/K370S.

In the Fab-scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60. with either the IL-15 N4D/N65D variant or the IL-15 D30N/N65D variant or the IL-15 D30N/E64Q/N65D variant.

In the Fab-scIL15Rα-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

BB. Fab-scIL15-IL15Rα-Fc x Fab-Fc

This embodiment is shown in FIG. 25BB, and comprises four monomers forming a tetramer. The first monomer comprises a VH-CH1-[optional domain linker]-IL-15 variant-domain linker-IL-15Rα(sushi) domain-[optional domain linker]-CH2-CH3, with the second optional domain linker sometimes being the hinge domain. The second monomer comprises a VH-CH1-hinge-CH2-CH3. The third (and fourth) monomers are light chains, VL-CL. This is generally referred to as “Fab-scIL15-IL15Rα-Fc x Fab-Fc”, with the “sc” standing for “single chain”.

In the Fab-scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In the Fab-scIL15-IL15Rα-Fc x Fab-Fc format, one preferred embodiment utilizes the skew variant pair S364K/E357Q:L368D/K370S.

In the Fab-scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60. with either the IL-15 N4D/N65D variant or the IL-15 D30N/N65D variant or the IL-15 D30N/E64Q/N65D variant.

In the Fab-scIL15-IL15Rα-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

CC. Fab-IL15-Fc x Fab-Fc

This embodiment is shown in FIG. 25CC, and comprises four monomers forming a tetramer. The first monomer comprises a VH-CH1-[optional domain linker]-IL-15 variant-[optional domain linker]-CH2-CH3, with the second optional domain linker sometimes being the hinge domain. The second monomer comprises a VH-CH1-hinge-CH2-CH3. The third (and fourth) monomers are light chains, VL-CL. This is generally referred to as “Fab-IL15-Fc x Fab-Fc”.

In the Fab-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60.

In the Fab-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In the Fab-IL15-Fc x Fab-Fc format, one preferred embodiment utilizes the skew variant pair S364K/E357Q:L368D/K370S.

In the Fab-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60 and the skew variant pair S364K/E357Q:L368D/K370S.

In the Fab-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60. with either the IL-15 N4D/N65D variant or the IL-15 D30N/N65D variant or the IL-15 D30N/E64Q/N65D variant.

In the Fab-IL15-Fc x Fab-Fc format, one preferred embodiment utilizes the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; or STIM003[ICOS] vh and vl from FIG. 60, the skew variants S364K/E357Q (on a first Fc-containing monomer) and L368D/K370S (on the second corresponding Fc-containing monomer), the pI variants Q295E/N384D/Q418E/N421D (on either the first or the second Fc-containing monomer; and further comprising N208D if said Fc-containing monomer comprises CH1), the ablation variants E233P/L234V/L235A/G236_/S267K on both Fc-containing monomers, and optionally the 428L/434S variants on both Fc-containing monomers.

Additional useful human ICOS antigen binding domains for use in this ICOS-targeted x IL-15/IL-15Rα Fc fusion protein format include, but are not limited to, those listed in FIGS. 24, 60, and 62-64 (e.g., SEQ ID NOs:140-143, 179-242, 315-584) and the sequence listing (e.g., SEQ ID NOs:585-802).

### IV. Particularly Useful Embodiments of the Invention

The present invention provides a ICOS-targeted IL-15/IL-15Rα heterodimeric fusion protein comprising at least two monomers, one of which contains an ICOS ABD and the other that contains an IL-15/RA complex, joined using heterodimeric Fc domains.

In some embodiments, the first and the second Fc domains have a set of amino acid substitutions selected from the group consisting of S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L and K370S:S364K/E357Q, and T366S/L368A/Y407V:T366W (optionally including a bridging disulfide, T366S/L368A/Y407V/Y349C:T366W/S354C or T366S/L368A/Y407V/5354C:T366W:Y349C, according to EU numbering.

In some instances, the first and/or the second Fc domains have an additional set of amino acid substitutions comprising Q295E/N384D/Q418E/N421D, according to EU numbering. In some cases, the first and/or the second Fc domains have an additional set of amino acid substitutions consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to EU numbering.

In some embodiments, the first and the second Fc domains have an amino acid substitution comprising M428L/N434S or M428L/N434A.

In some embodiments, the IL-15 protein has a polypeptide sequence selected from the group consisting of SEQ ID NO:1 (full-length human IL-15) and SEQ ID NO:2 (truncated human IL-15), and the IL-15Rα protein has a polypeptide sequence selected from the group consisting of SEQ ID NO:3 (full-length human IL-15Rα) and SEQ ID NO:4 (sushi domain of human IL-15Rα).

In some embodiments, the IL-15 protein and the IL-15Rα protein can have a set of amino acid substitutions selected from the group consisting of E87C:D96/P97/C98; E87C:D96/C97/A98; V49C:540C; L52C:540C; E89C:K34C; Q48C:G38C; E53C:L42C; C42S:A37C; and L45C:A37C, respectively.

In some embodiments, the IL-15 protein variant has amino acid substitutions selected from N4D/N65D, D30N/N65D, or D30N/E64Q/N65D.

In some embodiments, provided herein is a heterodimeric fusion protein of a scIL-15/Rα x Fab format comprising: (a) a first monomer comprising, from N- to C-terminal: i) an IL-15 sushi domain; ii) a first domain linker; iii) a variant IL-15 domain; iv) a second domain linker; v) a first variant Fc domain comprising CH2-CH3; and (b) a second monomer comprising a heavy chain comprising VH1-CH1-hinge-CH2-CH3, wherein the CH2-CH3 is a second variant Fc domain; and c) a light chain comprising VL-CL; wherein the VH1 and VL form an antigen binding domain that binds human ICOS.

In some embodiments, the ICOS antigen binding domain comprises an anti-ICOS scFv or an anti-ICOS Fab.

In some embodiments, the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

In some embodiments, the ICOS ABD having the variable heavy and light domain pair of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60, with either the IL-15 N4D/N65D variant or the IL-15 D30N/N65D variant or the IL-15 D30N/E64Q/N65D variant.

In preferred embodiments, the ICOS-targeted x IL-15/RA Fc fusion protein of the present disclosure has a scIL-15/Rα x Fab format. In some cases, the IL-15Rα(sushi) domain is fused to the N-terminus of the IL-15 variant by a linker and the C-terminus of the IL-15 variant is fused to the N-terminus of one side of a heterodimeric Fc-region. The heterodimeric Fc-region includes a variable heavy chain fused to the other side of the heterodimeric Fc. The corresponding variable light chain forms a Fab with the variable heavy chain.

In some embodiments, the ICOS-targeted IL-15/RA-Fc fusion protein is XENP29975, XENP29978, XENP30810, XENP30811, XENP30812, or XENP30813.

In some embodiments, the ICOS-targeted IL-15/RA-Fc fusion protein is depicted in FIGS. 26A-26D.

In some embodiments, the ICOS-targeted IL-15/RA-Fc fusion protein is depicted in FIGS. 61A-61P.

### V. Nucleic Acids of the Invention

The invention further provides nucleic acid compositions encoding the targeted heterodimeric fusion proteins of the invention (or, in the case of a monomer Fc domain protein, nucleic acids encoding those as well).

As will be appreciated by those in the art, the nucleic acid compositions will depend on the format of the targeted heterodimeric fusion protein. Thus, for example, when the format requires three amino acid sequences, three nucleic acid sequences can be incorporated into one or more expression vectors for expression. In any embodiments, each of the three coding nucleic acid sequences are incorporated into different expression vectors. Similarly, some formats only two nucleic acids are needed; again, they can be put into one or two expression vectors, or four or 5. As noted herein, some constructs have two copies of a light chain, for example.

As is known in the art, the nucleic acids encoding the components of the invention can be incorporated into expression vectors as is known in the art, and depending on the host cells used to produce the targeted heterodimeric fusion proteins of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer, as applicable depending on the format, are each contained within a single expression vector, generally under different or the same promoter controls. In embodiments of particular use in the present invention, each of these two or three nucleic acids are contained on a different expression vector.

The targeted heterodimeric fusion proteins of the invention are made by culturing host cells comprising the expression vector(s) as is well known in the art. Once produced, traditional fusion protein or antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the heterodimer (e.g., anionic exchange columns, cationic exchange columns). These substitutions also aid in the determination and monitoring of any contaminating homodimers post-purification (e.g., IEF gels, cIEF, and analytical IEX columns).

### VI. Biological and Biochemical Functionality of Targeted ICOS Antibody x IL-15/IL-15Rα Heterodimeric Immunomodulatory Fusion Proteins

Generally, the targeted heterodimeric fusion proteins of the invention are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status along with “old fashioned” measurements such as tumor burden, size, invasiveness, LN involvement, metastasis, etc. can be done. Thus, any or all of the following can be evaluated: the inhibitory effects of the heterodimeric fusion proteins on CD4⁺ T cell activation or proliferation, CD8⁺ T (CTL) cell activation or proliferation, CD8⁺ T cell-mediated cytotoxic activity and/or CTL mediated cell depletion, NK cell activity and NK mediated cell depletion, the potentiating effects of the heterodimeric fusion protein on Treg cell differentiation and proliferation and Treg- or myeloid derived suppressor cell (MDSC)-mediated immunosuppression or immune tolerance, and/or the effects of heterodimeric fusion protein on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and ³H-thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, ⁵¹Cr or 35S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

A. Assays to Measure Efficacy

In some embodiments, T cell activation is assessed using a Mixed Lymphocyte Reaction (MLR) assay as is known in the art. An increase in activity indicates immunostimulatory activity. Appropriate increases in activity are outlined below.

In one embodiment, the signaling pathway assay measures increases or decreases in immune response as measured for an example by phosphorylation or de-phosphorylation of different factors, or by measuring other post translational modifications. An increase in activity indicates immunostimulatory activity. Appropriate increases in activity are outlined below.

In one embodiment, the signaling pathway assay measures increases or decreases in activation of αβ and/or γδ T cells as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in cytotoxic T cell activity as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in NK and/or NKT cell activity as measured for an example by direct killing of target cells like for an example cancer cells or by cytokine secretion or by changes in expression of activation markers like for an example CD107a, etc. An increase in activity indicates immunostimulatory activity. Appropriate increases in activity are outlined below.

In one embodiment, the signaling pathway assay measures increases or decreases in αβ and/or γδ T-cell suppression, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in pro-inflammatory cytokine secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined below.

In one embodiment, the signaling pathway assay measures increases or decreases in Th1 response as measured for an example by cytokine secretion or by changes in expression of activation markers. An increase in activity indicates immunostimulatory activity. Appropriate increases in activity are outlined below.

In one embodiment, the signaling pathway assay measures increases or decreases in Th2 response as measured for an example by cytokine secretion or by changes in expression of activation markers. An increase in activity indicates immunostimulatory activity. Appropriate increases in activity are outlined below.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined below.

In one embodiment, the signaling pathway assay measures increases or decreases in M2 macrophage pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined below.

In one embodiment, the signaling pathway assay measures increases or decreases in N2 neutrophils pro-tumorigenic activity, as measured for an example by cytokine secretion or by changes in expression of activation markers. A decrease in response indicates immunostimulatory activity. Appropriate decreases are the same as for increases, outlined below.

In one embodiment, the signaling pathway assay measures increases or decreases in inhibition of T cell activation, as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in inhibition of CTL activation as measured for an example by direct killing of target cells like for an example cancer cells or by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in αβ and/or γδ T cell exhaustion as measured for an example by changes in expression of activation markers. A decrease in response indicates immunostimulatory activity. Appropriate decreases are the same as for increases, outlined below.

In one embodiment, the signaling pathway assay measures increases or decreases αβ and/or γδ T cell response as measured for an example by cytokine secretion or by proliferation or by changes in expression of activation markers like for an example CD137, CD107a, PD1, etc. An increase in activity indicates immunostimulatory activity. Appropriate increases in activity are outlined below.

In one embodiment, the signaling pathway assay measures increases or decreases in stimulation of antigen-specific memory responses as measured for an example by cytokine secretion or by proliferation or by changes in expression of activation markers like for an example CD45RA, CCR7 etc. An increase in activity indicates immunostimulatory activity. Appropriate increases in activity are outlined below.

In one embodiment, the signaling pathway assay measures increases or decreases in apoptosis or lysis of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases in stimulation of cytotoxic or cytostatic effect on cancer cells. as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases direct killing of cancer cells as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, the signaling pathway assay measures increases or decreases Th17 activity as measured for an example by cytokine secretion or by proliferation or by changes in expression of activation markers. An increase in activity indicates immunostimulatory activity. Appropriate increases in activity are outlined below.

In one embodiment, the signaling pathway assay measures increases or decreases in induction of complement dependent cytotoxicity and/or antibody dependent cell-mediated cytotoxicity, as measured for an example by cytotoxicity assays such as for an example MTT, Cr release, Calcine AM, or by flow cytometry based assays like for an example CFSE dilution or propidium iodide staining etc. An increase in activity indicates immunostimulatory activity. Appropriate increases in activity are outlined below.

In one embodiment, T cell activation is measured for an example by direct killing of target cells like for an example cancer cells or by cytokine secretion or by proliferation or by changes in expression of activation markers like for an example CD137, CD107a, PD1, etc. For T-cells, increases in proliferation, cell surface markers of activation (e.g. CD25, CD69, CD137, PD1), cytotoxicity (ability to kill target cells), and cytokine production (e.g. IL-2, IL-4, IL-6, IFNγ, TNF-a, IL-10, IL-17A) would be indicative of immune modulation that would be consistent with enhanced killing of cancer cells.

In one embodiment, NK cell activation is measured for example by direct killing of target cells like for an example cancer cells or by cytokine secretion or by changes in expression of activation markers like for an example CD107a, etc. For NK cells, increases in proliferation, cytotoxicity (ability to kill target cells and increases CD107a, granzyme, and perforin expression), cytokine production (e.g. IFNγ and TNF), and cell surface receptor expression (e.g. CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of 10%, 20%, 30%, 40%, 50%, 60%, 70%, 80%, 90%, 95% or 98 to 99% percent over the signal in either a reference sample or in control samples, for example test samples that do not contain a heterodimeric fusion protein of the invention. Similarly, increases of at least one-, two-, three-, four- or five-fold as compared to reference or control samples show efficacy.

### VII. Treatments

Once made, the compositions of the invention find use in a number of oncology applications, by treating cancer, generally by promoting T cell activation (e.g., T cells are no longer suppressed) with the binding of the heterodimeric Fc fusion proteins of the invention.

Accordingly, the targeted heterodimeric compositions of the invention find use in the treatment of these cancers.

A. Targeted Heterodimeric Fusion Protein Compositions for In Vivo Administration

Formulations of the antibodies used in accordance with the present invention are prepared for storage by mixing an antibody having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions. Acceptable carriers, buffers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

B. Combination Therapies

In some embodiments, the heterodimeric fusion proteins of the invention can be used in combination therapies with antibodies that bind to different checkpoint proteins, e.g. not ICOS antibodies. In this way, the antigen binding domains of the additional antibody do not compete for binding with the targeted heterodimeric fusion protein. In this way, a sort of “triple combination” therapy is achieved, as three receptors are engaged (two from the targeted heterodimeric fusion protein and one from the additional antibody). As discussed herein, the heterodimeric fusion protein can have different valencies and specificities as outlined herein.

Surprisingly, as shown herein, these combinations can result in synergistic effects when co-administered. In this context, “co-administration” means that the two moieties can be administered simultaneously or sequentially. That is, in some cases, the drugs may be administered simultaneously, although generally this is through the use of two separate IV infusions; that is, the drugs are generally not combined into a single dosage unit. Alternatively, co-administration includes the sequential administration of the two separate drugs, either in a single day or separate days (including separate days over time).

1. Anti-PD-1 Antibodies for Use in Co-Administration Therapies

As is known in the art, there are two currently approved anti-PD-1 antibodies and many more in clinical testing. Thus, suitable anti-PD-1 antibodies for use in combination therapies as outlined herein include, but are not limited to, the two currently FDA approved antibodies, pembrolizumab and nivolizumab, as well as those in clinical testing currently, including, but not limited to, tislelizumab, Sym021, REGN2810 (developed by Rengeneron), JNJ-63723283 (developed by J and J), SHR-1210, pidilizumab, AMP-224, MEDIo680, PDR001 and CT-001, as well as others outlined in Liu et al., J. Hemat. & Oncol. (2017), 10:136, the antibodies therein expressly incorporated by reference. As above, anti-PD-1 antibodies are used in combination when the targeted IL-15/IL-15Rα-Fc fusion protein of the invention do not have an antigen binding domain that binds PD-1.

2. Anti-PD-L1 Antibodies for Use in Co-Administration Therapies

In some embodiments, anti-PD-L1 antibodies are used in combination. As is known in the art, there are three currently approved anti-PD-L1 antibodies and many more in clinical testing. Thus, suitable anti-PD-L1 antibodies for use in combination therapies as outlined herein include, but are not limited to, the three currently FDA approved antibodies, atezolizumab, avelumab, durvalumab, as well as those in clinical testing currently, including, but not limited to, LY33000054 and CS1001, as well as others outlined in Liu et al., J. Hemat. & Oncol. (2017), 10:136, the antibodies therein expressly incorporated by reference. As above, anti-PD-L1 antibodies are used in combination when the targeted IL-15/IL-15Rα-Fc fusion protein of the invention do not have an antigen binding domain that binds PD-L1.

3. Anti-TIM-3 Antibodies for Use in Co-Administration Therapies

In some embodiments, anti-TIM-3 antibodies can be used in combination with the targeted IL-15/IL-15Rα-Fc fusion proteins of the invention. There are several TIM-3 antibodies in clinical development, including MBG453 and TSR-022. As above, anti-TIM-3 antibodies are used in combination when the targeted IL-15/IL-15Rα-Fc fusion protein of the invention do not have an antigen binding domain that binds TIM-3.

4. Anti-LAG-3 Antibodies for Use in Co-Administration Therapies

In some embodiments, anti-LAG-3 antibodies can be used in combination with the targeted IL-15/IL-15Rα-Fc fusion proteins of the invention. There are several LAG-3 antibodies in clinical development including BMS-986016, LAG525 and REGN3767. As above, anti-LAG-3 antibodies are used in combination when the targeted IL-15/IL-15Rα-Fc fusion protein of the invention do not have an antigen binding domain that binds LAG-3.

5. Anti-CTLA-4 Antibodies for Use in Co-Administration Therapies

In some embodiments, anti-CTLA-4 antibodies can be used in combination with the targeted IL-15/IL-15Rα-Fc fusion protein of the invention. Ipilimumab has been approved, and there are several more in development, including CP-675,206 and AGEN-1884. As above, anti-CTLA-4 antibodies are used in combination when the targeted IL-15/IL-15Rα-Fc fusion protein of the present invention do not have an antigen binding domain that binds CTLA-4.

6. Anti-TIGIT Antibodies for Use in Co-Administration Therapies

In some embodiments, anti-TIGIT antibodies can be used in combination with the targeted IL-15/IL-15Rα-Fc fusion proteins of the invention.

C. Administrative Modalities

The targeted heterodimeric fusion proteins and chemotherapeutic agents of the invention are administered to a subject, in accord with known methods, such as intravenous administration as a bolus or by continuous infusion over a period of time.

D. Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the targeted heterodimeric fusion protein used in the present invention depend on the disease or condition to be treated and may be determined by the persons skilled in the art.

An exemplary, non-limiting range for a therapeutically effective amount of a targeted heterodimeric fusion protein used in the present invention is about 0.1-100 mg/kg.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### VIII. Examples

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Patent Publications 2015/0307629, 2014/0288275 and WO2014/145806, all of which are expressly incorporated by reference in their entirety and particularly for the techniques outlined therein. Examples 1 and 2 from U.S. Ser. No. 62/416,087, filed on Nov. 1, 2016 are expressly incorporated by reference in their entirety, including the corresponding figures. Additionally, U.S. Ser. Nos. 62/408,655, 62/443,465, 62/477,926, 15/785,401, 62/416,087 and 15/785,393 are expressly incorporated by reference in their entirety, and specifically for all the sequences, Figures and Legends therein.

### A. Example 1: IL-15/Rα-Fc

1. 1A: Engineering IL-15/Rα-Fc Fusion Proteins

In order to address the short half-life of IL-15/IL-15Rα heterodimers, we generated the IL-15/IL-15Rα(sushi) complex as an Fc fusion (herein, collectively referred to as IL-15/Rα-Fc fusion proteins) with the goal of facilitating production and promoting FcRn-mediated recycling of the complex and prolonging half-life.

Plasmids coding for IL-15 or IL-15Rα sushi domain were constructed by standard gene synthesis, followed by subcloning into a pTT5 expression vector containing Fc fusion partners (e.g., constant regions as depicted in FIG. 11). Cartoon schematics of illustrative IL-15/Rα-Fc fusion protein formats are depicted in FIGS. 14A-G.

An illustrative protein of the IL-15/Rα-heteroFc format (FIG. 14A) is XENP20818, sequences for which are depicted in FIG. 15, with sequences for additional proteins of this format. An illustrative proteins of the scIL-15/Rα-Fc format (FIG. 14B) is XENP21478, sequences for which are depicted in FIG. 16. An illustrative proteins of the ncIL-15/Rα-Fc format (FIG. 14C) is XENP21479, sequences for which are depicted in FIG. 17.

Proteins were produced by transient transfection in HEK293E cells and were purified by a two-step purification process comprising protein A chromatography and ion exchange chromatography.

Illustrative IL-15/Rα-Fc fusion proteins in the scIL-15/Rα-Fc format (XENP21478) and in the ncIL-15/Rα-Fc format (XENP21479) were tested in a cell proliferation assay. Human PBMCs were treated with the test articles at the indicated concentrations. 4 days after treatment, the PBMCs were stained with anti-CD8-FITC (RPA-T8), anti-CD4-PerCP/Cy5.5 (OKT4), anti-CD27-PE (M-T271), anti-CD56-BV421 (5.1H11), anti-CD16-BV421 (3G8), and anti-CD45RA-BV605 (Hi100) to gate for the following cell types: CD4+ T cells, CD8+ T cells, and NK cells (CD56+/CD16+). Ki67 is a protein strictly associated with cell proliferation, and staining for intracellular Ki67 was performed using anti-Ki67-APC (Ki-67) and Foxp3/Transcription Factor Staining Buffer Set (Thermo Fisher Scientific, Waltham, Mass.). The percentage of Ki67 on the above cell types was measured using FACS (depicted in FIGS. 18A-C). The data show that the illustrative IL-15/Rα-Fc fusion proteins induced strong proliferation of CD8+ T cells and NK cells.

2. 1B:IL-15/Rα-Fc Fusion Proteins Engineered for Lower Potency

In order to further improve PK and prolong half-life, we reasoned that decreasing the potency of IL-15/Rα-Fc fusions would decrease the antigen sink, and thus, increase circulating half-life. By examining the crystal structure of the IL-15:IL-2Rβ and IL-15:common gamma chain interfaces, as well as by modeling using MOE software, we predicted residues at these interfaces that may be substituted in order to reduce potency. FIG. 19 depicts a structural model of the IL-15:receptor complexes showing locations of the predicted residues where we engineered isosteric substitutions (in order to reduce the risk of immunogenicity). Sequences for illustrative IL-15 variants engineered with the aim to reduce potency are depicted in FIG. 20.

Plasmids coding for IL-15 or IL-15Rα(sushi) were constructed by standard gene synthesis, followed by subcloning into a pTT5 expression vector containing Fc fusion partners (e.g., constant regions as depicted in FIG. 11). Substitutions identified as described above were incorporated by standard mutagenesis techniques. Sequences for illustrative IL-15/Rα-Fc fusion proteins of the “scIL-15/Rα-Fc” format engineered for reduced potency are depicted in FIG. 21 Proteins were produced and purified as generally described in Example 1A.

a. 1B(a): In Vitro Activity of scIL-15/Rα-Fc Fusion Proteins Comprising IL-15 Variants Engineered for Decreased Potency

Illustrative scIL-15/Rα-Fc fusion proteins comprising IL-15 variants were tested in cell proliferation assays. Human PBMCs were incubated with the indicated test articles at the indicated concentrations for 3 days. Following incubation, the PBMCs were stained with anti-CD3-PE (OKT3), anti-CD4-FITC (RPA-T4), anti-CD8-eF660 (SIDI8BEE), anti-CD16-BV421 (3G8), anti-CD45RA-APC/Fire750 (HI100), anti-CD56-BV605 (5.1H11), and anti-Ki67-PE/Cy7 (Ki-67) and analyzed by flow cytometry. FIG. 22 depicts the percentage of various lymphocyte populations expressing Ki67 indicative of proliferation.

The data show that several of the illustrative scIL-15/Rα-Fc fusions comprising IL-15 variants engineered with the aim to reduce potency did demonstrate reduced potency relative to scIL-15/Rα-Fc fusions comprising WT IL-15. Notably, the data show that scIL-15/Rα-Fc fusions comprising IL-15(D30N/E64Q/N65D) variant had drastically reduced activity in proliferation of various lymphocyte populations in the context of scIL-15/Rα-Fc fusions, in comparison to scIL-15/Rα-Fc fusions comprising IL-15(N4D/N65D) or IL-15(D30N/N65D) variants. On the other hand, scIL-15/Rα-Fc fusion comprising IL-15(D30N) variant had little to no reduction in potency relative to scIL-15/Rα-Fc fusion comprising WT IL-15.

### B. Example 2: ICOS-Targeted IL-15/Rα-Fc Fusions

Here, we describe the generation and characterization of IL-15/Rα-Fc fusions targeted to ICOS, collectively referred to herein as ICOS-targeted IL-15/Rα-Fc fusions.

1. 2A: Engineering ICOS-Targeted IL-15/Rα-Fc Fusions

Plasmids coding for IL-15, IL-15Rα sushi domain, or the anti-ICOS variable regions were constructed by standard gene synthesis, followed by subcloning into a pTT5 expression vector containing Fc fusion partners (e.g., constant regions as depicted in FIG. 12). Cartoon schematics of illustrative ICOS-targeted IL-15/Rα-Fc fusions are depicted in FIG. 25.

A particular illustrative format, the “scIL-15/Rα x Fab” format (FIG. 25C), comprises IL-15Rα(sushi) fused to IL-15 by a variable length linker (termed “scIL-15/Rα”) which is then fused to the N-terminus of a heterodimeric Fc-region, with a variable heavy chain (VH) fused to the other side of the heterodimeric Fc, while a corresponding light chain is transfected separately so as to form a Fab with the VH.

We generated ICOS-targeted IL-15/Rα-Fc fusions in this format with illustrative anti-ICOS variable regions as depicted in FIG. 24, and the IL-15(N4D/N65D) variant. Sequences for XENP29975, an illustrative ICOS-targeted IL-15/Rα-Fc fusion protein with IL-15(N4D/N65D) variant as such (and Xtend analog XENP30811), are depicted in FIG. 26, with additional sequences depicted in FIG. 61 (based on additional anti-ICOS variable regions depicted in FIG. 60). We also generated a control RSV-targeted IL-15/Rα-Fc fusion protein XENP26007 with IL-15(N4D/N65D) variant, sequences for which are depicted in FIG. 27.

Proteins were produced by transient transfection in HEK293E cells and were purified by a two-step purification process comprising protein A chromatography and ion exchange chromatography.

2. 2B: ICOS-Targeted IL-15/Rα-Fc Fusions are Active In Vitro

Human PBMCs (from 2 donors in two separate experiments) were stimulated for 48 hours with 500 ng/ml plate-bound anti-CD3 (OKT3) and then labeled with CFSE and incubated with the following test articles for 4 days at 37° C.: XENP29975 (ICOS-targeted IL-15/Rα-Fc fusion having N4D/N65D IL-15 variant); XENP24306 (control untargeted IL-15/Rα-Fc fusion having D30N/E64Q/N65D IL-15 variant); and XENP26007 (control RSV-targeted IL-15/Rα-Fc fusion having N4D/N65D IL-15 variant). Cells were stained with the following antibodies: anti-CD8-PerCP-By5.5 (SK1), anti-CD3-PE-Cy7 (OKT3), anti-PD-1-Alexa647 (XENP164352, sequences depicted in FIG. X, stained with Alexa Fluor™ 647 Antibody Labeling Kit), anti-CD45RO-APC-Fire750 (UCHL1), anti-HLA-DR-Alexa700 (L243), anti-CD107a-BV421 (H4A3), anti-CD16-BV605 (3G6), anti-CD56-BV605 (HCD56), anti-CD25-BV711 (M-A251), anti-CD45RA-BV785 (M-A251), anti-CD4-BUV395 (SK3), and Zombie Aqua (BV510), and analyzed by flow cytometry for various cell populations.

We investigated the proliferation of various T cell populations based on CFSE dilution (Zombie Aqua to exclude dead cells), data for which are depicted in FIGS. 28-36. While there are some variability between donors, the data generally suggests that ICOS-targeted IL-15/Rα-Fc fusions are much more potent in inducing proliferation of both CD8⁺ and CD4⁺ T cells in comparison to untargeted IL-15/Rα-Fc fusion (as well as control RSV-targeted IL-15/Rα-Fc fusion). Additionally, as shown in FIG. 37-38, ICOS-targeted IL-15/Rα-Fc fusions are much less potent in inducing proliferation of NK cells.

We also investigate the activation of various T cell populations based on expression of CD25 (a late stage T cell activation marker), data for which are depicted in FIGS. 39-47. Again, while there are some variability between the donors, the data generally suggest that ICOS-targeted IL-15/Rα-Fc fusions are more potent in inducing activation of CD8⁺CD45RA⁻, CD8⁺CD45RA⁺, and CD4⁺CD45RA⁻ T cells in comparison to untargeted IL-15/Rα-Fc fusion (as well as control RSV-targeted IL-15/Rα-Fc fusion).

Further, we investigated the expression of HLA-DR (another activation marker) on various T cell populations, data for which are depicted in FIG. 48-54Y.

### C. Example 3: Generation of ICOS-Targeted IL-15/Rα-Fc Fusions Having Alternative IL-15 Potency Variants

1. 3A: IL-15-Fc Fusions Comprising IL-15(N4D/N65D) Variant Demonstrate Reduced Pharmacokinetics

In a study investigating the pharmacokinetics of IL-15-Fc potency variants with Xtend, cynomolgus monkeys were administered a first single intravenous (i.v.) dose of XENP22853 (WT IL-15/Rα-heteroFc with Xtend), XENP24306 (IL-15(D30N/E64Q/N65D)/Rα-heteroFc with Xtend), XENP24113 (IL-15(N4D/N65D)/Rα-heteroFc with Xtend), and XENP24294 (scIL-15(N4D/N65D)/Rα-Fc with Xtend) at varying concentrations.

FIG. 59 depicts the serum concentration of the test articles over time following the first dose. As expected, incorporating potency variants in addition to Xtend substitution (as in XENP24306 and XENP24113) greatly improves the pharmacokinetics of IL-15-Fc fusions (in comparison to XENP22583). Unexpectedly, however, IL-15/Rα-heteroFc fusion XENP24113 and scIL-15/Rα-Fc fusion XENP24294 (which have the same IL-15(N4D/N65D) potency variant) demonstrated reduced pharmacokinetics in comparison to XENP24306. This suggests that the reduced pharmacokinetics was due to the particular IL-15 potency variant rather than the format of the IL-15-Fc fusion. While a decrease in pharmacokinetics for XENP24113 and XENP24294 was expected on the basis of previous findings which demonstrated that the IL-15-Fc fusions having IL-15(N4D/N65D) variant had greater in vitro potency than IL-15-Fc fusions having the IL-15(D30N/E64Q/N65D) variant, the decrease in pharmacokinetics was unexpectedly disproportionate to the increase in potency. Accordingly, we sought to identify alternative IL-15 potency variants for use in the LAG-3-targeted IL-15-Fc fusions of the invention.

2. 3B: ICOS-Targeted IL-15-Fc Fusions Comprising IL-15(D30N/N65D)

We noted that IL-15(N4D/N65D) has both its substitutions at the IL-15 interface responsible for binding to CD122, while IL-15(D30N/E64Q/N65D) has two substitutions (E64Q and N65D) at IL-15:CD122 interface; and one substitution (D30N) at the IL-15 interface responsible for binding to CD132. Accordingly, we reasoned that the modification at the IL-15:CD132 interface may contribute to the superior pharmacokinetics observed for XENP24306. In view of the above, we produced illustrative ICOS-targeted IL-15-Fc fusion XENP29978 comprising the IL-15(D30N/N65D) variant (and Xtend analog XENP30812), sequences for which are depicted in FIG. 26, with additional sequences depicted in FIG. 61. We also generated a control RSV-targeted IL-15/Rα-Fc fusion protein XENP29481 with IL-15(D30N/N65D) variant, sequences for which are depicted in FIG. 27.

3. 3C: ICOS-Targeted IL-15-Fc Fusions Comprising IL-15(D30N/E64Q/N65D)

Although the ICOS-targeted IL-15/Rα-Fc fusions were designed with targeting to the tumor environment via the ICOS-targeting arm in mind, the cytokine moiety is still capable of signaling before reaching the tumor site and may contribute to systemic toxicity. Accordingly, we sought to further reduce the IL-15 potency by constructing ICOS-targeted IL-15/Rα-Fc fusions with IL-15(D30N/E64Q/N65D) variant, which as described in Example 1B(a) has drastically reduced activity. Sequences for illustrative ICOS-targeted IL-15/Rα-Fc fusions comprising IL-15(D30N/E64Q/N65D) variant are depicted in FIG. 26 as XENP30810 (and Xtend analog as XENP30813), with additional sequences depicted in FIG. 61. Additionally, we constructed XENP30432, a RSV-targeted IL-15/Rα-Fc fusion comprising IL-15(D30N/E64Q/N65D) variant (sequences for which are depicted in FIG. 27) to act as a surrogate for investigating the behavior of ICOS-targeted IL-15/Rα-Fc fusions comprising IL-15(D30N/E64Q/N65D) variant outside of the tumor environment.

1. (canceled)

2. A heterodimeric protein comprising:
a) a first monomer comprising, from N- to C-terminal:
i) an IL-15Rα sushi domain;
ii) a first domain linker;
iii) a variant IL-15 domain;
iv) a second domain linker;
v) a first variant Fc domain comprising CH2-CH3; and
b) a second monomer comprising a heavy chain comprising VH1-CH1-hinge-CH2-CH3, wherein said CH2-CH3 is a second variant Fc domain; and
c) a light chain comprising VL-CL;
wherein said VH1 and VL form an antigen binding domain that binds human ICOS.

3.-30. (canceled)

31. The heterodimeric protein according to claim 2 wherein said VH and VL are selected from the pairs selected from the group consisting of [ICOS]_H0 and [ICOS]L0 from FIG. 24; [ICOS]_H0.66 and [ICOS]_L0 from FIG. 24; Jmab-136[ICOS]vh and vl from FIG. 60; ICOS 314.8[ICOS]vh and vl from FIG. 60; H2L5[ICOS] vh and vl from FIG. 60; 37A10S713[ICOS] vh and vl from FIG. 60; C398.4A[ICOS] vh and vl from FIG. 60; ICOS.33[ICOS] vh and vl from FIG. 60; STIM001[ICOS] vh and vl from FIG. 60; and STIM003[ICOS] vh and vl from FIG. 60.

32. The heterodimeric protein according to claim 2 wherein the first and the second Fc domains have a set of amino acid substitutions selected from the group consisting of S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W; T366S/L368A/Y407V/Y349C:T366W/S354C; and T366S/L368A/Y407V/S354C:T366W/Y349C, according to EU numbering.

33. The heterodimeric protein according to claim 2 wherein the first and the second Fc domains have S364K/E357Q:L368D/K370S.

34. The heterodimeric protein according to claim 2 wherein said variant Fc domains each comprise M428L/N434S.

35. The heterodimeric protein according to claim 2 wherein said variant Fc domains each comprise E233P/L234V/L235A/G236del/S267K.

36. The heterodimeric protein according to claim 2 wherein said variant IL-15 domain comprises an amino acid substitution(s) selected from the group consisting of N1D, N4D, D8N, D30N, V49R, D61N, E64Q, N65D, N72D, Q108E, N4D/N65D, D30N/N65D, D30N/E64Q/N65D, N1G/D30N/E46G/V49R/E64Q, N1A/D30N/E46G/V49R, and D22N/Y26F/E46Q/E53Q/E89Q/E93Q.

37. A heterodimeric protein according to claim 2 selected from the group consisting of XENP29975, XENP29978, XENP30810, XENP30811, XENP30812 and XENP30813.

38.-40. (canceled)
